(19) United States
(12) Patent Application Publication
(10) Pub. No.: US 2025/0290139 A1
(43) Pub. Date: Sep. 18, 2025

(54) DIAGNOSIS AND PROGNOSIS OF RICHTER'S SYNDROME

(71) Applicants: The Broad Institute, Inc., Cambridge, MA (US); Dana-Farber Cancer Institute, Boston, MA (US); The General Hospital Corporation, Boston, MA (US); Trustees of Boston University, Boston, MA (US)

(72) Inventors: Erin Michelle Parry, Boston, MA (US); Ignaty Leshchiner, Cambridge, MA (US); Romain Guieze, Boston, MA (US); Catherine Wu, Boston, MA (US); Gad Getz, Boston, MA (US)

(73) Assignees: The Broad Institute, Inc., Cambridge, MA (US); Dana-Farber Cancer Institute, Boston, MA (US); The General Hospital Corporation, Boston, MA (US); Trustees of Boston University, Boston, MA (US)

(21) Appl. No.: 18/605,472

(22) Filed: Mar. 14, 2024

Related U.S. Application Data
(63) Continuation of application No. PCT/US2022/043647, filed on Sep. 15, 2022.
(60) Provisional application No. 63/244,625, filed on Sep. 15, 2021.
(60) Provisional application No. 63/291,213, filed on Dec. 17, 2021.

(51) Int. Cl. C12Q 1/6883; G16B 20/20; G16H 50/20

(52) U.S. Cl. CPC C12Q 1/6883; G16B 20/20; G16H 50/20; C12Q 2600/112; C12Q 2600/118; C12Q 2600/156

(57) ABSTRACT

Disclosed herein are methods and devices for use in early detection of Richter's Syndrome. The methods include sequencing a panel of regions in cell-free DNA molecules and detecting one or more markers that are indicative of Richter's Syndrome.

### CROSS-REFERENCE TO RELATED APPLICATIONS

This application is a continuation of International Application No. PCT/US2022/043647, filed Sep. 15, 2022, which claims priority under 35 U.S.C. § 119 (e) of U.S. Provisional Application Nos. 63/244,625, filed Sep. 15, 2021; and 63/291,213, filed Dec. 17, 2021; the contents of each of which are hereby incorporated by reference into this application in their entireties.

### STATEMENT OF GOVERNMENT SUPPORT

This invention was made with government support under the Grant Nos. K08 CA270085 and CLLP01-P01CA206978 awarded by the National Institutes of Health. The government has certain rights to the invention.

### TECHNICAL FIELD

The present technology generally relates the field of cancer research. More specifically, the present disclosure relates to identification of patients with Richter's Syndrome (RS) and the clinical management of progression of disease.

### BACKGROUND

The following discussion is provided to aid the reader in understanding the disclosure and is not admitted to describe or constitute prior art thereto.

Richter's syndrome (RS) is a highly refractory lymphoma that arises out of the indolent B cell malignancy chronic lymphocytic leukemia (CLL). RS arising from CLL is a major barrier to disease control in CLL patients, and patients with RS have a median overall survival of less than one year, even in modern case series. The genetic basis of RS is poorly understood and its relationship to antecedent CLL remains incompletely characterized. Notable challenges to the genomic study of RS includes those of sample acquisition, the distinction between true tumor events rather than sequence artifacts in archival fixed tissue, and the limitations of available computational techniques for deconvoluting admixtures of CLL and RS DNA within the same biopsy specimen.

RS is traditionally defined by pathology morphologic assessment, which carries a high misdiagnosis rate of up to 20%. Despite an advanced molecular and biologic characterization of CLL through sequencing and experimental studies performed over the past few decades, the systematic identification of RS drivers and understanding of the genetic evolution of CLL to RS remains incompletely examined. Previous studies have been limited by sample cohort size and sensitivity of analysis, but have detected TP53 alterations, NOTCH1 mutations, CDKN2A/B loss/inactivation, MYC amplification in RS, and a potential contributing role of the DNA damage response pathway. However, a more comprehensive study of RS has been thus far limited by the difficulties of sample acquisition in this rapidly progressive malignancy and of availability of paired antecedent CLL, hence posing challenges to performing comparative evolutionary analysis, and in turn, to precisely defining the genetic and transcriptional events underlying transformation. Thus, the transforming defining genetic and transcriptional events remain largely unknown. Moreover, biopsies taken at the time of RS diagnosis typically are comprised of admixtures of RS and CLL cells, and yet established computational tools for the robust in silico deconvolution of such DNA admixtures are still lacking.

Given the admixture of CLL and RS in tissue samples and issues of sampling and sequencing artifact, no study has comprehensively been able to identify the genetic drivers, molecular features, and expression patterns that are unique to RS. RS thus remains poorly understood and there is an urgent need for improved diagnosis, sensitive detection and understanding of the disease biology. Therefore, definitive molecular characterization of RS events are of great interest for improving the diagnosis of RS, impacting clinical management, and providing novel insights into RS disease biology.

### SUMMARY

In one embodiment, the disclosure is directed to a method of detecting a mutation in a sample in which the method comprises obtaining a biological sample from a subject, isolating a nucleic acid sample from the biological sample, and detecting a mutation in at least one of TP53, NOTCH1, IRF2BP2, DNMT3A, SRSF1, EZH2, CCND3, TET2, IRF8, MYC, PIM1, B2M, and PRDM1. The subject may have been diagnosed with or suspected of having chronic lymphocytic leukemia (CLL). The nucleic acid sample may comprise RNA or DNA, with the latter being cell-free DNA (cfDNA). The biological sample can be blood, serum, plasma, urine, and/or saliva. According to the method, the mutation may be detected by nucleic acid sequencing, RT-qPCR, RT-PCR, RNA-seq, Northern blotting, Serial Analysis of Gene Expression (SAGE), or DNA or RNA microarray. The mutation may be detected in at least one of IRF2BP2, DNMT3A, SRSF1, and EZH2.

In another embodiment, the disclosure is directed to a method of distinguishing RS from CLL comprising obtaining a biological sample from a subject with CLL, detecting the presence or absence of a mutation in at least one driver of RS selected from the group consisting of IRF2BP2, DNMT3A, SRSF1, EZH2, B2M, IRF8, PIM1, HIST1H2AC, PRDM1, CCND3, and TET2, wherein the subject has or will develop RS if a mutation is detected in at least one driver of RS and the subject is unlikely to develop RS if a mutation is not detected in at least one driver of RS.

In another embodiment, the disclosure is directed to a method of distinguishing RS from CLL comprising obtaining a biological sample from a subject with CLL, detecting the presence or absence of a mutation in at least one driver of RS selected from the group consisting of IRF2BP2, DNMT3A, SRSF1, EZH2, B2M, IRF8, PIM1, HIST1H2AC, PRDM1, CCND3, and TET2, or at least one genomic alteration selected from the group consisting of amp(9p24), del(16q12), del(18q22), and amp(7q21.2), del(1p), amp(11q), amp(1q23), whole genome doubling (WGD), amp(18q21.33), amp(16.23.2), amp(6p22.1), del(9p), del(9q), and amp(7p).

In yet another embodiment, the disclosure is directed to a method of diagnosing Richter's syndrome in a subject comprising: providing a sample comprising cell-free DNA (cfDNA) molecules from the subject; sequencing at least a portion of the cfDNA; and identifying any mutation in one or more of the following genes: IRF2BP2, DNMT3A, SRSF1, EZH2, B2M, IRF8, PIM1, HIST1H2AC, PRDM1, CCND3, and TET2, wherein the mutation in one or more of IRF2BP2, DNMT3A, SRSF1, EZH2, B2M, IRF8, PIM1, HIST1H2AC, PRDM1, CCND3, and TET2; or the presence of at least one genomic alteration selected from the group consisting of amp(9p24), del(16q12), del(18q22), amp(7q21.2), del(1p), amp(11q), amp(1q23), whole genome doubling (WGD), amp(18q21.33), amp(16.23.2), amp(6p22.1), del(9p), del(9q), and amp(7p), wherein identification of a mutation or genomic alteration is indicative of the subject having Richter's syndrome. The cfDNA molecules may be derived from blood, serum, or plasma. The NOTCH1 mutation may be a 3′UTR mutation, and the mutations may be detected in at least one of IRF2BP2, DNMT3A, SRSF1, and EZH2.

In yet another embodiment, the disclosure is directed to a method for distinguishing RS from CLL, comprising: obtaining a biological sample from a subject with CLL or suspected of having CLL; detecting a gene alteration and a structural variation in the biological sample; and determining that the subject has or will develop RS (i) when the gene alteration comprises loss of TP53 and the structural variation comprises copy number gain of chromosome 1p23 or whole genome doubling; (ii) when the gene alteration comprises both loss of TP53 and/or a NOTCH1 mutation, and the structural variation comprises deletion of chromosome 1p (del(1p)) or deletion of chromosome 2q37(del(2q37)); or (iii) when the gene alteration comprises a NOTCH1 mutation and/or a SPEN mutation without concurrent loss of TP53, and the structural variation comprises absence of deletion of chromosome 13q (del(13q)), and presence of trisomy 12 (tri(12)). In some embodiments, the loss of TP53 occurs through TP53 mutation or through deletion of chromosome 17p (del(17p)). In some embodiments, the NOTCH1 mutation is a 3′UTR mutation.

Another aspect of the disclosure is directed to a method of detecting Richter's Syndrome (RS) subtypes, comprising: obtaining a biological sample from a subject diagnosed with or suspected of having RS, detecting the presence or absence of at least one genomic alteration selected from the group consisting of del(1p), del 9(p), amp(8q24.21), HIST1H1E mutation, del(19p13.3), or whole genome duplication (WGD), tri(12), SPEN mutation, KRAS mutation, del(17p), del(14q32.11), del(9q), NOTCH1 mutation, IRF2BP2 mutation, TP53 mutation, del(15q15.1), amp(16q23.2), del(2q37.1), SF3B1 mutation, EGR2 mutation, del(13q14.2), IRF8 mutation, PIM1 mutation, amp(7p), del(16q12.1), del(1p35.3), and del(18q22.2); and determining the RS subtype based on the at least one genomic alteration, wherein detection of at least one of del(1p), del 9(p), amp(8q24.21), HIST1H1E mutation, del(19p13.3), or whole genome duplication (WGD) indicates RS subtype 1; wherein detection of at least one of tri(12), SPEN mutation, or KRAS mutation indicates RS subtype 2; wherein detection of at least one of del(17p), del(14q32.11), del(9q), NOTCH1 mutation, IRF2BP2 mutation, TP53 mutation, del(15q15.1), amp(16q23.2), or del(2q37.1) indicates RS subtype 3; wherein detection of at least one of SF3B1 mutation, EGR2 mutation, del(13q14.2), or IRF8 mutation indicates RS subtype 4; and wherein detection of at least one of PIM1 mutation, amp(7p), del(16q12.1), del(1p35.3), or del(18q22.2) indicates RS subtype 5.

Another aspect of the disclosure is directed to a processor programmed to perform: i) detecting at least one mutation in at least one gene selected from the group consisting of IRF2BP2, DNMT3A, SRSF1, EZH2, B2M, IRF8, PIM1, HIST1H2AC, PRDM1, CCND3, and TET2 and/or at least one genomic alteration selected from the group consisting of amp(9p24), del(16q12), del(18q22), amp(7q21.2), del(1p), amp(11q), amp(1q23), whole genome doubling (WGD), amp(18q21.33), amp(16.23.2), amp(6p22.1), del(9p), del(9q), and amp(7p); in a sequence data from a subject; and ii) generating a report to a medical professional comprising the indication of whether the subject is suffering from chronic lymphocytic leukemia (CLL) or Richter Syndrome (RS) to inform a decision on treatment.

Another aspect of the disclosure is directed to a computer-readable storage device, comprising instructions to perform: i) detecting at least one mutation in at least one gene selected from the group consisting of IRF2BP2, DNMT3A, SRSF1, EZH2, B2M, IRF8, PIM1, HIST1H2AC, PRDM1, CCND3, and TET2 and/or at least one genomic alteration selected from the group consisting of amp(9p24), del(16q12), del(18q22), amp(7q21.2), del(1p), amp(11q), amp(1q23), whole genome doubling (WGD), amp(18q21.33), amp(16.23.2), amp(6p22.1), del(9p), del(9q), and amp(7p) in a sequence data from a subject; and ii) generating a report to a medical professional comprising the indication of whether the subject is suffering from chronic lymphocytic leukemia (CLL) or Richter Syndrome (RS) to inform a decision on treatment.

Another aspect of the disclosure is directed to a processor programmed to perform: detecting the presence or absence of at least one genomic alteration selected from the group consisting of del(1p), del 9(p), amp(8q24.21), HIST1H1E mutation, del(19p13.3), or whole genome duplication (WGD), tri(12), SPEN mutation, KRAS mutation, del(17p), del(14q32.11), del(9q), NOTCH1 mutation, IRF2BP2 mutation, TP53 mutation, del(15q15.1), amp(16q23.2), del(2q37.1), SF3B1 mutation, EGR2 mutation, del(13q14.2), IRF8 mutation, PIM1 mutation, amp(7p), del(16q12.1), del(1p35.3), and del(18q22.2); and determining the RS subtype based on the at least one genomic alteration, wherein detection of at least one of del(1p), del 9(p), amp(8q24.21), HIST1H1E mutation, del(19p13.3), or whole genome duplication (WGD) indicates RS subtype 1; wherein detection of at least one of tri(12), SPEN mutation, or KRAS mutation indicates RS subtype 2; wherein detection of at least one of del(17p), del(14q32.11), del(9q), NOTCH1 mutation, IRF2BP2 mutation, TP53 mutation, del(15q15.1), amp(16q23.2), or del(2q37.1) indicates RS subtype 3; wherein detection of at least one of SF3B1 mutation, EGR2 mutation, del(13q14.2), or IRF8 mutation indicates RS subtype 4; and wherein detection of at least one of PIM1 mutation, amp(7p), del(16q12.1), del(1p35.3), or del(18q22.2) indicates RS subtype 5.

Another aspect of the disclosure is directed to a computer-readable storage device, comprising instructions to perform: detecting the presence or absence of at least one genomic alteration selected from the group consisting of del(1p), del 9(p), amp (8q24.21), HIST1H1E mutation, del(19p13.3), or whole genome duplication (WGD), tri(12), SPEN mutation, KRAS mutation, del(17p), del(14q32.11), del(9q), NOTCH1 mutation, IRF2BP2 mutation, TP53 mutation, del(15q15.1), amp(16q23.2), del(2q37.1), SF3B1 mutation, EGR2 mutation, del(13q14.2), IRF8 mutation, PIM1 mutation, amp(7p), del(16q12.1), del(1p35.3), and del(18q22.2); and determining the RS subtype based on the at least one genomic alteration, wherein detection of at least one of del(1p), del 9(p), amp (8q24.21), HIST1H1E mutation, del(19p13.3), or whole genome duplication (WGD) indicates RS subtype 1; wherein detection of at least one of tri(12), SPEN mutation, or KRAS mutation indicates RS subtype 2; wherein detection of at least one of del(17p), del(14q32.11), del(9q), NOTCH1 mutation, IRF2BP2 mutation, TP53 mutation, del (15q15.1), amp(16q23.2), or del(2q37.1) indicates RS subtype 3; wherein detection of at least one of SF3B1 mutation, EGR2 mutation, del(13q14.2), or IRF8 mutation indicates RS subtype 4; and wherein detection of at least one of PIM1 mutation, amp(7p), del(16q12.1), del(1p35.3), or del(18q22.2) indicates RS subtype 5.

### BRIEF DESCRIPTION OF THE DRAWINGS

FIGS. 1A-1D. (A) Genetic mutations in clonal related cases for RS and CLL. (B) Genetic mutations in clonal unrelated cases for RS and CLL. (C) Computational schema for deciphering CLL and RS clones within RS biopsy samples. (D) Labeled sample phylogenetic tree with associated sample cancer cell fraction (CCF) plot.

FIGS. 2A-2K. (A) RS somatic alterations in circulating cell-free DNA (cfDNA) at the time of RS diagnosis. (B) Plasma chromosome somatic alterations in circulating cell-free DNA (cfDNA) months before RS diagnosis. (C)-(D) GISTIC2.0 plots showing arm level (right panel) and focal (left panel) amplifications (C) and deletions (D) for RS samples in the combined discovery and validation cohorts (n=97). (E) Frequencies of somatic alterations in CLL clones from related RS cases (n=45, dark bars) compared to CLL driver frequencies using 2 sided exact binomial test with Benjamini-Hochberg multiple hypothesis testing correction. (F) RS somatic alteration frequencies (dark, rightmost bars) compared to DLBCL event frequencies (light, left two bars) from DLBCL cohorts using 2 sided exact binomial test with Benjamini-Hochberg multiple test correction. (G)-(H), Evolution of RS from CLL showing clonal composition and absolute tumor burden over time based on serial sampling for two patients. Left panel-a phylogenetic tree with associated driver events. (Magenta square, RS clones). Right panel-relative abundance of CLL in peripheral blood by white blood cell count (1000 cells/microliter) (top) and relative abundance of RS in bottom plot (by PET/CT scan tumor metrics) with clonal evolution dynamics. Pie charts reflect composition of each sampling timepoint. (dotted line, sampling time; Top bar, treatment history; PB, peripheral blood; BM, bone marrow). (I) Sankey plot showing trajectories from CLL driver to acquired RS driver. Only driver pairs with at least 4 co-occurrences across the cohort are displayed and tested for statistical significance. * denotes P<0.05 (Fisher's exact test) and Q<0.4. (J) Pathways altered in CLL transformation to RS include CLL phase alterations and new drivers identified in RS. sSNV (top shading) and sCNA (bottom shading). (K) Trees depicting clonal evolution of CLL to RS in seven select patients who developed RS on novel agents. Recurrent RS drivers indicated in bold.

FIGS. 3A-3D. Molecular mechanisms underlying transformation to RS. (A) Genomic classification of Richter's syndrome. For 52 patients (columns), 5 patterns of RS are depicted with respective somatic mutations and copy number alterations (rows) clustered according to the related pathways. Samples are annotated for prior treatments (chemoimmunotherapy, novel agent, and no prior therapy), IGHV status (mutated, unmutated, unknown), clonal relationship (related, unrelated) and the presence of whole genome doubling. For each sample, WES mutational signatures according to the Catalogue of Somatic Mutations in Cancer are indicated (see legend) and RS clonal mutations are shown (top). Event frequencies are indicated as bars on the right side for each alteration. (B) WGS signatures by RS subtype. (C) Overall survival (OS) according to Richter genomic subtype. Kaplan-Meier curves for each subtype according to legends. P value is from log-rank (Mantel Cox) testing (D) Overall survival according to the RS genomic pattern. Kaplan-Meier curves for each subtype according to legends. P value is from log-rank (Mantel Cox) testing.

FIGS. 4A-4D. (A) Volcano plot of transcript expression changes in RS compared to CLL. Bigger dots denote select relevant transcripts (Left dots include: CD79A, PAX5, ITGB1, CD37 and LYN; Right dots include: POLQ, AURKA, AURKB, XRCC2, PLK1, E2F7, PLK4, EZH2, BRCA1, CDK1, CDK2, POLE2, E2F8, TERT, AICDA, KIF14, and KIF18B). (B) Schema for assignment of copy number changes to single-cells to enable identification of CLL vs RS cells. (C) Heatmap representation of differential regulated genes between clusters. Single-cell data shows transcriptional differences between RS and CLL and highlights intermediate states. (D) Patient 10: Phylogenetic tree showing clonal structure of RS from WES data (left) and UMAP visualization of RS and CLL single-cells (middle). Heatmap representation of differential regulated genes between clusters (right).

FIGS. 5A-5G. cfDNA isolated from plasma of RS patients shows evidence of transformation. (A) Schema showing how RS specific DNA events can be identified separately from cell-free DNA and different from circulating CLL cells. (B) cfDNA in RS Pt 38 shows WGD of clonally unrelated RS, which is not seen in circulating CLL disease at time of diagnosis. (C) Chromothripsis is observed in cfDNA of RS patients, as demonstrated by plotting the difference between copy number state changes across the genome (Pt 32 top, Pt 5 bottom). (D) Allele frequencies for RS (dark) and CLL (light) mutations found in RS WES sample (bottom) and RS plasma sample cfDNA WES (top) for patient 38 (top panel) and patient 8 (bottom panel). (E) Plasma from patients shows early detection of RS. Pt 5 (top) shows RS-related WGD and chromothripsis fragmentation 162 days prior to RS diagnosis, which is not seen in corresponding co-sampled CLL cells. Plasma from Pt 20 (bottom panel) examined 181 days prior to RS shows RS-related WGD and sSCNVs which are not seen in co-sampled CLL or in lymph node biopsy taken from prior week. (F) sCNAs become detectable prior to post-transplant relapse in Pt 112, as seen by plot of fraction genome altered and corresponding cfDNA samples showing emergence of new sCNVs despite continued remission of circulating and marrow CLL. (G) Metrics of RS in cfDNA are plotted for RS samples leading up to diagnosis. Y axis is fragment genome altered, scale shows presence of chromothripsis, square represents whole genome doubled (WGD) sample and circle outline indicates samples for which RS mutations were detected on WES of cfDNA.

FIGS. 6A-6X. Putative RS driver genes. (A)-(X), individual protein mutation maps for selected putative Richter drivers, showing gene mutation subtype (for example, missense, truncating, inframe, splice, fusion or other mutations), position and evidence of mutational hotspots. Panels were generated by using the cBioPortal for Cancer Genomics tool.

FIG. 7. Block diagram of the system in accordance with the aspects of the disclosure. CPU: Central Processing Unit (“processor”).

FIG. 8 is a block diagram of a computing environment for realizing the systems and methods according to embodiments of the subject matter disclosed herein.

FIG. 9 is a method flow chart illustrating an exemplary computer-based method for establishing a trained machine learning model and updating the trained model according to embodiments of the subject matter disclosed herein.

FIG. 10 is a method flow chart illustrating an exemplary computer-based method for utilizing a trained machine learning model according to embodiments of the subject matter disclosed herein.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

Unless the context indicates otherwise, it is specifically intended that the various features of the invention described herein can be used in any combination. Moreover, the disclosure also contemplates that in one or more embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

### Definitions

As used herein, “about” will be understood by persons of ordinary skill in the art and will vary to some extent depending upon the context in which it is used. If there are uses of the term which are not clear to persons of ordinary skill in the art, given the context in which it is used, such as before a numerical designation, e.g., temperature, time, amount, and concentration, including range, indicates approximations which may vary by (+) or (−) 10%, 5% or 1%.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein may be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, the phrase “at least one” of a group of listed items includes one, two, three, four, five, six, or more or all of the group of listed items.

The expression “comprising” means “including, but not limited to.” For example, compositions and methods include the recited elements, but do not exclude others. “Consisting essentially of” shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

As used herein, “treating” or “treatment” of a disease in a patient refers to (1) preventing the symptoms or disease from occurring in an animal that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, the term treatment excludes prevention or prophylaxis.

As used herein, the term “subject” is used interchangeably with “patient,” and indicates a mammal, or a human, ovine, bovine, feline, canine, equine, simian, etc. Non-human animals subject to diagnosis or treatment include, for example, simians, murine, such as, rat, mice, canine, leporid, livestock, sport animals, and pets. In one or more embodiments, the subject is a human.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks. Consistent with this definition and as used herein, the term “therapeutically effective amount” is an amount sufficient to treat a specified disorder or disease or alternatively to obtain a pharmacological response.

“Pharmaceutically acceptable” means in the present description being useful in preparing a pharmaceutical composition that is generally safe, non-toxic and neither biologically nor otherwise undesirable and includes being useful for veterinary use as well as human pharmaceutical use.

The term “memory” as used herein comprises program memory and working memory. The program memory may have one or more programs or software modules. The working memory stores data or information used by the CPU in executing the functionality described herein.

The term “processor” may include a single core processor, a multi-core processor, multiple processors located in a single device, or multiple processors in wired or wireless communication with each other and distributed over a network of devices, the Internet, or the cloud. Accordingly, as used herein, functions, features or instructions performed or configured to be performed by a “processor”, may include the performance of the functions, features or instructions by a single core processor, may include performance of the functions, features or instructions collectively or collaboratively by multiple cores of a multi-core processor, or may include performance of the functions, features or instructions collectively or collaboratively by multiple processors, where each processor or core is not required to perform every function, feature or instruction individually. The processor may be a CPU (central processing unit). The processor may comprise other types of processors such as a GPU (graphical processing unit). In other aspects of the disclosure, instead of or in addition to a CPU executing instructions that are programmed in the program memory, the processor may be an ASIC (application-specific integrated circuit), analog circuit or other functional logic, such as a FPGA (field-programmable gate array), PAL (Phase Alternating Line) or PLA (programmable logic array).

The CPU is configured to execute programs (also described herein as modules or instructions) stored in a program memory to perform the functionality described herein. The memory may be, but not limited to, RAM (random access memory), ROM (read-only memory) and persistent storage. The memory is any piece of hardware that is capable of storing information, such as, for example without limitation, data, programs, instructions, program code, and/or other suitable information, either on a temporary basis and/or a permanent basis.

The present technology provides an in depth genomic characterization of RS and provide a genetic definition for RS with high clinical translation potential. Furthermore, the technology establishes the use of circulating cell-free DNA (cfDNA) as a potential tool for diagnosis and identify novel RS specific driver alterations.

### Methods

The present disclosure is predicated on the finding that RS arises from CLL subclones through distinct mutational trajectories. The present disclosure provides molecular subclassification of RS, including genetic characterization of additional cases, and linking mutational data with clinical outcomes which has the potential to alter clinical classification and prognostication of RS. Through the implementation of advanced genomic analytic approaches and integration of exome, genome and transcriptome data, the present technology identifies the distinct molecular events that precede and define the RS transition, and provides a comprehensive molecular definition of RS-changing from a pathology based description to a molecular classification.

In some embodiments, the cell free DNA is compared to the DNA of circulating PBMC cells from the same sample. In some embodiments, the methods of the instant disclosure are executed using a sample derived from a single blood draw from a subject.

In one aspect, the present disclosure provides a method for identifying a variety of sequence variations associated with Richter's syndrome that may be useful in diagnosis, prognosis, or treatment decisions. Suitable target sequences useful in the methods of this disclosure include, but are not limited to, mutations in the TP53 gene, the NOTCH1 gene, the IRF2BP2 gene, the DNMT3A gene, the SRSF1 gene, the EZH2 gene, the FBXW7 gene, the SPEN gene, the SF3B1 gene, the B2M gene, the IRF8 gene, the PIM1 gene, the GNB1 gene, the XPO1 gene, the HIST1H1E gene, the HIST1H2AC gene, the EGR2 gene, the MGA gene, the CARD11 gene, the KRAS gene, the PRDM1 gene, the ATM gene, the CCND3 gene, the TET2, and the BRAF gene. Target sequences that can be specifically analyzed for sequence variations may be all or part of the particular gene. Sequence mutations can occur anywhere in the gene. Thus, all or part of the specific gene may be evaluated herein.

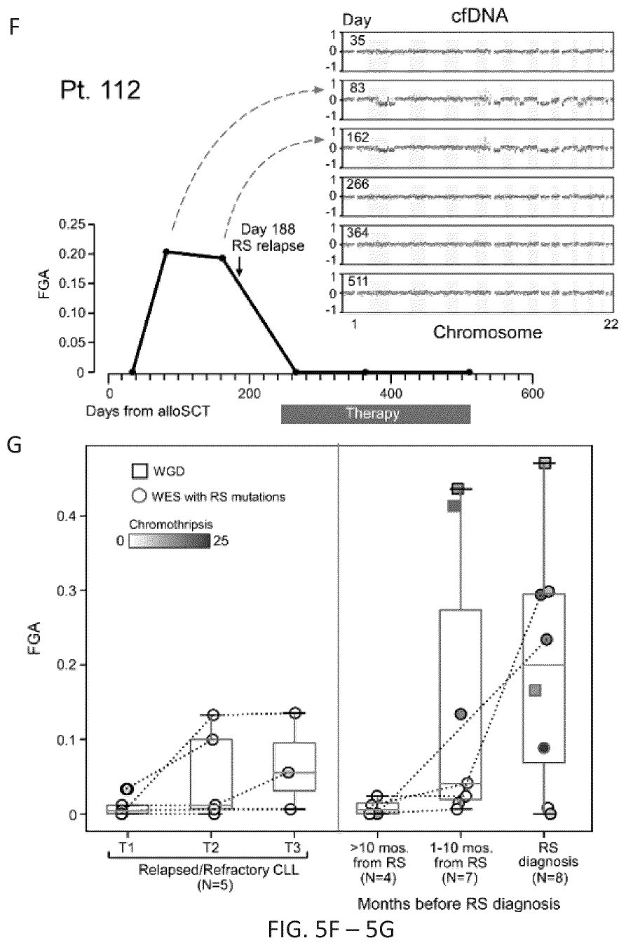

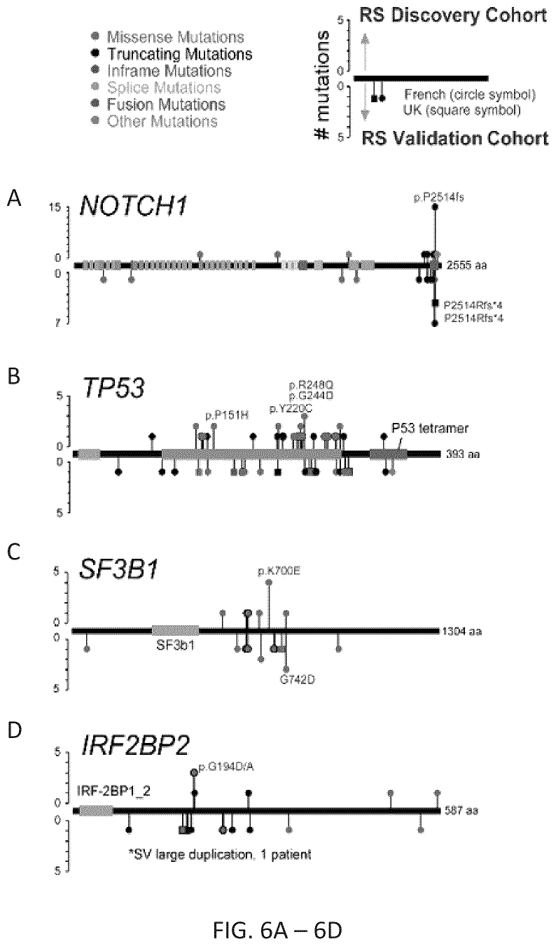

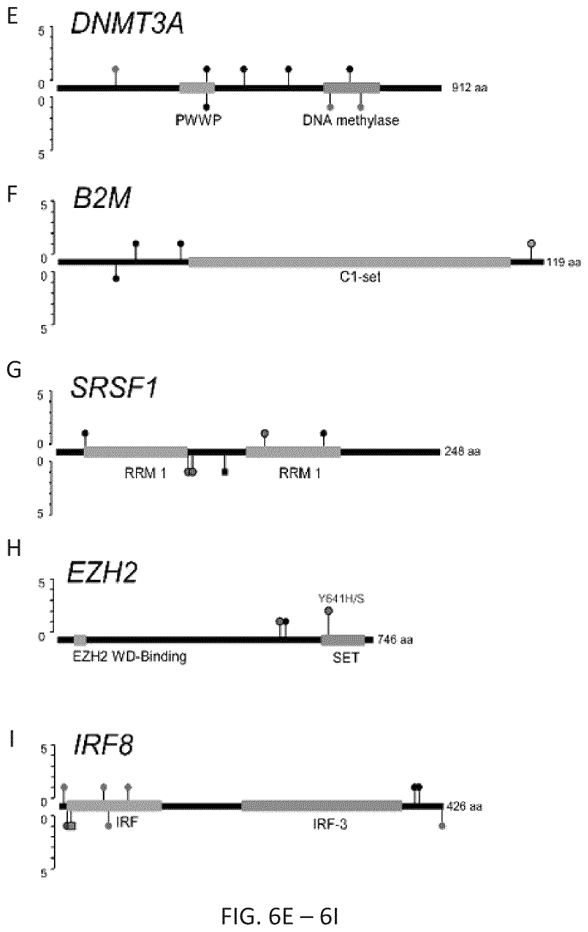

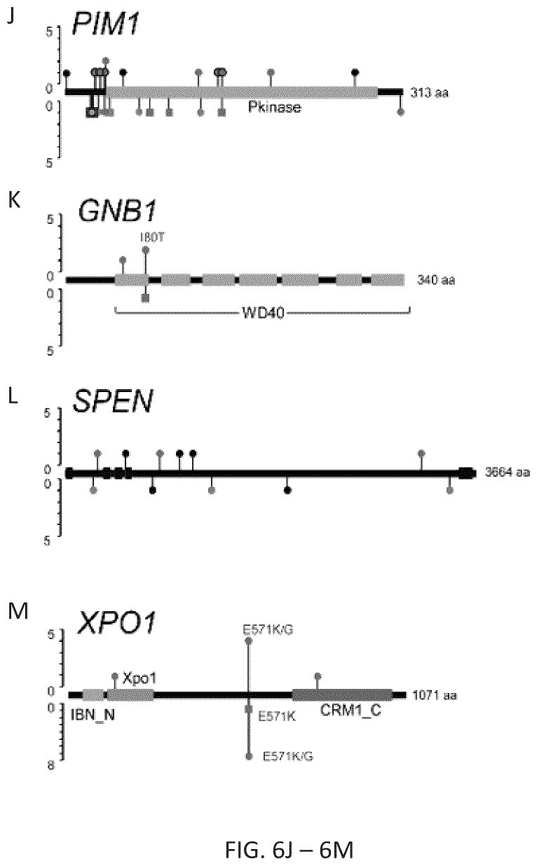

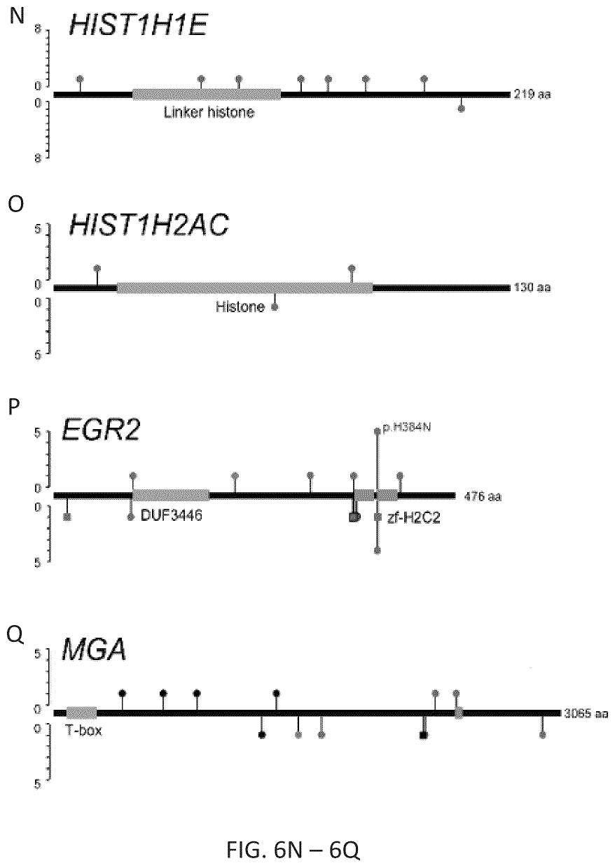

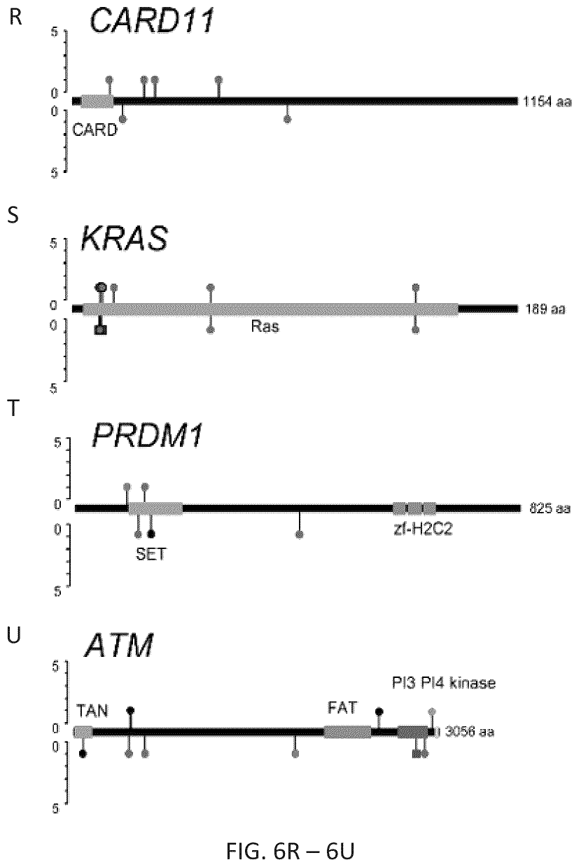

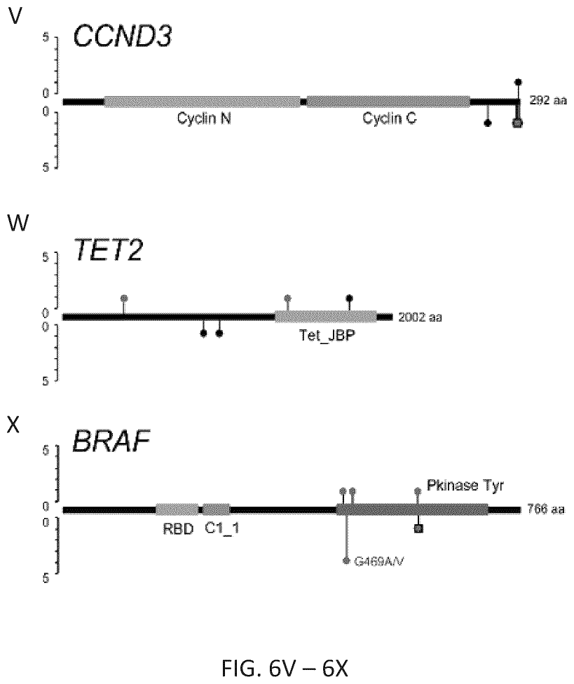

In some embodiments, one or more sequence mutations are identified in all or part of the TP53 gene (Gene ID: 7157), which is one of the most frequently mutated genes in human cancers. In some embodiments, the TP53 mutations include mutations shown in FIG. 6B. In some embodiments, one or more sequence mutations are identified in all or part of the NOTCH1 gene (Gene ID: 4851). In some embodiments, the NOTCH1 mutations include mutations shown in FIG. 6A. In some embodiments, one or more sequence mutations are identified in all or part of the IRF2BP2 gene (Gene ID: 359948). In some embodiments, the IRF2BP2 mutations include mutations shown in FIG. 6D. In some embodiments, one or more sequence mutations are identified in all or part of the DNMT3A gene (Gene ID: 1788). In some embodiments, the DNMT3A mutations include mutations shown in FIG. 6E. In some embodiments, one or more sequence mutations are identified in all or part of the SRSF1 gene (Gene ID: 6426). In some embodiments, the SRSF1 mutations include mutations shown in FIG. 6G. In some embodiments, one or more sequence mutations are identified in all or part of the EZH2 gene. In some embodiments, the EZH2 mutations include mutations shown in FIG. 6H. In some embodiments, one or more sequence mutations are identified in all or part of the FBXW7 gene (Gene ID: 55294). In some embodiments, one or more sequence mutations are identified in all or part of the SPEN gene (Gene ID: 23013). In some embodiments, the SPEN mutations include mutations shown in FIG. 6L. In some embodiments, one or more sequence mutations are identified in all or part of the SF3B1 gene (Gene ID: 23451). In some embodiments, the SF3B1 mutations include mutations shown in FIG. 6C. In some embodiments, one or more sequence mutations are identified in all or part of the B2M gene (Gene ID: 567). In some embodiments, the B2M mutations include mutations shown in FIG. 6F. In some embodiments, one or more sequence mutations are identified in all or part of the IRF8 gene (Gene ID: 3394). In some embodiments, the IRF8 mutations include mutations shown in FIG. 6I. In some embodiments, one or more sequence mutations are identified in all or part of the PIM1 gene (Gene ID: 5292). In some embodiments, the PIM1 mutations include mutations shown in FIG. 6J. In some embodiments, one or more sequence mutations are identified in all or part of the GNB1 gene. In some embodiments, the GNB1 mutations include mutations shown in FIG. 6K. In some embodiments, one or more sequence mutations are identified in all or part of the XPO1 gene (Gene ID: 7514). In some embodiments, the XPO1 mutations include mutations shown in FIG. 6M. In some embodiments, one or more sequence mutations are identified in all or part of the HIST1H1E gene (Gene ID: 3008). In some embodiments, the HIST1H1E mutations include mutations shown in FIG. 6N. In some embodiments, one or more sequence mutations are identified in all or part of the HIST1H2AC gene (ID: 8334). In some embodiments, the HIST1H2AC mutations include mutations shown in FIG. 6O. In some embodiments, one or more sequence mutations are identified in all or part of the EGR2 gene (Gene ID: 1959). In some embodiments, one or more sequence mutations are identified in all or part of the MGA gene (Gene ID: 23269). In some embodiments, the MGA mutations include mutations shown in FIG. 6Q. In some embodiments, one or more sequence mutations are identified in all or part of the CARD11 gene (Gene ID: 84433). In some embodiments, the CARD11 mutations include mutations shown in FIG. 6R. In some embodiments, one or more sequence mutations are identified in all or part of the KRAS gene (Gene ID: 3845). In some embodiments, the KRAS mutations include mutations shown in FIG. 6S. In some embodiments, one or more sequence mutations are identified in all or part of the PRDM1 gene (Gene ID: 639). In some embodiments, the PRDM1 mutations include mutations shown in FIG. 6T. In some embodiments, one or more sequence mutations are identified in all or part of the ATM gene (Gene ID: 472). In some embodiments, the ATM mutations include mutations shown in FIG. 6U. In some embodiments, one or more sequence mutations are identified in all or part of the CCND3 gene (Gene ID: 896). In some embodiments, the CCND3 mutations include mutations shown in FIG. 6V. In some embodiments, one or more sequence mutations are identified in all or part of the TET2 gene (Gene ID: 54790). In some embodiments, the TET2 mutations include mutations shown in FIG. 6W. In some embodiments, one or more sequence mutations are identified in all or part of the BRAF gene (Gene ID: 673). In some embodiments, the BRAF mutations include mutations shown in FIG. 6X.

In one aspect, the present disclosure provides methods for identifying RS-specific changes, including, but not limited to, EZH2 mutations-both clonal hotspot mutations seen in clonal unrelated cases and EZH2 frameshift in clonal related cases.

In another aspect, the present disclosure provides a method of detecting a mutation in a sample, comprising obtaining a biological sample from a subject, isolating a nucleic acid sample from the biological sample, and detecting a mutation in at least one of TP53, NOTCH1, IRF2BP2, DNMT3A, SRSF1, EZH2, CCND3, TET2, IRF8, MYC, PIM1, B2M, and PRDM1.

In some embodiments, the subject has been diagnosed with or is suspected of having chronic lymphocytic leukemia (CLL). In some embodiments, the nucleic acid sample comprises RNA or DNA. In some embodiments, the DNA is cell-free DNA (cfDNA).

In some embodiments, the biological sample is blood, plasma, serum, saliva, urine, tears, gastric fluid, digestive fluid, bone marrow, cerebrospinal fluid, stool, semen, vaginal fluid, or liquid extracted from tissue.

In some embodiments, detecting the mutation is performed by nucleic acid sequencing, RT-qPCR, RT-PCR, RNA-seq, Northern blotting, Serial Analysis of Gene Expression (SAGE), or DNA or RNA microarray.

In some embodiments, the mutation is detected in at least one (e.g., at least 1, 2, 3 or all) of IRF2BP2, DNTM3A, SRSF1, and EZH2 genes.

In some embodiments, the method further comprises detecting a mutation in at least one (e.g., at least 1, 2, 3, 4, 5, 6, 7, 8, 9, 10, 11, 12, 13, 14, 15, 16, 17 or all) of SF3B1, B2M, IRF8, PIM1, GNB1, XPO1, HIST1H1E, HISTH2AC, EGR2, MGA, CARD11, KRAS, PRDM1, ATM, CCND3, TET2, and BRAF.

In some embodiments, the method further comprises detecting and/or identifying at least one (e.g., at least 1, 2, 3, 4, 5, 6, 7, 8, 9, 10, 11, 12, 13, 14, 15, 16, 17 or all of the listed) genomic alteration selected from the group consisting of amp(9p24), del(16q12), del(18q22), amp(7q21.2), del(1p), amp(11q), amp(1q23), whole genome doubling (WGD), amp(18q21.33), amp(16.23.2), amp(6p22.1), del(19p13.3), del(12p13.2), del(1q42.13), del(10q24.32), del(1p35.3), del(3p21.31), and del(13q14.2) (amp denotes amplification, del denotes deletion).

In one aspect, the present disclosure provides a method for identifying a variety of genomic alterations associated with Richter's syndrome that may be useful in diagnosis, prognosis, or treatment decisions. Exemplary genomic alterations useful in the methods of this disclosure include, but are not limited to, amp(9p24), del(16q12), del(18q22), amp(7q21.2), del(1p), amp(11q), amp(1q23), whole genome doubling (WGD), amp(18q21.33), amp(16.23.2), amp(6p22.1), del(9p), del(9q), and amp(7p) (amp denotes amplification, del denotes deletion).

In another aspect, the present disclosure provides a method for identifying a mutation associated with Richter's syndrome. Exemplary mutations useful in the methods of this disclosure include, but are not limited to, a mutation in IRF2BP2, DNMT3A, SRSF1, EZH2, B2M, IRF8, PIM1, HIST1H2AC, PRDM1, CCND3, or TET2.

In one aspect, the present disclosure provides a method comprising: a. providing a biological sample (e.g., blood, plasma, serum, saliva, urine, tears, gastric fluid, digestive fluid, bone marrow, cerebrospinal fluid, stool, semen, vaginal fluid, or liquid extracted from tissue) comprising DNA, preferably cell-free DNA (cfDNA) molecules from a subject, wherein the subject does not detectably exhibit Richter's syndrome; b. sequencing cell-free DNA (cfDNA) molecules derived from the cell-free DNA (cfDNA) sample to provide a sequencing panel; wherein the sequencing panel comprises one or more regions from each of a plurality of different genes; and c. analyzing the sequencing panel to identify one or more mutations or genomic alterations disclosed herein in the cfDNA sample.

In one aspect, the present disclosure provides a method for detecting the presence or recurrence of RS in a subject, the method comprising detecting the presence of clonal and/or subclonal mutations from RS, analyzing cfDNA in a sample obtained from the subject and determining whether RS is present or has recurred by detecting the clonal and/or subclonal mutation from the RS in the sample. In another aspect, the present disclosure relates to a method comprising detecting and/or assaying a genetic mutation. The genetic mutation may be a clonal mutation or a subclonal mutation. In some embodiments, the mutation is clonally related to antecedent CLL. In other embodiments, the mutation is clonally unrelated to antecedent CLL.

In one aspect, the present disclosure provides a method comprising detecting the presence of one or more mutations in one or more samples from the subject. In some embodiments, the one or more mutations are identified by screening genes which are identified as drivers in RS, e.g., TP53, NOTCH1, IRF2BP2, DNMT3A, SRSF1, EZH2, FBXW7, the SPEN gene, the SF3B1 gene, the B2M gene, the IRF8 gene, the PIM1 gene, the GNB1 gene, the XPO1 gene, the HIST1H1E gene, the HIST1H2AC gene, the EGR2 gene, the MGA gene, the CARD11 gene, the KRAS gene, the PRDM1 gene, the ATM gene, the CCND3 gene, the TET2, and the BRAF gene.

Another aspect of the disclosure is directed to a method of distinguishing Richter's Syndrome (RS) from chronic lymphocytic leukemia (CLL), comprising obtaining a biological sample from a subject with CLL, detecting the presence or absence of a mutation in at least one driver of RS selected from the group consisting of IRF2BP2, DNMT3A, SRSF1, EZH2, B2M, IRF8, PIM1, HIST1H2AC, PRDM1, CCND3, and TET2 wherein the subject has or will develop RS if a mutation is detected in at least one driver of RS and the subject is unlikely to develop RS if a mutation is not detected in at least one driver of RS.

Another aspect of the disclosure is directed to a method of distinguishing Richter's Syndrome (RS) from chronic lymphocytic leukemia (CLL), comprising: obtaining a biological sample from a subject with CLL, detecting the presence or absence of a mutation in at least one driver of RS selected from the group consisting of TP53, NOTCH1, IRF2BP2, DNTM3A, SRSF1, EZH2, FBXW7, SPEN, SF3B1, B2M, IRF8, PIM1, GNB1, XPO1, HIST1H1E, HIST1H2AC, EGR2, MGA, CARD11, KRAS, PRDM1, ATM, CCND3, TET2, and BRAF, or at least one genomic alteration selected from the group consisting of amp(9p24), del(16q12), del(18q22), amp(7q21.2), del(1p), amp(11q), amp(1q23), whole genome doubling (WGD), amp(18q21.33), amp(16.23.2), amp(6p22.1), del(9p), del(9q), and amp(7p); wherein the subject has or will develop RS if a mutation is detected in at least one driver of RS or at least one genomic alteration is detected, and the subject is unlikely to develop RS if a mutation in at least one driver of RS or a genomic alteration is not detected.

Another aspect of the disclosure is directed to a method of diagnosing Richter's syndrome in a subject, comprising: a. providing a sample comprising cell-free DNA (cfDNA) molecules from a subject; b. sequencing at least a portion of the cfDNA; and c. identifying a mutation in one or more of the following genes: TP53, NOTCH1, IRF2BP2, DNTM3A, SRSF1, EZH2, FBXW7, SPEN, SF3B1, B2M, IRF8, PIM1, GNB1, XPO1, HIST1H1E, HISTH2AC, EGR2, MGA, CARD11, KRAS, PRDM1, ATM, CCND3, TET2, and BRAF; or identifying at least one genomic alteration selected from the group consisting of amp(9p24), del(16q12), del(18q22), amp(7q21.2), del(1p), amp(11q), amp(1q23), whole genome doubling (WGD), amp(18q21.33), amp(16.23.2), amp(6p22.1), del(9p), del(9q), and amp(7p), del(19p13.3), del(12p13.2), del(1q42.13), del(10q24.32), del(1p35.3), del(3p21.31), and del(13q14.2); wherein a mutation in one or more of TP53, NOTCH1, IRF2BP2, DNTM3A, SRSF1, EZH2, FBXW7, SPEN, SF3B1, B2M, IRF8, PIM1, GNB1, XPO1, HIST1H1E, HISTH2AC, EGR2, MGA, CARD11, KRAS, PRDM1, ATM, CCND3, TET2, and BRAF or a genomic alteration is indicative of the subject having Richter's syndrome.

In some embodiments, the cfDNA molecules are derived from blood, plasma, serum, saliva, urine, tears, gastric fluid, digestive fluid, bone marrow, cerebrospinal fluid, stool, semen, vaginal fluid, or liquid extracted from tissue.

In some embodiments, the NOTCH1 mutation is a 3′UTR mutation.

In some embodiments, the mutation is detected in at least one of IRF2BP2, DNTM3A, SRSF1, and EZH2.

Another aspect of the disclosure is directed to a method for distinguishing RS from CLL, comprising: obtaining a biological sample (e.g., blood, plasma, serum, saliva, urine, tears, gastric fluid, digestive fluid, bone marrow, cerebrospinal fluid, stool, semen, vaginal fluid, or liquid extracted from tissue) from a subject with CLL or suspected of having CLL; detecting a gene alteration and a structural variation in the biological sample; and determining that the subject has or will develop RS (i) when the gene alteration comprises loss of TP53 and the structural variation comprises copy number gain of chromosome 1p23 or whole genome doubling; (ii) when the gene alteration comprises both loss of TP53 and/or a NOTCH1 mutation, and the structural variation comprises deletion of chromosome 1p (del(1p)) or deletion of chromosome 2q37(del(2q37)); or (iii) when the gene alteration comprises a NOTCH1 mutation and/or a SPEN mutation without concurrent loss of TP53, and the structural variation comprises absence of deletion of chromosome 13q (del(13q)), and presence of trisomy 12 (tri(12)). In some embodiments, the biological sample comprises cell-free DNA (cfDNA) or genomic DNA.

In some embodiments, the loss of TP53 occurs through TP53 mutation or through deletion of chromosome 17p (del(17p)). In some embodiments, the NOTCH1 mutation is a 3′UTR mutation.

Another aspect of the disclosure is directed to a method for distinguishing RS from CLL, comprising: obtaining a biological sample (e.g., blood, plasma, serum, saliva, urine, tears, gastric fluid, digestive fluid, bone marrow, cerebrospinal fluid, stool, semen, vaginal fluid, or liquid extracted from tissue) from a subject with CLL or suspected of having CLL; detecting a gene alteration and a structural variation in the biological sample; and determining that the subject has or will develop RS (i) when a mutation in at least one driver of RS selected from the group consisting of IRF2BP2, DNMT3A, SRSF1, EZH2, B2M, IRF8, PIM1, HIST1H2AC, PRDM1, CCND3, and TET2, or at least one genomic alteration selected from the group consisting of amp(9p24), del(16q12), del(18q22), amp(7q21.2), del(1p), amp(11q), amp(1q23), whole genome doubling (WGD), amp(18q21.33), amp(16.23.2), amp(6p22.1), del(9p), del(9q), and amp(7p) is detected.

Another aspect of the disclosure is directed to a method of detecting Richter's Syndrome (RS) subtypes, comprising: obtaining a biological sample from a subject diagnosed with or suspected of having RS, detecting the presence or absence of at least one genomic alteration selected from the group consisting of del(1p), del 9(p), amp(8q24.21), HIST1H1E mutation, del(19p13.3), or whole genome duplication (WGD), tri(12), SPEN mutation, KRAS mutation, del(17p), del(14q32.11), del(9q), NOTCH1 mutation, IRF2BP2 mutation, TP53 mutation, del(15q15.1), amp(16q23.2), del(2q37.1), SF3B1 mutation, EGR2 mutation, del(13q14.2), IRF8 mutation, PIM1 mutation, amp(7p), del(16q12.1), del(1p35.3), and del(18q22.2); and determining the RS subtype based on the at least one genomic alteration, wherein detection of at least one of del(1p), del 9(p), amp(8q24.21), HIST1H1E mutation, del(19p13.3), or whole genome duplication (WGD) indicates RS subtype 1; wherein detection of at least one of tri(12), SPEN mutation, or KRAS mutation indicates RS subtype 2; wherein detection of at least one of del(17p), del(14q32.11), del(9q), NOTCH1 mutation, IRF2BP2 mutation, TP53 mutation, del(15q15.1), amp(16q23.2), or del(2q37.1) indicates RS subtype 3; wherein detection of at least one of SF3B1 mutation, EGR2 mutation, del(13q14.2), or IRF8 mutation indicates RS subtype 4; and wherein detection of at least one of PIM1 mutation, amp(7p), del(16q12.1), del(1p35.3), or del(18q22.2) indicates RS subtype 5.

In some embodiments, the methods of the instant disclosure further comprise isolating DNA from circulating peripheral blood mononuclear cells (PBMCs) in the biological sample. In some embodiments, the methods further comprise detecting mutations of the RS-related genes as described herein, and/or RS specific genomic alterations as listed herein in circulating PBMCs from the same subject. In some embodiments, the methods further comprise comparing the mutations in the cfDNA and the DNA from circulating PBMCs. Without being limited to a particular theory, RS comprises two malignancies: CLL and RS. It is believed that the CLL cells reside in the PBMC fraction, therefore, a comparison with the DNA from PMBCs from the same patient would provide what type of mutations or genomic alterations are not in the CLL (sCNAs, clonal structure, chromothripsis, whole genome duplication, as described herein), thereby assisting in the detection and diagnosis of RS.

The present disclosure advantageously presents a technology which provides: (i) sensitive and early clinical diagnostic testing of RS, particularly improved non-invasive methods that can readily distinguish RS from aggressive CLL, and (ii) sensitive tracking of clinical response and relapse without need for biopsy sampling, given the typical tissue localization of disease. The technology further provides classification of different types of RS with prognostic significance that may inform clinical decision making or be used in clinical trials. The technology also provides clinical definition to enhance pathology morphologic diagnosis, which would allow RS to be more easily recognized and diagnosed. The technology can be used to provide a genetic toolbox to improve diagnosis through targeted tumor sequencing and cfDNA studies. cfDNA approaches rely on easily obtained venipuncture plasma samples and thus are agnostic to tumor site and overcome issues posed by inadequate sampling and biopsy targeting since they do not require a pure tumor cell population. The improved methods for detecting response and remission described herein may guide subsequent therapeutic decisions, such as need for consolidation therapy, and improve the ease of differentiating between persistent indolent CLL and RS recurrence/persistence without the need for invasive biopsies.

In some embodiments, protein or polypeptide expression levels of the disclosed biomarkers may be detected via Western blotting, enzyme-linked immunosorbent assays (ELISA), dot blotting, immunohistochemistry, immunofluorescence, immunoprecipitation, immunoelectrophoresis, or mass-spectrometry.

Additionally or alternatively, in some embodiments, polynucleotides encoding the disclosed biomarkers may be detected by RT-qPCR, RT-PCR, RNA-seq, Northern blotting, Serial Analysis of Gene Expression (SAGE), or DNA or RNA microarrays. The starting material for detection of polynucleotides encoding the disclosed biomarkers may be genomic DNA, cDNA, RNA or mRNA. Nucleic acid amplification can be linear or exponential. Specific variants or mutations may be detected by the use of amplification methods with the aid of oligonucleotide primers or probes designed to interact with or hybridize to a particular target sequence in a specific manner, thus amplifying only the target variant. Primers and probes may also include a detectable label or a plurality of detectable labels. The detectable label associated with the probe can generate a detectable signal directly. Additionally, the detectable label associated with the probe can be detected indirectly using a reagent, wherein the reagent includes a detectable label, and binds to the label associated with the probe.

In some embodiments, detectably labeled primers or probes can be used in hybridization assays including, but not limited to Northern blots, Southern blots, microarray, dot or slot blots, and in situ hybridization assays such as fluorescent in situ hybridization (FISH) to detect a target nucleic acid sequence within a biological sample. Detectably labeled probes can also be used to monitor the amplification of a target nucleic acid sequence. In some embodiments, detectably labeled probes present in an amplification reaction are suitable for monitoring the amount of amplicon(s) produced as a function of time. Examples of such probes include, but are not limited to, 5′-exonuclease assay (TAQMAN® probes described herein (see also U.S. Pat. No. 5,538,848) various stem-loop molecular beacons (see for example, U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, for example, Kubista et al., 2001, SPIE 4264:53-58), non-FRET probes (see, for example, U.S. Pat. No. 6,150,097), Sunrise®/Amplifluor™ probes (U.S. Pat. No. 6,548,250), stem-loop and duplex Scorpion probes (Solinas et al., 2001, Nucleic Acids Research 29: E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem. Res. Toxicol. 15:118-126; and Yu et al., 2001, J. Am. Chem. Soc 14:11155-11161. In some embodiments, the detectable label is a fluorophore. Suitable fluorescent moieties include but are not limited to the following fluorophores working individually or in combination: 4-acetamido-4′-isothiocyanatostilbene-2,2′disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl) maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120), 7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)amino fluorescein (DTAF), 2′, 7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescem (TET); fiuorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSYR 21; QSY® 35 (Molecular Probes); Reactive Red 4 (Cibacron®Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; tetramethyl rhodamine isothiocyanate (TRITC); and VIC®. Detector probes can also comprise sulfonate derivatives of fluorescenin dyes with S03 instead of the carboxylate group, phosphoramidite forms of fluorescein, phosphoramidite forms of CY 5 (commercially available for example from Amersham).

Primers or probes may be designed to selectively hybridize to any portion of a nucleic acid sequence encoding a polypeptide biomarkers of the present disclosure. Methods for preparing the primers or probes have been well developed in the art.

### Processors and Computer-Readable Storage Device

Various aspects of the present disclosure may be embodied as a program, software, or computer instructions embodied or stored in a computer or machine usable or readable medium, or a group of media which causes the computer or machine to perform the steps of the method when executed on the computer, processor, and/or machine. A program storage device readable by a machine, e.g., a computer readable medium, tangibly embodying a program of instructions executable by the machine to perform various functionalities and methods described in the present disclosure is also provided.

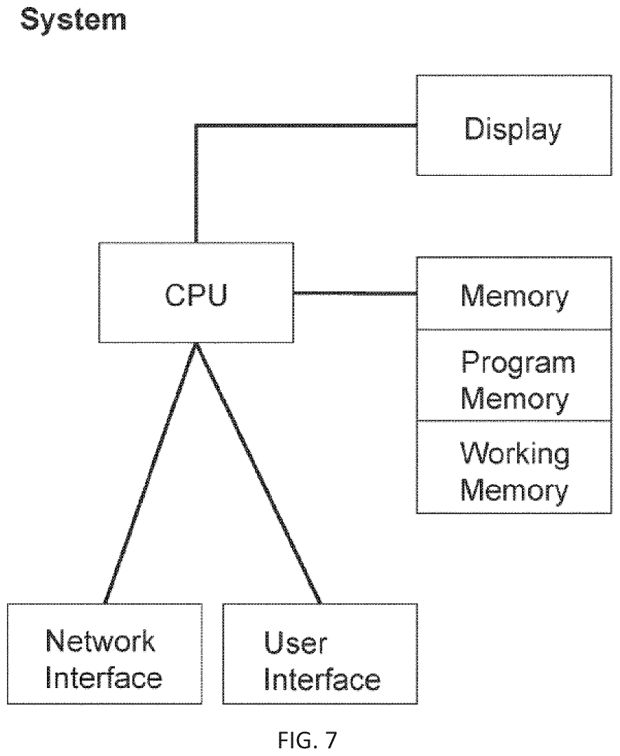

In some embodiments, the present disclosure includes a system comprising a CPU, a display, a network interface, a user interface, a memory, a program memory and a working memory, where the system is programmed to execute a program, software, or computer instructions directed to methods or processes of the instant disclosure (See FIG. 7).

An aspect of the disclosure is directed to a processor programmed to perform: i) detecting at least one mutation in at least one gene selected from the group consisting of IRF2BP2, DNMT3A, SRSF1, EZH2, B2M, IRF8, PIM1, HIST1H2AC, PRDM1, CCND3, and TET2 and/or at least one genomic alteration selected from the group consisting of amp(9p24), del(16q12), del(18q22), amp(7q21.2), del(1p), amp(11q), amp(1q23), whole genome doubling (WGD), amp(18q21.33), amp(16.23.2), amp(6p22.1), del(9p), del(9q), and amp(7p) in a sequence data from a subject; and ii) generating a report to a medical professional comprising the indication of whether the subject is suffering from chronic lymphocytic leukemia (CLL) or Richter Syndrome (RS) to inform a decision on treatment.

In some embodiments, the detecting is achieved by comparing the sequence data of the subject with a reference genome sequence. In some embodiments, the generating a report is achieved by updating a graphical user interface. In some embodiments, the graphical user interface is a monitor.

In some embodiments, the sequence data is from cell-free DNA (cfDNA). In some embodiments, the sequence data is generated by whole genome sequencing, e.g., next generation sequencing.

Another aspect of the disclosure is directed to a computer-readable storage device, comprising instructions to perform: i) detecting at least one mutation in at least one gene selected from the group consisting of IRF2BP2, DNMT3A, SRSF1, EZH2, B2M, IRF8, PIM1, HIST1H2AC, PRDM1, CCND3, and TET2 and/or at least one genomic alteration selected from the group consisting of amp(9p24), del(16q12), del(18q22), amp(7q21.2), del(1p), amp(11q), amp(1q23), whole genome doubling (WGD), amp(18q21.33), amp(16.23.2), amp(6p22.1), del(9p), del(9q), and amp(7p) in a sequence data from a subject; and ii) generating a report to a medical professional comprising the indication of whether the subject is suffering from chronic lymphocytic leukemia (CLL) or Richter Syndrome (RS) to inform a decision on treatment.

In some embodiments, the detecting is achieved by comparing the sequence data of the subject with a reference genome sequence. In some embodiments, the generating a report is achieved by updating a graphical user interface. In some embodiments, the graphical user interface is a monitor.

In some embodiments, the sequence data is from cell-free DNA (cfDNA). In some embodiments, the sequence data is generated by whole genome sequencing, e.g., next generation sequencing.

Another aspect of the disclosure is directed to a processor programmed to perform: detecting the presence or absence of at least one genomic alteration selected from the group consisting of del(1p), del 9(p), amp(8q24.21), HIST1H1E mutation, del(19p13.3), or whole genome duplication (WGD), tri(12), SPEN mutation, KRAS mutation, del(17p), del(14q32.11), del(9q), NOTCH1 mutation, IRF2BP2 mutation, TP53 mutation, del(15q15.1), amp(16q23.2), del(2q37.1), SF3B1 mutation, EGR2 mutation, del(13q14.2), IRF8 mutation, PIM1 mutation, amp(7p), del(16q12.1), del(1p35.3), and del(18q22.2); and determining the RS subtype based on the at least one genomic alteration, wherein detection of at least one of del(1p), del 9(p), amp(8q24.21), HIST1H1E mutation, del(19p13.3), or whole genome duplication (WGD) indicates RS subtype 1; wherein detection of at least one of tri(12), SPEN mutation, or KRAS mutation indicates RS subtype 2; wherein detection of at least one of del(17p), del(14q32.11), del(9q), NOTCH1 mutation, IRF2BP2 mutation, TP53 mutation, del(15q15.1), amp(16q23.2), or del(2q37.1) indicates RS subtype 3; wherein detection of at least one of SF3B1 mutation, EGR2 mutation, del(13q14.2), or IRF8 mutation indicates RS subtype 4; and wherein detection of at least one of PIM1 mutation, amp(7p), del(16q12.1), del(1p35.3), or del(18q22.2) indicates RS subtype 5; and generating a report to a medical professional comprising the prognosis of the subject to inform a decision on treatment. In some embodiments, subjects having subtype 2 or subtype 4 RS have better prognosis (e.g., longer lifespan, fewer symptoms) than subjects having subtype 1, subtype 3 or subtype 5 RS.

In some embodiments, the detecting is achieved by comparing the sequence data of the subject with a reference genome sequence. In some embodiments, the generating a report is achieved by updating a graphical user interface. In some embodiments, the graphical user interface is a monitor.

In some embodiments, the sequence data is from cell-free DNA (cfDNA). In some embodiments, the sequence data is generated by whole genome sequencing, e.g., next generation sequencing.

Another aspect of the disclosure is directed to a computer-readable storage device, comprising instructions to perform: detecting the presence or absence of at least one genomic alteration selected from the group consisting of del(1p), del 9(p), amp (8q24.21), HIST1H1E mutation, del(19p13.3), or whole genome duplication (WGD), tri(12), SPEN mutation, KRAS mutation, del(17p), del(14q32.11), del(9q), NOTCH1 mutation, IRF2BP2 mutation, TP53 mutation, del(15q15.1), amp(16q23.2), del(2q37.1), SF3B1 mutation, EGR2 mutation, del(13q14.2), IRF8 mutation, PIM1 mutation, amp(7p), del(16q12.1), del(1p35.3), and del(18q22.2); and determining the RS subtype based on the at least one genomic alteration, wherein detection of at least one of del(1p), del 9(p), amp (8q24.21), HIST1H1E mutation, del(19p13.3), or whole genome duplication (WGD) indicates RS subtype 1; wherein detection of at least one of tri(12), SPEN mutation, or KRAS mutation indicates RS subtype 2; wherein detection of at least one of del(17p), del(14q32.11), del(9q), NOTCH1 mutation, IRF2BP2 mutation, TP53 mutation, del (15q15.1), amp(16q23.2), or del(2q37.1) indicates RS subtype 3; wherein detection of at least one of SF3B1 mutation, EGR2 mutation, del(13q14.2), or IRF8 mutation indicates RS subtype 4; and wherein detection of at least one of PIM1 mutation, amp(7p), del(16q12.1), del(1p35.3), or del(18q22.2) indicates RS subtype 5; and generating a report to a medical professional comprising the prognosis of the subject to inform a decision on treatment. In some embodiments, subjects having subtype 2 or subtype 4 RS have better prognosis (e.g., longer lifespan, fewer symptoms) than subjects having subtype 1, subtype 3 or subtype 5 RS.

In some embodiments, the detecting is achieved by comparing the sequence data of the subject with a reference genome sequence. In some embodiments, the generating a report is achieved by updating a graphical user interface. In some embodiments, the graphical user interface is a monitor.

In some embodiments, the sequence data is from cell-free DNA (cfDNA). In some embodiments, the sequence data is generated by whole genome sequencing, e.g., next generation sequencing.

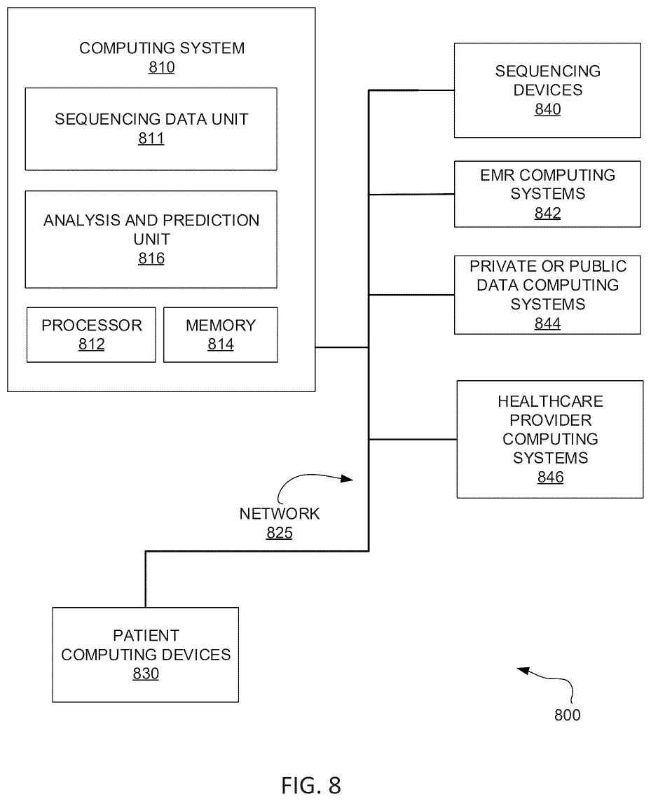

Turning attention to FIG. 8 is a block diagram of a computing environment for realizing the systems and methods for detecting health state according to embodiments of the subject matter disclosed herein. The overall computing environment 800 may be generally comprised of several sets of computing devices that are all communicatively coupled to each other through a computing network 825, such as the Internet, though the network 825 may be a local Intranet or a virtual private network or the like. These generalized categories of the coupled computing devices and/or systems include a healthcare data analysis computing system 810, one or more patient computing-devices 830, and one or more data-service computing devices, such as public or public data collection systems 844, Electronic Medical Record (EMR)/Electronic Health Record (EHR) systems 842, and healthcare provider computing systems 846. Other data-collection and/or data provision services (such as government information service computing systems, research institution computing systems, and the like) are contemplated but not shown in this figure here for brevity. Collectively, these computing devices may be used to receive and send data patient data, assessment data, diagnostic data, and/or therapeutics data. The computing system 810, includes one or more local processors 812 that utilizes one or more memories 814 in conjunction with a sequencing data unit 811 and an analysis and prediction unit 816.

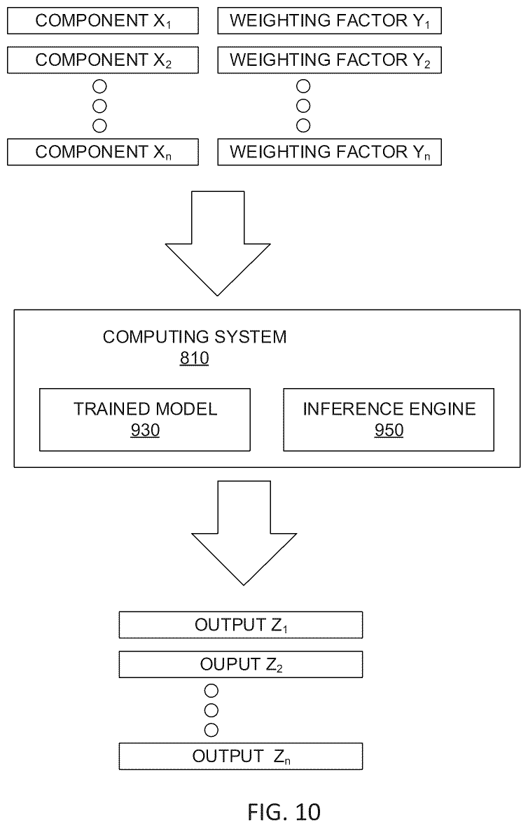

Utilizing the overall system 800 of FIG. 8, one may take advantage of sequencing and other data to inform analysis and prediction engine 816 with more accurate patient data leading to greater accuracy and predictability for further testing, diagnostics, and therapeutics. This may be accomplished by training a prediction engine as discussed further below with respect to FIG. 10.

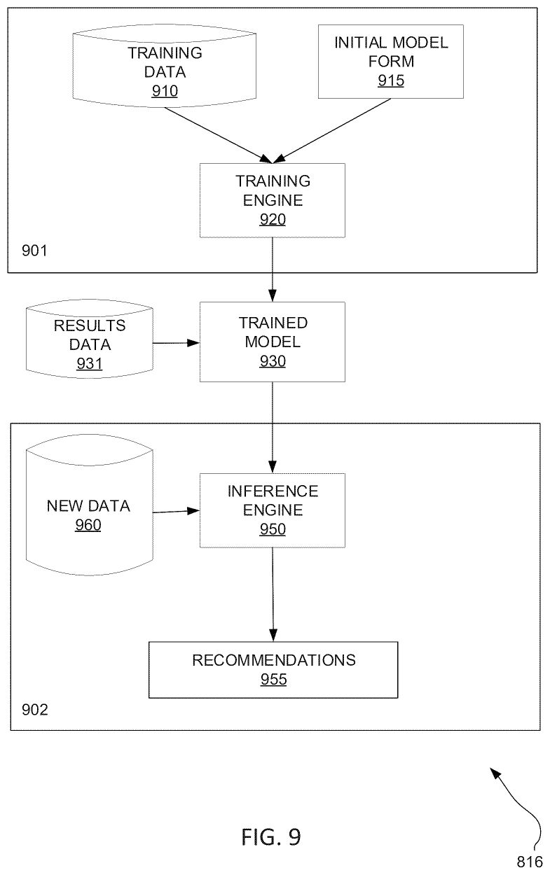

FIG. 9 is a method flow chart illustrating an exemplary computer-based method for establishing a trained prediction model and updating the trained prediction model to generate outputs according to embodiments of the subject matter disclosed herein. In this block diagram, some modules may represent functional activities, such as data collection and training, but this diagram is, nevertheless, presented in a block diagram format to convey the functional aspects of the overall analysis and prediction computing block 816 of FIG. 8. Thus, in FIG. 8, a first aggregated set of functions includes the upper half 901 of the diagram where a classifier is first established and trained for use in making predictions. Once the trained model 930 is established, the lower half 902 of the block diagram of FIG. 9 focuses on generating initial predictions to be checked against expected or historical data as well as new predictions based on new data collected.

In the upper half 901, training data 910 may be drawn from an established database of known and established sequencing data with an initial model form 915. The training data is then fed to a training engine 920 to begin establishing the trained model 930 to be used for predictions and recommendations for health care decisions. The training data may include actual collected data from historical records of a patients or may be scientific studies with best-known practices at the time of training. Further, the training data may be created based on learned judgment of best medical practices. This initial model form 915 may simply be an initial “best guess” by administrators of the analysis system. As the initial training data 910 may also include outcomes such as healthcare recommendations and predictions, a training engine 920 may begin to “train” the initial model form 915 by identifying specific data correlations and data trends that affect the effectiveness and accuracy of predictions and recommendations from the training data 910. As all relevant and/or influential correlations are determined by the training engine 920, a trained model 930 is established.

With the trained model 930 established, an inference engine 950 may then utilize the trained model 930 along with newly collected assessment data. That is, a clinician or physician may wish to use the system 800 to enhance, verify, or otherwise generate healthcare assessments and recommendations (e.g., health state, treatments, and the like) based on collected data. The system may present new data 960 in the form of collected patient data. The new data 960 may be used by the inference engine 950 that employs the trained model 930 to generate one or more assessments or recommendations 955.

The inference engine 950 may be used to generate predicted outcomes based on new data that is entered as well as based on a trained model 930 established previously from training data 910. Each of the recommendations and predictions discussed below may be influenced by one or more components as discussed above. Further, algorithms may be developed for one or more predicted outcomes based on weightings given to each of the influential inputs. In general, any set of components may have weightings that influence any predicted outcome.

FIG. 10 is a method flow chart illustrating an exemplary computer-based method for utilizing a trained prediction model 930 and delivering assessments and/or recommendations based upon a trained model according to embodiments of the subject matter disclosed herein. FIG. 10 illustrates one or more algorithms that may be realized during the establishment of the trained model 930 whereby the computing system 810 may establish specific assessments and/or recommendations (“outputs”) Z₁-Zₙ based on new data through its inference engine 950. That is, given inputs X₁-Xₙ, each with corresponding weighting factors Y₁-Yₙ, the inference engine 950 will utilize the trained model 930 to generate predicted outputs Z₁-Zₙ. Generally speaking, the weighting factors may be a result of the prediction process whereby different factors are determined to be more or less influential over the prediction processes. For example, initial weighting factors may be zero as there does not exist any predictive data yet—but as predictions emerge and comparisons to reality are determined, weightings of influential factors may also emerge. These concepts may be better understood with respect to the following non-limiting examples.

The following examples illustrate illustrative methods for illustrative compounds provided herein. These examples are not intended, nor are they to be construed, as limiting the scope of the disclosure. It will be clear that the methods can be practiced otherwise than as particularly described herein and for other compounds within the scope of the genus described herein. Numerous modifications and variations are possible in view of the teachings herein and, therefore, are within the scope of the disclosure.

### EXAMPLES

Various embodiments will be further clarified by the following examples, which are in no way intended to limit this disclosure thereto.

### Example 1: Identification of the Drivers of RS

Richter syndrome (RS) arising from chronic lymphocytic leukemia (CLL) exemplifies an aggressive malignancy that develops from an indolent neoplasm. To decipher the genetics underlying this transformation, the inventors deconvoluted admixtures of CLL and RS cells from 52 patients with RS, evaluating paired CLL-RS whole-exome sequencing data. The inventors discovered novel RS-specific somatic driver mutations (IRF2BP2, SRSF1, B2M, DNMT3, EZH2, TET2 and CCND3), recurrent copy number alterations beyond del(9p21) [CDKN2A/B], including amp(7q21.2) [CDK6] and amp(9p24) [PDL1/L2], recurrent whole genome duplication and chromothripsis, which were confirmed in a validation cohort of 45 independent RS cases and in an external set of RS whole-genomes. Through unsupervised clustering, clonally-related RS was largely distinct from diffuse large B cell lymphoma (DLBCL) but most similar to DLBCL with TP53 inactivation.

A multi-center cohort of patients with available CLL and RS samples was assembled, from which paired CLL and RS samples were collected between the years 2002 and 2020. The median time from CLL diagnosis to RS was observed over years along with a median of time from CLL sampling to RS sampling. Patients received prior CLL-directed therapies, with some patients having had exposure to novel agents, while other patients developed RS in the absence of any CLL directed therapies. Some patients had immunoglobulin heavy chain gene (IGHV)-unmutated CLL and others with IGHV-mutated disease. In total, whole-exomic sequencing (WES) was completed on the samples, of which some contained ‘duos’ (paired antecedent CLL and RS) and ‘trios’ (paired germline/normal, antecedent CLL and RS samples). Phylogenetic trees with cancer cell fraction (CCF) clustering, clonal abundance pie charts and related patient timeline in a clonally related and unrelated representative case. It was observed that some patients had multiple CLL sampling time points. The sources of CLL DNA originated predominantly from circulating mononuclear cells while RS DNA was extracted primarily from tissues preserved in formalin and embedded in paraffin (FFPE) or fresh frozen tissue DNA sources were observed.

To delineate the driver events giving rise to the RS clone, standard well-established whole-exome sequencing (WES) analysis tools were employed for the identification of somatic single nucleotide variants (sSNVs) (i.e. MuTect), indels and somatic copy number alterations (sCNAs), and for assigning a probabilistic cancer cell fraction (CCF) for each somatic event (ABSOLUTE). Additionally, 3 key steps were introduced: (i) deTiN, a tool for the recovery of somatic mutations even when the-germline control contains slight evidence of the mutation due to tumor DNA contamination (which is common in blood malignancies), thus increasing the sensitivity of detection; (ii) an optimized tool for detection of sCNAs, developed to address noisy copy number estimates introduced in FFPE specimens, which incorporates estimates of the correlation structure of coverage along the genome within a Hidden Markov Model (HMM) to infer copy number profiles; and (iii) PhylogicNDT to establish the clonal composition within each set of patient samples, by inferring the phylogenetic tree, with clones represented as nodes in the tree and branches representing the mutations acquired from a parent to child clone, thus allowing identification of RS clones within admixed samples.

Application of these tools facilitated clear identification of the CLL and RS clonal structure and relationships and enabled the identification of RS specific events. Within the CLL compartment, phylogenetic trees identified the ancestral clone (CLL^{ANC}), intermediate clones (CLL^{INT}) that arose from CLL^{ANC} and expanded upon transition to RS, as well as divergent clones (CLL^{DIV}). RS clones related clonally to CLL (i.e., sharing at least one common ancestral clone with CLL) were defined as new clones arising in the RS sample, not present in antecedent CLL, and were distinct on the basis of sSNVs and sCNAs. Additionally, based on PhylogicNDT analysis of WES data, instances of clonal unrelatedness to antecedent CLL were also identified (i.e., lacking a shared clonal history with antecedent CLL), which had been previously occasionally identified on the basis of IGHV sequencing and associated with better survival than clonally related cases. The majority of examined cases had RS clonally related to the antecedent CLL (n=45, 87%). Evolutionary relationships as determined by WES analysis (determined by comparing the immunoglobulin genes sequence inferred from WES data from both CLL and RS samples by using the MiXCR and IgCaller algorithms) were highly concordant with inferred IGHV sequence and superior to the limited available traditional IGHV sequencing, likely due to complications of sample admixture. These analyses were in line with sSNVs/sCNVs-based phylogenies except for 3 cases likely due to complications of sample admixture and limits of clonotype reconstruction from WES data. The percentage of the VH gene identity to the germline sequence assessed by IgCaller was highly concordant with conventional PCR-based IGHV mutational status (46 of 48 cases). Only 2 of 52 cases were observed to have the stereotype 8 BCR previously identified with RS risk.

The vast majority of patients examined in this cohort had RS clonally related to the antecedent CLL, unmutated IGHV, and multiple prior therapies. However, some patients had no prior therapies whereas others had exposure to novel targeted agents.

### Example 2: Identification of the RS Genomic Landscape

The genomic spectrum of RS cells was identified, acknowledging that RS harbors both mutations and CNVs specific to the RS clone as well as history of clonal mutations arising in related preceding CLL^{ANT} and CLL^{INT} clonal branches. Mutational load was observed for CLL vs RS and mutational signatures from the CLL and RS branches of the evolutionary tree were also observed. All the alterations detected in the RS cells (i.e. in the RS sample after correcting for contamination by chronic lymphocytic leukemia (CLL) cells. Samples were annotated for IGHV status, exposure to therapy before sampling and clonal relationship.

Most samples had TP53 disruption, either through TP53 mutation or 17p deletion as previously reported. In addition to known NOTCH1 mutations in RS cases, Notch pathway was also commonly affected, either by 3′UTR mutation previously described in CLL; or by FBXW7 and SPEN mutations. By MutSigCV2, the novel candidate RS driver mutations included IRF2BP2, DNMT3A, SRSF1, and EZH2. Epigenetic modifiers not typically altered in CLL were also observed upon RS transition (e.g., CREBBP, EP300, KMT2D).

Strikingly, numerous copy number events were observed in RS compared to CLL. By GISTIC2.0, the most common recurrent amplifications were focal amplifications of and arm level events. Among the arm level events was loss of chromosome 9p [CDKN2A/B]), which was observed in some patients. Notably, a few cases were uncovered with whole-genome doubling, which was observed more commonly in RS deriving from CLL patients with mutated IGHV genes (M-CLL). Trisomy 12 was associated with Notch alterations, as previously reported.

The instant analytic approach was implemented to assess the full spectrum of somatic alterations within the RS history, comprising CLL^{ANC} and CLL^{INT} clonal branches and the RS clone, and separate from the divergent CLL; and strictly present in RS cells through computational isolation of the RS lineage. To uncover drivers of transformation, MutSigCV2 (q<0.1) was applied to both the clones representing RS history and RS clones. Additionally, to enhance power to detect known drivers, restricted hypothesis testing was used using established curated lists of CLL and DLBCL drivers.

In addition to known CLL drivers, several new candidate RS drivers were identified, and by subsequently focusing the MutSigCV2 analysis to evaluate events restricted to the RS clones, it was demonstrated that these drivers were indeed first detected at the transition to RS. These included missense and nonsense/frameshift mutations across 7 patients in Interferon Regulatory Factor 2 Binding Protein 2 (IRF2BP2), which encodes an IRF2-dependent transcriptional corepressor, previously identified as mutated in subsets of patients with diffuse large B cell lymphoma and primary mediastinal B cell lymphoma (PMBCL) and the N1 subtype of DLBCL. Alterations in the gene encoding the DNA methyltransferase enzyme DNMT3A, all predominantly inactivating mutations, were detected in 8% of patients; recent genetically engineered murine models to model this alteration have confirmed the CLL-driving function of this alteration and impact on NOTCH signaling. B2M loss through inactivating mutations, a known mechanism of immune escape in hematologic and solid tumors, was observed in 3 patients. The serine/arginine-rich splicing factor SRSF1 was mutated in 4 cases and highlights the potential importance of mRNA splicing in RS in addition to its known role in CLL. Of note, cases with mut-SRSF1 did not overlap with those bearing antecedent CLL SF3B1 alterations. Furthermore, SRSF1 is known to interact with MYC in lymphoma cells. Additional significantly recurrent sSNV events in RS also included PIM1, a likely target of somatic hypermutation (SHM) and TP53.

Strikingly, in contrast to CLL, the inventors observed numerous and frequent copy number events in RS, including del(17p) (TP53, 63%) and del(9p21.3) (CDKN2A/B, 19%). Arm-level loss of 9p (encompassing CDKN2A) was seen in an additional 5 patients. Recurrent focal events, aside from known CLL drivers, included del(15q13.11) (encompassing the MGA locus, 21%), amplification (amp) of chromosome 8q24 (MYC, 15%), del(7q36) (EZH2, POT1, 11.5%), and amp(13q31.2) (ERCC5, miR-17-92 12%), the majority of which were detected in the RS phase or CLL^{INT}. Novel changes enriched in RS clones included amp(9p24) (PDL1/L2, 8%), del(16q12) (11.5%), del(18q22) (BCL2, 8%), and amp(7q21.2) (CDK6, 11.5%). Examination of significant events restricted to the RS clones identified further changes on chr1 (del(1p), amp(1q23) (MCLI, NOTCH2, BCL9, PDE4DIP)).

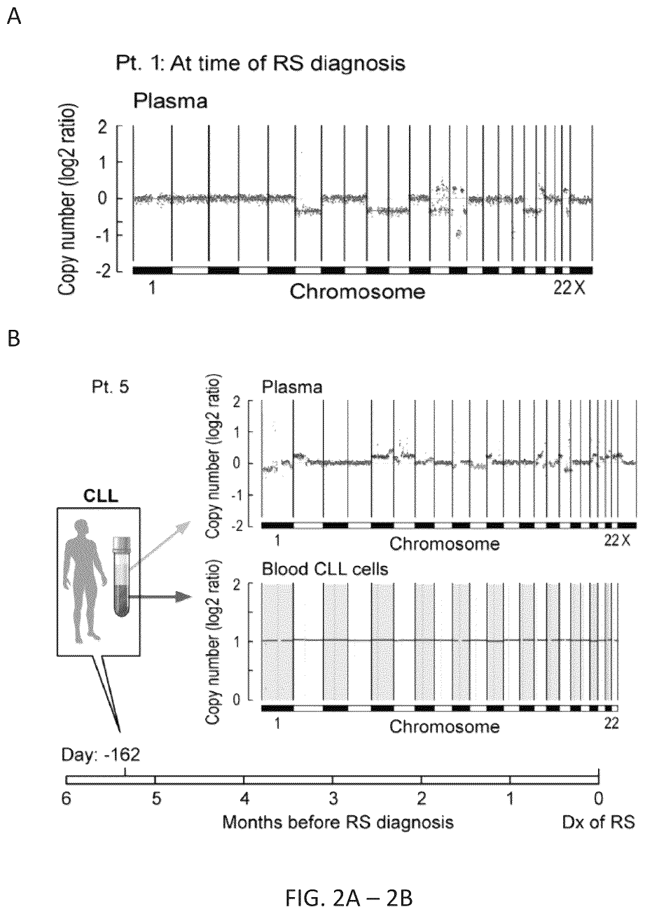

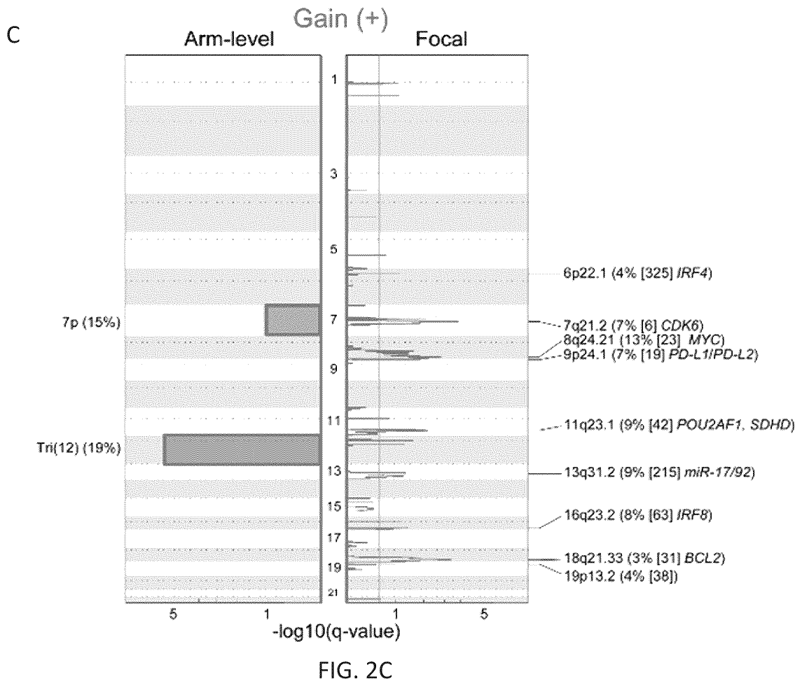

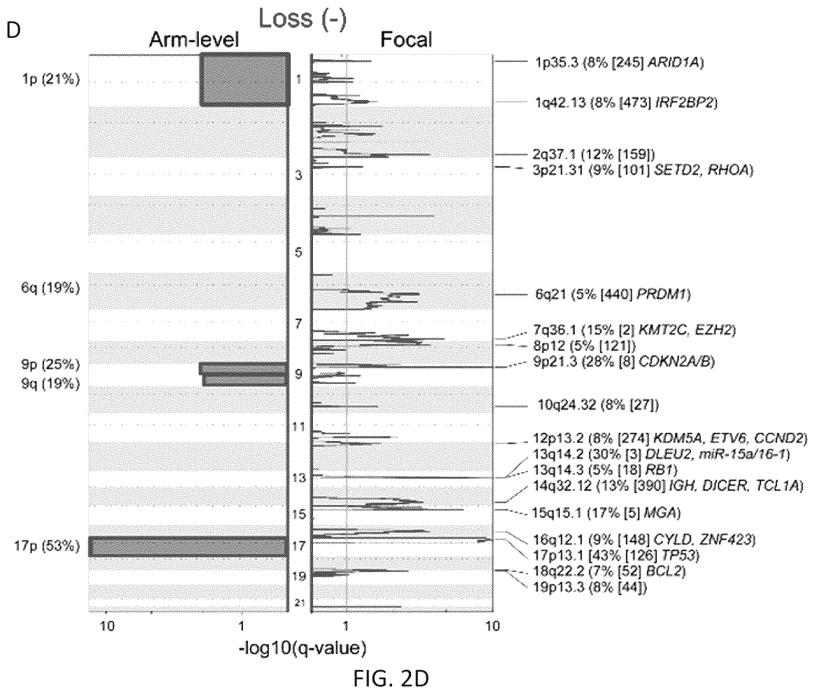

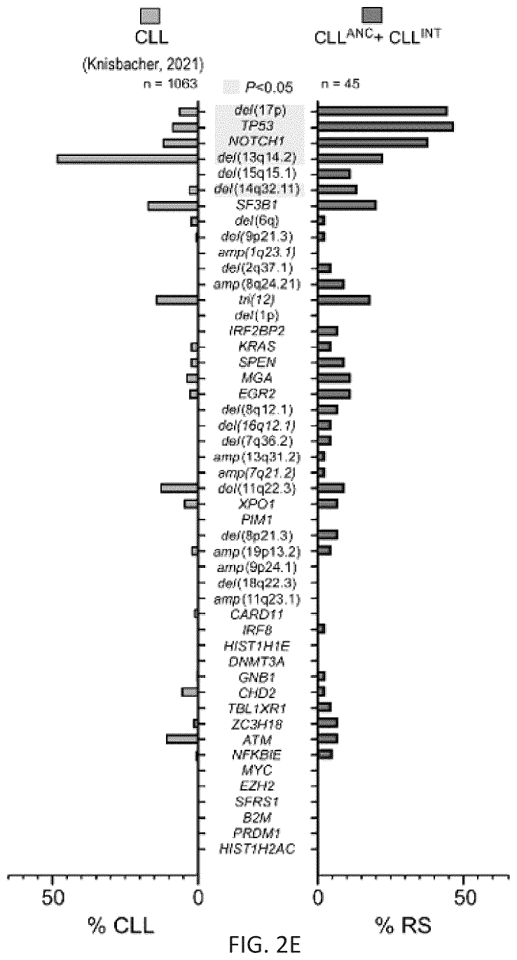

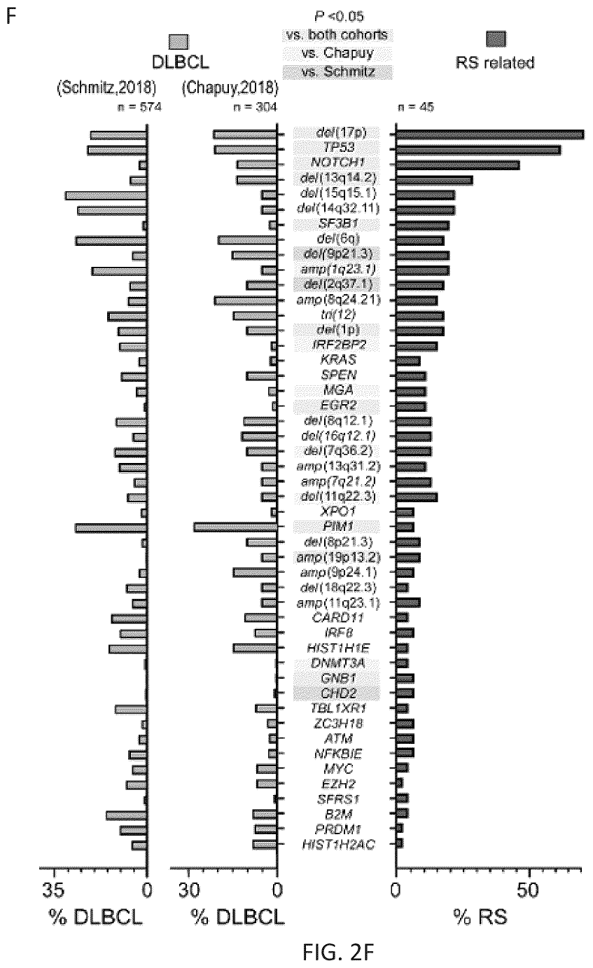

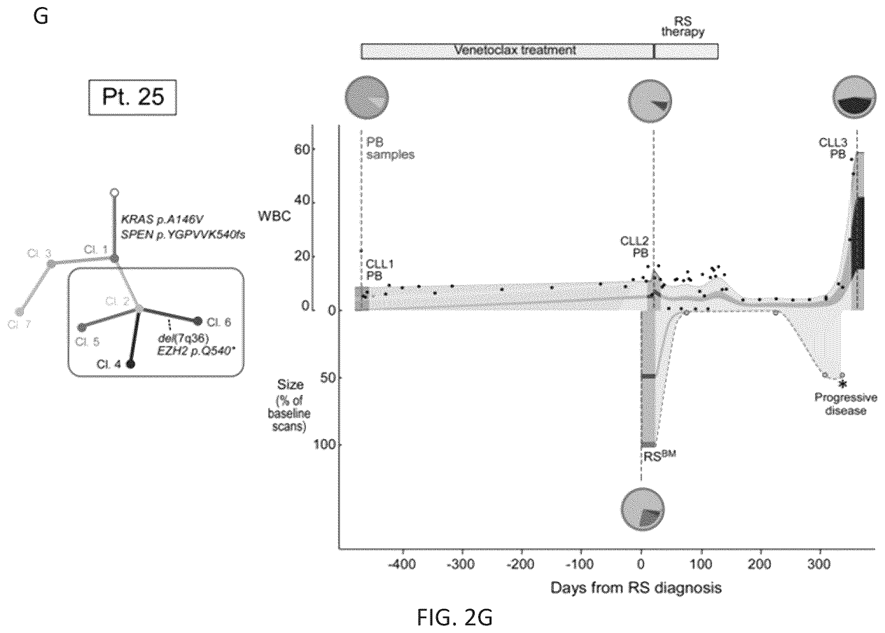

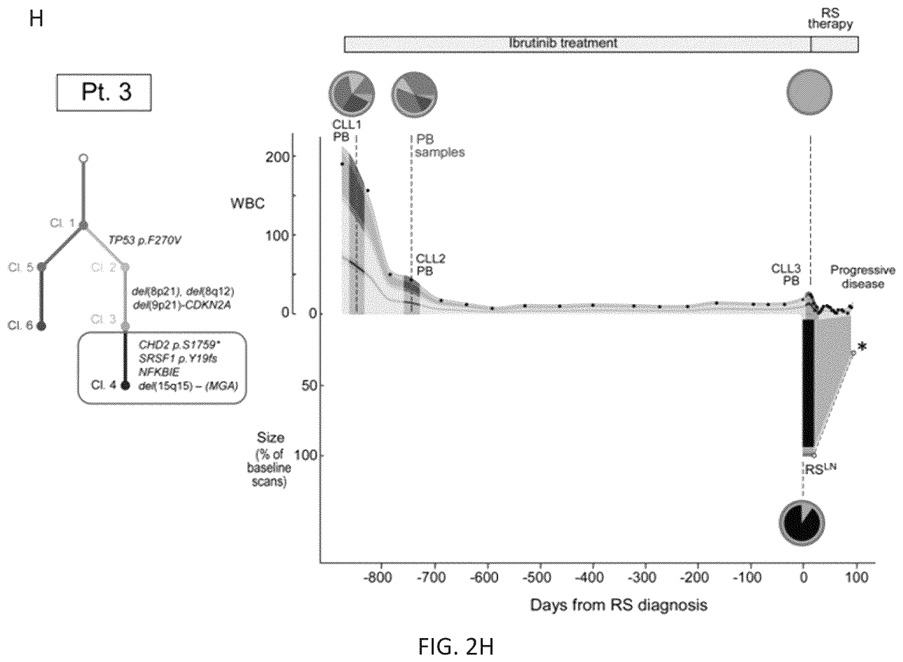

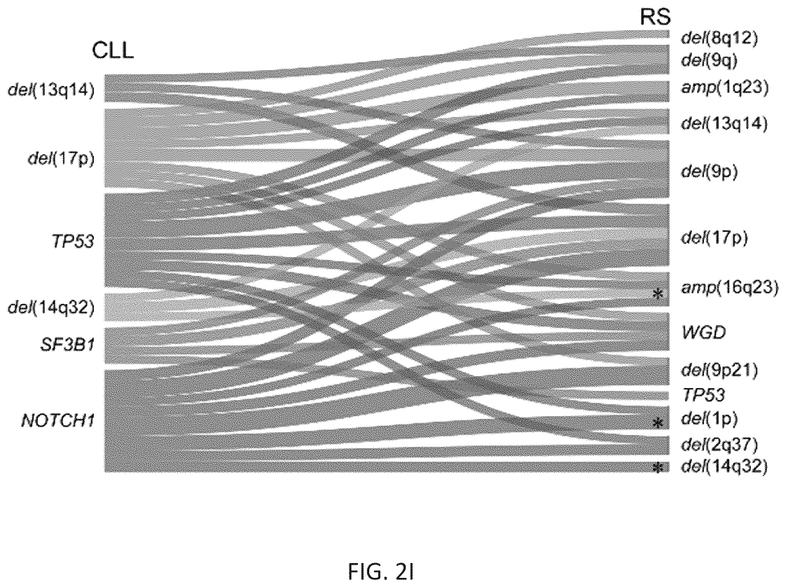

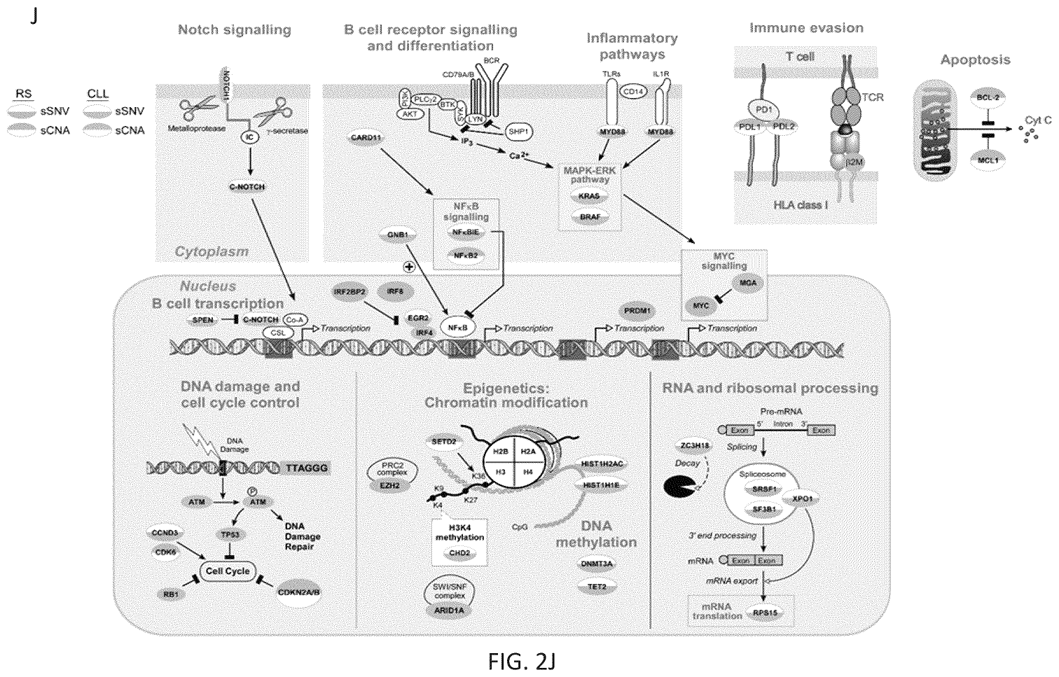

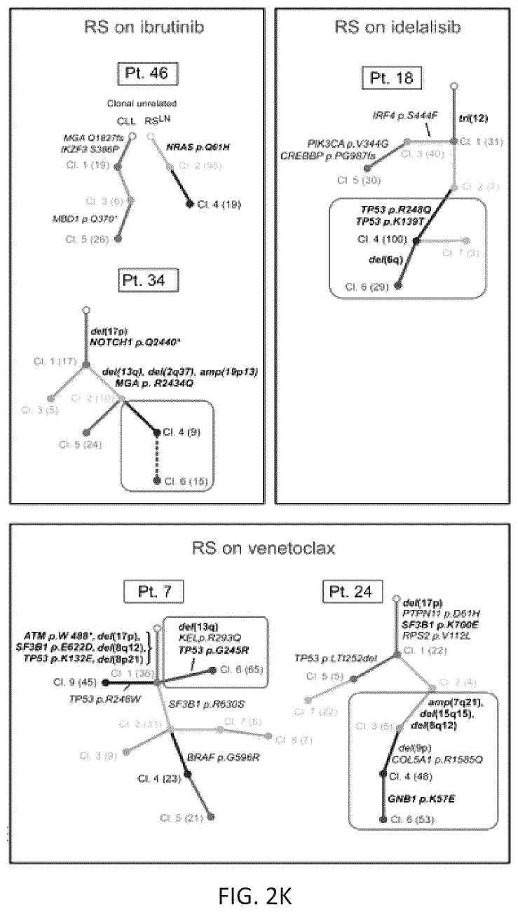

Through comparison with large-scale analyses of CLL, lesions that appeared to predispose for RS were observed, given their relative enrichment in CLL^{ANC} These sSNVs and sCNAs included mutated TP53, NOTCH1, and del(17p), del(14q32) (all p<0.05, exact binomial test; FIG. 2E) but not tri12, mut-SF3B1, or del(11q). While del(13q) was not enriched in CLL^{ANC} and was higher in CLL (p<0.05), del(13q) in the RS cohort encompassed RBI loss in 11 of 13 patients. While not meeting significance by MutsigCV2, mutational loss of RBI was further seen in 2 of these patients, thereby generating biallelic loss. The instant analysis implicates several events in the process of transformation that occur predominantly in the RS phase (FIG. 2E). For example, CDKN2A loss and del(7q36) were most frequently observed as new RS events and del(1q) and amp(1q23) were only seen in RS. By contrast, tri12 only was in CLL^{ANC}. Compared to known DLBCL drivers, several RS-clone specific lesions are also observed in DLBCL although many were enriched in RS compared to DLBCL (mut-TP53, -NOTCH1, -IRF2BP2, -MGA, and del(17p)) (all p<0.05, exact binomial test); FIG. 2F).

The candidate RS driver mutations included missense and nonsense/frameshift mutations across 7 patients in Interferon Regulatory Factor 2 Binding Protein 2 (IRF2BP2), which encodes an IRF2-dependent transcriptional corepressor, previously identified as mutated in the N1 (NOTCH1) subtype of DLBCL and primary mediastinal B cell lymphoma. Inactivating mutations in DNMT3A, a gene encoding a DNA methyltransferase, were detected in 8% of patients and observed in one RS patient previously, recent genetically engineered mice to model this alteration have confirmed its CLL-driving function and impact on NOTCH signaling. B2M loss through inactivating mutations, a mechanism of immune escape in hematologic and solid tumors was observed in 3 patients. The serine/arginine-rich splicing factor SRSF1 was mutated in 4 cases and highlights the likely importance of mRNA splicing in RS in addition to its known role in CLL. Of note, cases with mutated SRSF1 did not overlap with those bearing antecedent CLL SF3B1 alterations, consistent with mutual exclusivity of splicing factor mutations across cancers. Furthermore, SRSF1 is known to interact with MYC in lymphoma cells. EZH2 hotspot alterations were observed in two clonally unrelated RS cases, as in DLBCL^{18,20}, while EZH2 frameshift was seen in one clonally related RS case. Additional significantly recurrent sSNVs in RS shared overlap with DLBCL and included MYC, IRF8, and PIM1.

Strikingly, numerous somatic copy number alterations (sCNAs) were observed in the RS discovery cohort, including del(17p) [TP53, 63%] and del(9p21.3) [CDKN2A/B, 19%], with arm-level loss of 9p in an additional 5 patients. Recurrent focal events, beyond common CLL drivers, included del(15q13.11) [MGA and B2M, 21%], amplification (amp) of chromosome 8q24 [MYC, 15%], del(7q36) [EZH2, POT1, KMT2C 11.5%], and amp(13q31.2) [ERCC5, miR-17-92 12%], which have been observed in high-risk CLL³². Changes not previously reported in CLL or RS included amp(9p24) [PDL1/L2, 8%], del(16q12) (11.5%), del(18q22) (8%), and amp(7q21.2) [6 genes, including CDK6, 11.5%], del(1p), amp(11q) [POU2AF1, SDHD] and amp(1q23). Notably, whole genome doubling (WGD) was seen in 15% of cases.

These major findings of recurrent RS-specific gene mutations and sCNAs and WGD were confirmed in the validation cohort (n=45) (FIGS. 6A-6X, bottom circles) and 14 previously characterized RS genomes (FIGS. 6A-6X, bottom squares). Moreover, combined analysis of our discovery and validation cohorts together provided additional power to further detect novel RS drivers, with mutations in CCND3, TET2 and BRAF emerging as significant across the 97 patients (FIGS. 6V-6X). Furthermore, additional focal sCNAs were detected: amp(18q21.33) [BCL2], amp(16.23.2) [IRF8], amp(6p22.1) [IRF4], del(19p13.3), del(12p13.2) [KDM5A, ETV6, CCND2], del(1q42.13) [IRF2BP2], del(10q24.32), del(1p35.3) [ARID1A], del(3p21.31) [SETD2], and del(13q14.2) [RBI] in addition to arm-level events (FIGS. 2C-2D).

Through comparison of the 45 clonally related cases in our discovery cohort with prior large-scale analyses of CLL, lesions that appeared to predispose for RS were identified, given their relative enrichment in CLL^{ANC+INT}. These sSNVs and sCNAs included mutated TP53 and NOTCH1, del(17p) and del(14q32) but not tri(12), SF3B1, or del(11q) (all Q<0.05, exact binomial test). The distribution of drivers in the 45 clonally related RS was enriched in TP53, del(17p), NOTCH1, del(13q14.2), del(1p), amp(19p13.2), SF3B1, EGR2, and GNB1 (all Q<0.05, exact binomial test). IRF2BP2, MGA and DNMT3A frequency was higher in RS compared to a cohort of 304 de novo DLBCLs.

Specific mechanisms underlying the transformation to RS were identified through in-depth parallel examinations of the CLL (peripheral blood) and the RS compartments over time in patients from whom multiple serial samples were obtained during novel agent CLL therapy in the years prior to RS. Pt 25 first achieved response to venetoclax and secondarily progressed after 13.5 months with subsequent biopsy-confirmed RS in lymph node (LN) and bone marrow (BM) (FIG. 2G). Strikingly, the RS specimen did not harbor any known lesions associated to venetoclax resistance in CLL but instead carried both an inactivating EZH2 frameshift mutation and del(7q36), which encompasses the EZH2 locus, and hence homozygous abrogation of EZH2 activity. This clone was additionally detectable in the blood a few weeks after RS diagnosis and again following VR-EPOCH, with varied subclonal composition. Inactivating EZH2 mutations have been demonstrated to be oncogenic in other malignancies, including T-ALL, where it co-occurs with NOTCH alterations. This case illuminates the role of epigenetic remodeling as a mechanism of transformation, here generated by EZH2 loss, with few other genetic changes detected between the CLL and RS phases. In another example, Pt 3 was originally detected to have TP53 mutation during CLL phase, and developed nodal RS while on ibrutinib therapy. This patient was observed to have a distinct RS clone emerging from an expanding aggressive CLL subclone (clone 3) in the peripheral blood that was marked by additional CN loss, including the CDKN2A/B locus (FIG. 2H). Newly acquired genetic changes in the RS clone included a nonsense mutation in the chromatin modifier CHD2, a frameshift mutation in SRSF1, a splice-site mutation in NFKBIE and del(15q15) (MGA loss). This case illustrates successive CN alterations in a TP53-mutated CLL in the progression towards RS and again highlights epigenetic modification, this time in cooperation with splicing alterations and MYC signaling (through MGA loss), as a key altered cellular process in the transformation to RS.

Therapies targeting BTK (e.g. ibrutinib), BCL2 (venetoclax) or PI3K-delta (e.g. idelalisib) have revolutionized CLL therapy, and yet failed to prevent transformation. RS is a recognized mechanism of therapeutic resistance, which prompted the inventors to study the 15 patients of the cohort presenting transformation to RS while on targeted therapies (8 on ibrutinib, 4 on venetoclax and 3 on idelalisib). None of these RS cases carried typical resistance mutations to targeted agents (BTK, BCL2), while one ibrutinib-exposed patient had del(8p) (Pt 3) and one venetoclax-exposed patient had amp(1q) (Pt 24), previously described sCNA drivers of CLL therapeutic resistance. These findings suggest that transformation to RS is primarily a process distinct from acquired targeted agent resistance in CLL. Consistent with the entire cohort, these patients displayed diverse clonal evolutionary paths to RS, with acquisition of genetic alterations in the previously noted pathways of epigenetics, MYC signaling, DNA damage, splicing and cell cycle dysregulation.

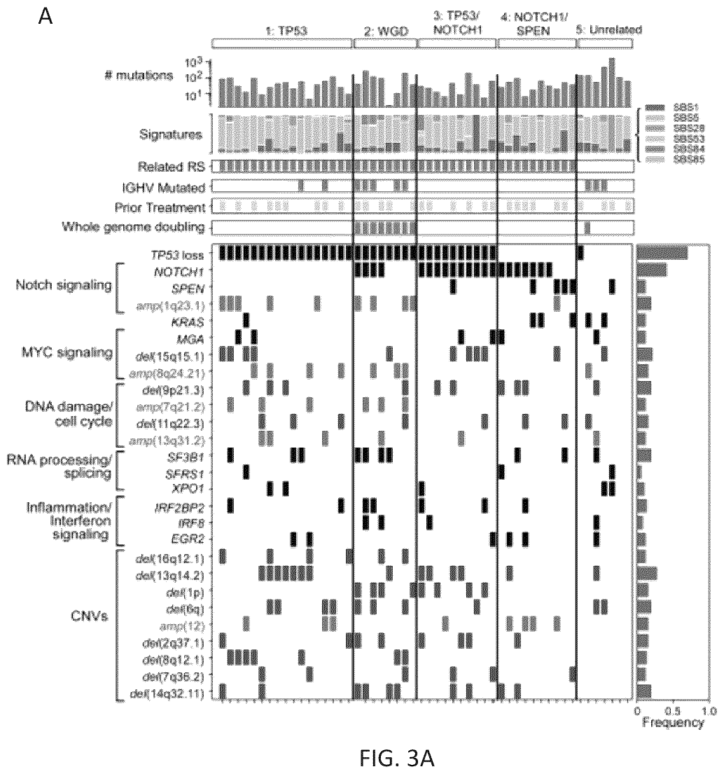

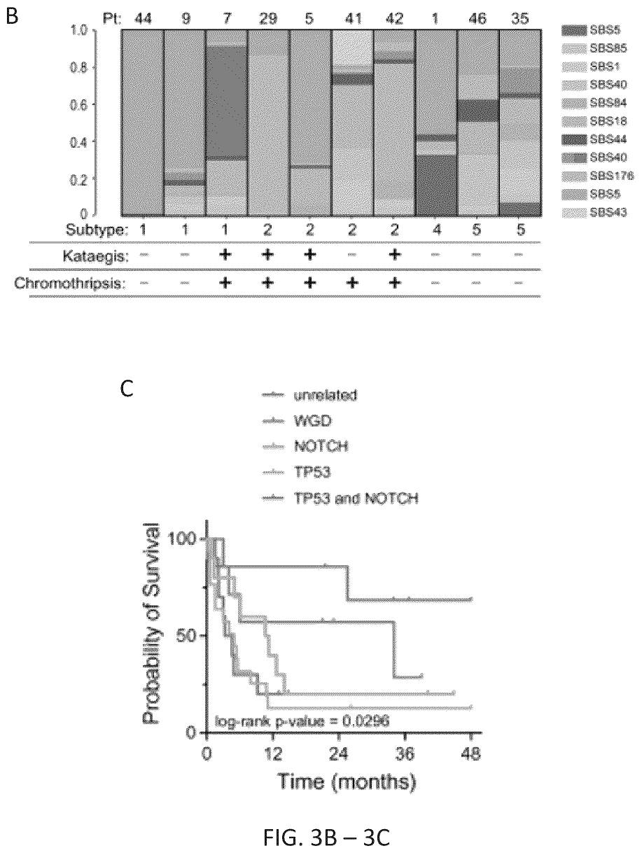

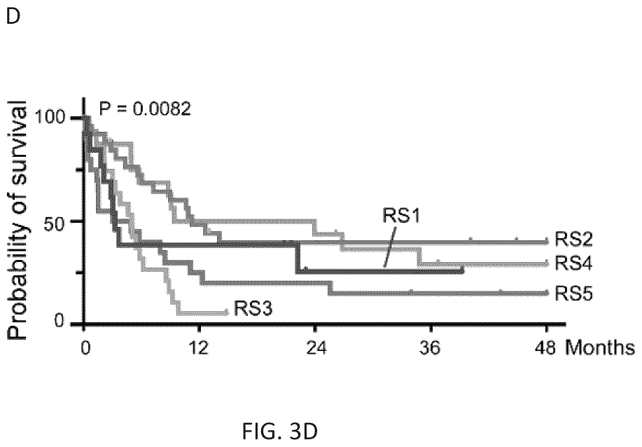

As discussed above, two of these patients had serial samples procured in the years prior to RS, enabling in-depth clone tracking to provide insights into the development of transformation (FIG. 2G-2H). Pt 26 achieved response to venetoclax and secondarily progressed after 13.5 months with biopsy-confirmed RS in lymph node (LN) and bone marrow (FIG. 2G). Strikingly, the RS specimen carried both an inactivating EZH2 frameshift mutation and del(7q36), and hence homozygous abrogation of EZH2 activity, highlighting the impact of epigenetic remodeling on transformation. This clone was detectable in the blood a few weeks after RS diagnosis and again at progression after treatment (FIG. 3c, second line from the top). In another example, Pt 3 developed nodal RS that evolved from TP53-mutated CLL while on ibrutinib therapy The RS clone emerged from an aggressive CLL subclone (clone 3) in the peripheral blood, marked by focal loss of the CDKN2A/B locus, that appeared to be selected by ibrutinib therapy (FIG. 2H). Newly acquired genetic changes in the RS clone included a nonsense mutation in the chromatin modifier CHD2, a frameshift mutation in SRSF1, a splice-site mutation in NFKBIE, and del(15q15) [MGA loss]. Thus, successive acquisition of sCNAs in a TP53-mutated CDKN2A/B deleted CLL as well as epigenetic modification, altered splicing and MYC signaling (through MGA loss), are demonstrated to be key altered cellular processes in the transformation to RS.

The inventors then evaluated the relative timing of each putative driver event in 58 related RS cases from the combined discovery and validation cohorts, for which paired samples enabled clonal deconvolution. ATM mutations, tri(12) or SF3B1 mutations were already present in CLL^{ANC} and TP53 alterations (mutations and/or del(17p)) and NOTCH1 mutations as well as del(15q15.1) [MGA] were predominantly CLL events (P<0.05, Q<0.2, McNemar test). In contrast, del(9p21), del(9p), del(9q), del(2q37), amp(1923), and del(6q) were most frequently observed as new RS events (P<0.05, McNemar test, Q<0.2), and the detection of WGD was restricted to the RS clones. By systematically evaluating instances of co-occurrence of a CLL driver with a RS driver (since these pairs would reflect the acquisition of a transforming lesion atop of a pre-existing and potentially RS-priming one), the inventors identified preferred genomic trajectories driving transformation (FIG. 2I). Per CLL driver, we calculated the probability for acquiring any of the RS drivers using a network analysis. Trajectories from CLL to RS reaching significance (P<0.05; Q<0.4) included NOTCH1 to del(1p), NOTCH1 to del(14q32) and del(14q32) to amp(16q23).

Overall, these findings highlighted alterations of NOTCH1, DNA damage response and MAPK pathway as preexisting in CLL. Further, the analysis highlighted epigenetic changes, interferon/inflammatory signaling, cell cycle deregulation and immune evasion-whether by sSNVs or by sCNAs—as the major mechanisms newly occurring at transformation (FIG. 2J).

### Example 3: RS Specific Alterations and Mechanisms of Transformation

While many of the high prevalence genomic alterations of RS were also CLL drivers, the ability to distinguish between CLL and RS clones allowed pinpointing of those genetic alterations that were acquired at the Richter phase, marked the transition of CLL to RS and thus could be used to better understand the transformation process and molecularly define RS.

Novel RS-specific alterations included EZH2 mutations, which are well documented in other lymphomas (hotspot, 641) and myeloid malignancies (FS inactivation) yet absent from prior large scale CLL WES analysis. EZH2-activating hotspot mutations were observed in clonally unrelated RS, while inactivating mutation (FS) and frequent focal deletions were observed across clonal related RS. DNMT3A inactivation were observed in several new RS cases. IRF2BP2 (Interferon regulatory factor 2 binding protein 2) encodes an IRF2-dependent transcriptional corepressor and was mutated in 8 patients (3 sSNVs were in the affect the same S195 residue). IRF2BP2 mutation was recently described in subsets of patients with diffuse large B cell lymphoma. SRSF1 (serine/arginine-rich splicing factor) was mutated in some cases and underlines the potential importance of mRNA splicing in RS in addition to its known role in CLL. SRSF1 has been shown to interact with MYC in lymphoma cells.

The transformation to RS is highlighted by in depth examination of both the CLL compartment (peripheral blood) over time as well as at the time of RS sampling, as highlighted (FISH). Pt A had excellent CLL control with minimal disease on venetoclax when progressive lymphadenopathy was observed with biopsy showing RS. The RS strikingly had both EZH2 frameshift inactivation. This clone was subsequently detectable in the blood at relapse and three separate RS subclones were detected at different sample times. Pt B developed nodal RS transformation with distinct RS clone emerging from a CLL clone 3 in the peripheral blood that was first detectable at the time of RS diagnosis.

### Example 4: Timing Modeling

Through application of a league model, timing of genetic events could be inferred across the cohort. Indeed in the progression of CLL to RS, a few were early events while others were later. Supporting this, drivers based on clonal and subclonal composition in both RS and CLL were dissected and striking patterns of clonal expansion (CLL^{INT}) were seen in some drivers upon RS transition, highlighting biological pathways that contribute to the development of aggressive lymphoma-TP53, MGA, CHD2.

### Example 5: RS Subtypes and Common Disease Trajectories

From initial analysis, M-CLL cases were observed to be distinct genetically, with high rates of WGD and a higher percentage of clonal unrelated cases. WGD was rarely seen or not seen in UM-CLL RS. This suggests different biologically processes governing transformation despite shared TP53 aberration as an early driver in both sets of patients. Within the transformed UM-CLL, two distinct subtypes were observed, defined by TP53 aberrant disease with frequent CNVs.

Molecular profile of unrelated cases were marked. TP53 intact disease was marked by T12 and frequently enriched in NOTCH1.

### Example 6: Mutational Signatures and Patterns in RS

A hallmark of RS clones was the higher mutational rates compared to the CLL clones from which they developed (2.47 vs. 0.86 Mut/Mb, p<0.0001) prompting exploration of the mutational signatures. AID signature was found in a few cases which is in line with IGLL5 or PIM1 mutations. Whole-genome sequencing (WGS) data was performed in some cases and good correlation was found with the related WES data.

In two of the clonal related cases, two independent CLLs were observed, each with a unique IGHV sequence (RIC008, RIC006) and clonal and subclonal structure, with only one giving rise to RS and another marking a clonal unrelated CLL case.

To assess feasibility of non-invasive detection of RS events, a total of 16 patient plasma samples from 9 RS patients (CLL phase, time of RS, (within 6 months leading up to diagnosis using ultra-low pass whole genome sequencing were examined. Indeed, it was found that RS specific changes could readily be identified in the peripheral blood with a few days before RS diagnosis. This process first creates a high-quality library from cfDNA, using universal molecular identifiers (UMIs) to tag each molecule, and then using ˜1-5% of the library to perform ULP-WGS at ˜0.1-0.5× coverage. Initially ichorCNA, a method specifically designed to analyze cfDNA, that can identify large-scale (>1 Mb) gains/losses in the genome to assess the fraction of tumor DNA in the cfDNA79, was developed. More recently, a new probabilistic method (currently called tufEST) was implemented that combines both CNA and fragment length aberration to infer cfDNA tumor fraction. Compared with ichorCNA, the tufEST method has higher accuracy based on manual review of the data output as well as higher sensitivity to detect extremely low tumor fractions (˜0.3%) from cfDNA.

### Example 7: Dynamics of Transformation in Single-Cell Resolution

To further determine the processes involved in the genetic instability features identified in RS, bulk RNA-seq analysis was performed on high-purity paired RS and CLL samples from 7 individuals. Comparison of the RS to CLL transcriptomes identified significantly upregulated transcripts in RS and in CLL (log₂fold change >1, qval<0.05).

To examine the expression changes and clonal structure of CLL transforming to RS at high-resolution, single-cell RNA-sequencing of RS biopsy specimens obtained at the time of RS diagnosis for 5 patients was performed. After flow cytometric sorting of cells by size to include representative populations of both CLL and RS, scRNA-seq was performed using 10× genomics platform followed by clustering using Seurat V3. To comprehensively map the genetic changes of RS and CLL clones to transcriptional clusters, the same novel HMM-based approach as described for the bulk WES analysis was adapted based on segmentation of SNP-het sites to clearly identify the sCNA alterations in the single-cell RNA data. This method greatly improved the signal to noise ratio over other commonly used methods (inferCNV) and, given the frequent defining sCNAs of RS, allowed the identification of RS and CLL clones, which mapped to distinct transcriptionally-identified cell populations. Interestingly, RS clones displayed a much higher UMI/cell and genes/cell compared to CLL, as has been seen in other aggressive hematologic malignancies.

### Example 8: RS is Defined by Distinct Molecular Subtypes

Evaluation of mutational profiles of samples from the 52 patients, based on combined CLL and RS WES data, revealed signatures of aging (SBS1/SBS5) and activation-induced cytidine deaminase AID (SBS84/85), in line with prior studies of CLL. A signature consistent with polymerase epsilon (POLE) mutation (SBS28) was detected (FIG. 3A), which was particularly enriched in a clonally unrelated case (Patient 30) having a deleterious POLE mutation, resulting in >2,000 somatic mutations. To more deeply assess the molecular mechanisms underlying transformation to RS, whole-genomes generated from a subset (11 trios, or 21% of the cohort) were analyzed. WGS-determined phylogenetic trees for the 9 of 11 clonally-related RS samples allowed for improved distinction of CLL vs RS clones and were highly concordant with the phylogenies already determined by WES. The RS clones of the other two cases did not share a more distant evolutionary history, consistent with clonally unrelated RS. Signature analysis of 10/11 evaluable genome trios revealed signatures associated with prior treatment (SBS17b), reactive oxygen species (SBS18), AID (SBS84 and SBS85), defective DNA mismatch repair (SBS44) and aging (SBS1/SBS5/SBS40) in RS clones. Recent large studies of CLL (P01 cite) have not demonstrated SBS44 or SBS17b, highlighting these as RS-specific mechanisms of mutation. SBS44 was also observed in recent studies and the present data demonstrates that this signature is not tissue dependent and indeed RS-specific. A higher number of such regions were identified across RS genomes in 4 of 11 cases compared to paired CLL samples (FIG. 3B). Recurrent regions of kataegis across samples included the IGH locus (chr 14) and IGLL5.

To further explore the role of each genomic alteration in the transformation process, the relationship between CLL and RS drivers was evaluated in each individual patient with related RS. Instances in which a CLL driver was found together with a RS driver within the same sample were identified, as these pairs reflect the acquisition of a transforming lesion atop of a pre-existing and potentially Richter-priming one. This provides the full-spectrum of preferential genomic trajectories driving transformation. For each CLL driver, the probability to acquire each RS driver was calculated. This network highlights NOTCH1 mutation occurring in CLL to TP53 alterations acquired at transformation as the most frequent trajectory of evolution to RS.

Integration of the WGS analysis along with WES results led the inventors to observe five broad molecular patterns. Pattern 1 was the largest (n=17, 33%), arising predominantly from CLL with unmutated IGHV genes (U-CLL) (88%), and marked by TP53 disruption, either through TP53 mutation (n=2, 12%) or del(17p) (n=2, 12%) or both (n=76, %) (FIG. 3A). This group was further marked by successive accumulation of copy number aberrations and altered MYC, either through MGA loss or MYC gain. Another hallmark of this group was frequent large copy number gain of chromosome 1p23, encompassing the MCLI, NOTCH2, BCL9 and PDE4DIP locus.

Pattern 2 RS (n=8, 15%) also contained TP53 loss but notably displayed whole genome doubling (WGD) and was constituted by most of the clonally-related M-CLL cases in the cohort. Moreover, a striking feature of this subgroup was not only the presence of increased regions of kataegis but also regions of underlying chromothripsis with numerous structural variants (SVs) across multiple chromosomes (Methods). Chromothripsis was observed to occur in regions likely contributing to RS pathogenesis, including 7q21 (CDK6) (pt 41), 11q13 (CCND1) (pt 29) and 9p24.1 (PD-L1/L2) (pt 41) with the majority of regions being patient-specific and not recurrent. The inventors' detection of WGD together with chromothripsis in this group suggests that for Pattern 2 cases, evolution was not a gradual process marked by successive genetic hits but rather a catastrophic event.

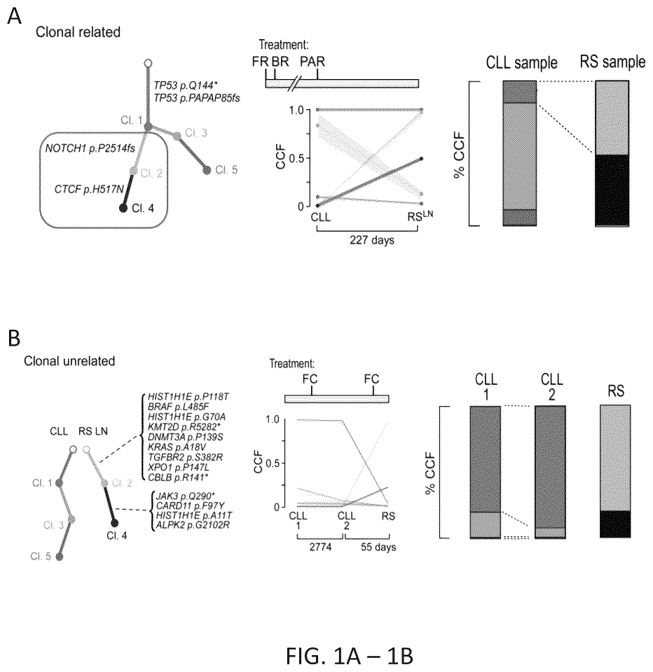

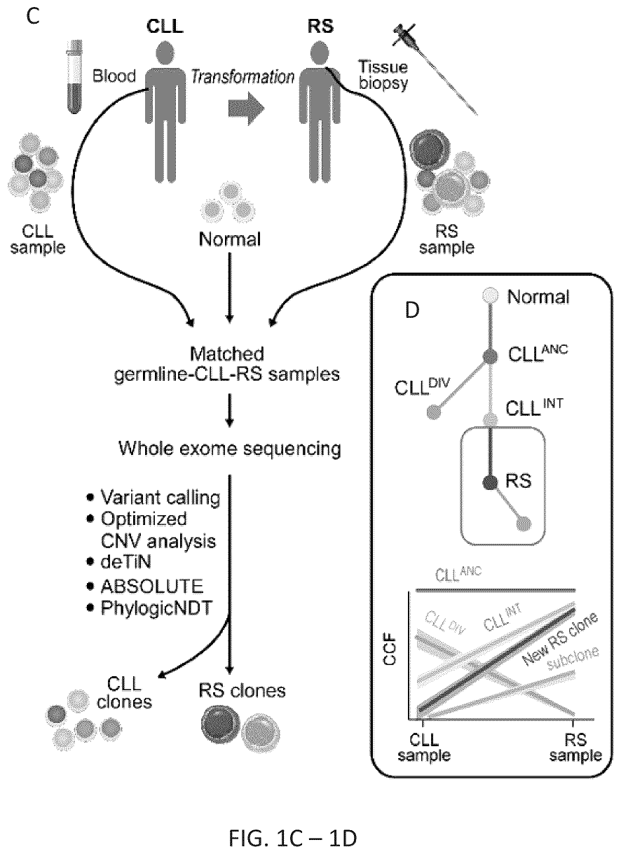

A third pattern (n=10, 19%) included patients with both TP53 alterations and NOTCH1 mutations. In patients where order of acquisition could be determined by clonal trajectories, NOTCH1 preceded TP53 aberration in 5 cases (Patients 8, 21, 22, 33, 36), while alterations in TP53 preceded NOTCH1 in 2 cases (Patients 32 and 39; FIG. 1A). This class again was marked by frequent MGA loss and numerous CNVs, and enriched for del(1p) and del(2q37).

The fourth group (n=10, 19%) also arose from U-CLL, and was notable for presence of NOTCH1 mutations (6 patients), or mutations in the NOTCH1 regulator SPEN (3 patients) or both NOTCH1/SPEN mutations (1 patient) without concurrent TP53 or aberration in chromosome 17p. This subset displayed fewer CNAs, including fewer MGA and MYC alterations, absence of del(13q), and enrichment in tri(12). Of note, Pattern 4 was associated with a higher rate of mutation in KRAS, further supporting a distinct evolutionary trajectory for this group.

The final pattern consisted of clonally unrelated cases (n=7, 13.5%), which appeared to harbor a higher frequency of genetic drivers previously reported in de novo Diffuse large B-cell lymphoma (DLBCL) and Follicular lymphoma (FL), such as hotspot mutations in EZH2. Each example of clonally unrelated RS appeared to have a unique set of mutations, without seeming to fit into any particular DLBCL molecular subtype. Notably, these cases were enriched in M-CLL and previously untreated CLL, and for the most part, lacked both TP53 and NOTCH1 alterations.

The four clonally related patterns did not associate with prior CLL treatment history. While only a subset of the instant cohort included patients on targeted novel agent therapy (n=13), these individuals did not associate with any specific RS pattern. Similarly, RS with no prior CLL treatment exposure was observed across all patterns. Median OS differed across the patterns: median OS was not reached for clonally unrelated cases (Pattern 5), was 34 months for cases with WGD (Pattern 2), 11 months for NOTCH (Pattern 4), 5 months for TP53 (Pattern 1) and 4 months for TP53-NOTCH group cases (Pattern 3) (log-rank P=0.03) (FIG. 3C).

To assess the degree of similarity between RS and DLBCL, the inventors performed unsupervised non-negative matrix factorization (NMF) clustering on our 97 RS cases along with 304 previously characterized DLBCL samples based on the identified RS genetic alterations together with known DLBCL drivers. The majority of RS (75 of 97 RS cases) clustered together, largely separately from DLBCL. The DLBCL cases closest to RS comprised DLBCL C2, previously characterized as consisting of biallelic TP53 inactivation, frequent CDKN2A/B loss and del(13q14) (Rb1). Of note, 7 of 8 clonally unrelated RS clustered with DLBCL (Fisher's exact test P=6.75×10⁻⁶), with membership across the DLBCL molecular clusters 1, 3, 4 and 5, highlighting unrelated RS as a diverse entity with molecular features similar to de novo DLBCL, consistent with independent cancers.

NMF consensus clustering was further employed to analyze the WES sequencing data across our RS validation and discovery cohorts, thus defining 5 RS molecular subtypes. Three subtypes (subtypes 1, 3 and 5) were enriched in TP53 and/or del(17p) and displayed higher rates of sCNAs and genome alterations. Subtype 1 (13.4%) was marked by WGD and fractured genome (P<0.001 for association with subtypes, subtype 1 is most enriched), along with arm level loss of chromosomes 1p and 9p and MYC amplification. It also contained 6 of 15 M-CLL patients in this study, highlighting WGD as an important mechanism of transformation in M-CLL (Fisher's exact test P=4.6×10⁻³). Subtype 3 (20.6%) was enriched for del(17p) and mutations in TP53, NOTCH1, and IRF2BP2. It contained frequent sCNAs, including del(14q32.11), del(9q), del(15q15.2) [MGA], amp(16q23.2) [IRF8] and del(2q37.1). Subtype 5 (22.7%) similarly displayed high rates of del(17p) and TP53 alterations and frequent sCNAs including del(16q12.1), del(1p35.2) and amp(7p) but lacked NOTCH1 mutation. By contrast, subtypes 2 and 4 showed lower fraction of genome altered (P<0.001 [subtype 2 vs 1, 3, 5]; P<0.005 [subtype 4 vs 1, 3, 5]). Subtype 2 (26.8%) was predominantly marked by tri(12) co-occurring with SPENINOTCH1 and KRAS mutations and contained the highest incidence of novel sSNV RS mutational drivers (DNMT3A, SRSF1). Subtype 4 (16.5%) was marked by SF3B1 and EGR2 mutations on a background of del(13q). The clonally unrelated patients did not cluster together, likely due to diverse features.

To assess whether these RS molecular subtypes displayed distinct transcriptional states, the inventors investigated matched RNA samples from a subset of 36 RS cases. Evaluation of differentially expressed genes between subtypes identified distinct expression signatures defining subtypes 1 (n=25 genes) and 3 (n=188 genes). Subtype 3 was notably enriched in signatures of cell cycle and inflammatory/interferon signaling processes in line with its enrichment for IRF2BP2 mutations. Consistent with these findings and in support of our mutation-based clustering, unsupervised consensus clustering of these RNA-seq data identified 5 transcriptional clusters that we significantly associated with our RS-defined molecular subtypes (Fisher's exact test 5×5 contingency, P=0.038).

The inventors evaluated whether genetically-defined subtypes were associated with clinical outcomes. Subtypes 2 (tri12/NOTCH1/SPEN) and 4 (EGR2/SF3B1) were associated with improved overall survival within clonally related RS (3.3, 11.3, 5.0, 16.7 and 4.0 months for subtypes 1-5, respectively; log-rank P=0.0082) (FIG. 3D). Clonally related cases had shorter median OS (or this endpoint was not reached) (5.8 months) than unrelated ones (56.4 months) (log-rank P=0.0094).

Mutational processes underlying transformation. Evaluation of mutational profiles from the combined CLL and RS WES data revealed signatures of aging and activation-induced cytidine deaminase (AID). The inventors detected a dominant signature of polymerase epsilon (POLE) mutation (Methods) in an unrelated RS case (Pt 30) with deleterious POLE mutation and >2,000 sSNVs in the RS clones. To more deeply assess mechanisms underlying transformation, we analyzed whole-genomes generated from 11 RS trios. WGS-determined phylogenetic trees improved resolution of clones and remained concordant with the WES phylogenies. The CLL and RS clones of the two unrelated RS cases did not share a more distant non-coding evolutionary history, definitively establishing them as unrelated lymphoid malignancies. The inventors further examined 14 WGS from recently published RS patients and demonstrated 2 additional clonally unrelated cases. Mutational analysis of the CLL clones from 10 of 11 evaluable patients revealed signatures similar to the WES analysis. In contrast, the RS clones revealed an expanded breadth of mutational signatures, including prior chemotherapy (SBS17b), reactive oxygen species (SBS18) and defective DNA mismatch repair (SBS44). Kataegis was recently reported in RS, and indeed, the inventors identified a higher number of such regions across 4 of 11 RS genomes compared to paired CLL (Methods).

In inspecting the WGS samples, the inventors observed chromothripsis as a common defining feature of TP53-altered RS genomes, in addition to increased regions of kataegis (with clustered AID-related mutations) and numerous structural variants (SVs). Chromothripsis was observed in regions likely contributing to RS pathogenesis, including 7q21 (CDK6) (Pt 41), 11q13 (CCND1) (Pt 29) and 9p24.1 (PD-L1/L2) (Pt 41) with the majority of regions being patient-specific. Chromothripsis was not observed in WGS of clonally related cases of subtypes 2 and 4 RS (one from each subtype) or unrelated RS (n=2) cases.

### Example 9: Early RS Clones are Detectable in Cell-Free DNA

Given that recurrent copy number events, chromothripsis regions and whole genome doubling strongly associated with RS, the inventors assessed the feasibility of non-invasive detection of RS events through examining serial specimens of cell-free DNA extracted from patient plasma prior to RS diagnosis (Methods) with ultra-low pass whole genome sequencing. Indeed, in the example of Patient 5, RS-specific lesions, including regions of chromothripsis on chromosomes 6 and 16, were readily detected in blood plasma as early as 162 days before RS diagnosis and were not present in the corresponding circulating CLL cells by WES assessment. This patient progressed on multiple CLL-directed regimens prior to ultimate RS diagnosis by tissue biopsy. In another example, RS-specific CNAs and SVs were detected in plasma samples from patient 44, whose RS sample lacked chromothripsis. In three other patients (Patients 9, 32 and 39), CLL clone specific CN changes were found in the copy number profiles of cfDNA with no RS events noted, although plasma samples were all obtained several months prior to RS diagnosis, likely consistent with the rapid progression of this aggressive malignancy. These data not only demonstrate shedding of cfDNA from lymph-node based RT disease and the ability to detect nodal clonal evolution that is not present in corresponding CLL peripheral blood timepoints, but expand on prior data to support potential feasibility for employing a simple genomic assay for early or non-invasive detection of RS as well as for monitoring evolving RS risk lesions.

### Example 10: Dynamics of Transformation at Single-Cell Resolution

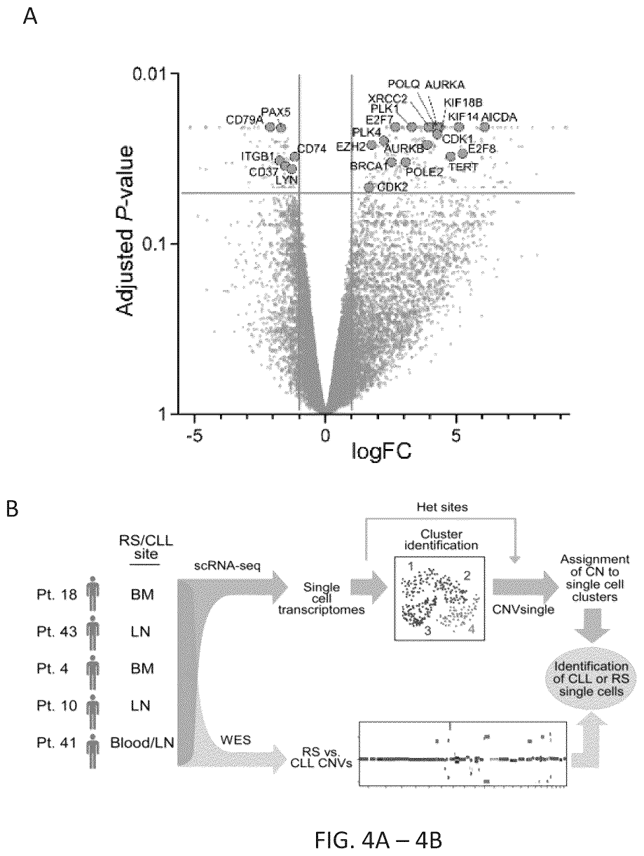

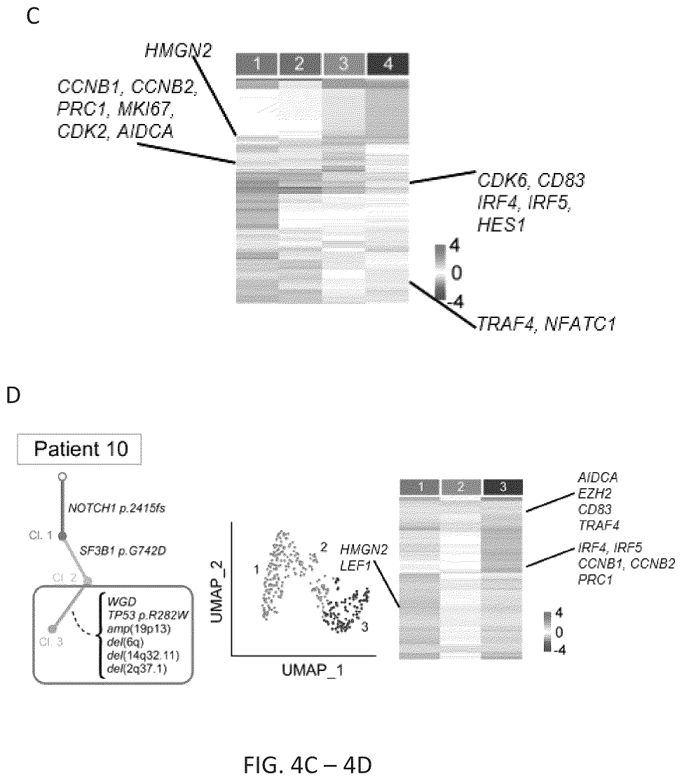

To define the phenotypic changes associated with transformation, the inventors performed bulk RNA-seq analysis on high-purity paired RS and CLL samples from 5 individuals. Comparison of the RS to CLL transcriptomes identified 292 upregulated and 111 downregulated transcripts (|log₂ fold change|>1, adjusted P<0.05) (FIG. 4A). RS contained noticeably more expressed transcripts at higher abundances, as could be expected from large RS cells. Remarkably, the most upregulated transcripts in RS are from genes encoding key enzymes (cyclin dependent kinases (CDK1 and CDK2), aurora kinases (AURKA and AURKB), polo-like kinase (PLK1 and PLK4) and separase (ESPL1)), components (kinesins (KIF genes), centromere proteins) and regulators of mitosis, spindle assembly and cytokinesis (FIG. 4A). Overexpression of these genes has been shown to generate aneuploidy in cancer. The ten most consistently upregulated pathways were all related to mitotic cell cycle, chromosome organization and myc signaling (FIG. 4A), and implicate alterations in these cellular processes in the generation of sCNVs and WGD that are apparent in RS.

To further examine the expression changes and clonal structure of CLL transforming to RS at high-resolution, the inventors performed single-cell RNA-sequencing of RS diagnosis biopsy specimens from 5 patients that contained clonally related RS and CLL cells co-existing within the same tumor microenvironment. After flow cytometric sorting of cells to include viable cells with representative populations of both CLL and RS, droplet based single-cell RNA-sequencing was performed followed by initial clustering and sub-clustering of identified B cells (see, Methods). For the 3 of 5 cases for which BCR data was available, the inventors could confirm the consistent expression of BCR within the cell clusters assigned as CLL or RS, and the Ig clonal relatedness of CLL and RS clusters. Given the numerous RS-defining sCNAs observed from the instant WES data, the inventors devised a novel method to confidently identify the expression clusters representing RS versus CLL clones based on detection of CNV events. The inventors utilized CNVSingle, which is not reference dependent but rather utilizes segmentation of SNP-heterozygous sites to infer the sCNAs across a cluster of individual cells (FIG. 4B, Methods). The inventors found this approach to greatly improve the signal-to-noise ratio over other commonly-used methods (e.g. inferCNV) and could robustly detect tumor-specific CNVs in malignant cells and the absence of CNVs in normal immune cells.

Using a Random Forest classifier on the scRNA sample expression data, the inventors could predict if a cell was CLL or RS with a mean F1 of 0.79 (Methods). Compared to CLL, the RS-identified clones across the evaluated patient samples displayed much higher UMI/cell (i.e. mean 9000 vs. 3193 for Patient 43, p<2.2×10¹⁶, Wilcoxon) and genes/cell (mean 2909 vs 1074, p<2.2×10¹⁶). In line with the bulk RNA data, differential expression analyses revealed greater involvement of mitosis regulators and components in RS compared to CLL, and upregulation of interferon-related transcripts, MYC signaling, proliferation (E2F, G2M targets) and KRAS pathways. Directional trajectories inferred using RNA-velocity supported a transition in cell states from CLL to RS.

Strikingly, CNV assignments appeared to map to distinct transcriptionally-identified cell populations. For example, the lymph node cells of the RS biopsy taken from Patient 43, who developed RS 4.7 months after initiation of front-line targeted therapy (ibrutinib), revealed two groups of clusters that were transcriptionally distinct (clusters 1 and 2 vs clusters 3 and 4). The expression profiles of these cluster groups were consistent with CLL and RS, respectively, although cluster 2 appeared to exhibit some expression changes intermediate between clusters 1 and 3/4 (FIG. 4C). Accordingly, Clusters 3 and 4 expressed both the RS-specific CNA on chromosome 2 as well as many of the clonal RS aberrations mapped to the CLL^{INT} clone 2 from the WES analysis (i.e., CNAs on chromosomes 4, 7, 8, 9 and 13, with fragmentation of some of these regions). In contrast, cluster 1 had a CN profile concordant with the ancestral CLL clone, previously identified in the bulk WES characterization. The CNA profile of cluster 2 presented a higher number of alterations, with a profile showing both the divergent CN changes of WES clone 3 (chr1q gain and its child, chr12q gain) as well as some early CN changes of the WES CLL^{INT} clone 2. The observed CN annotations within these clusters thus support a picture of a continuum of a transition towards RS.

Patient 10 highlighted the rapid evolution of transformation with genomic instability in a lymph node biopsy from a patient with a non-CN altered M-CLL history and highlighted intermediate, or transitional states not captured in bulk genomic analysis. By WES analysis, the inventors had established the lack of CN changes in circulating CLL 50 days prior to transformation compared to the abundant CN changes and WGD in RS cells. By flow cytometric examination of the lymph node cells, both small and large B cell populations were observed, consistent with presence of both CLL and RS cells and clustering of single-cell transcriptomes yielded 3 distinct populations. Cluster 1 displayed a total number of genes per cell consistent with CLL and Cluster 3 expressed a much higher number of genes, consistent with RS. By gene expression profiles, cluster 2 showed an intermediate phenotype between clusters 1 and 2 (FIG. 4D). Despite appearing as a CLL cluster, cluster 1 already demonstrated a path towards stepwise acquisition of genomic instability given the detection of both del(17p) and WGD in this cell cluster. Cluster 2 showed progressive genomic disorder followed by cluster 3, which highly resembled the CN profile of RS identified by WES. These results demonstrate that in this case, chromosome 17p loss and WGD preceded the RS transition, marked by subsequent global copy number shifts and provide insight into the stepwise disorder that is observed in pattern 2 RS.

For patient 4, few malignant B cells were captured but WGD and frequent CNAs were observed, consistent with WES absolute copy number.

In the 2 remaining cases (Pts 18 and 41), the inventors also observed evidence of transitional cell clusters, which possessed expression profiles intermediate to RS and CLL and early CN changes reflecting RS. For Patient 41, the lymph node biopsy and blood cells were flow cytometrically sorted on the basis of cell size and this intermediate transcriptional cell state was found to clearly reside within the FSC-low population characteristic of CLL rather than the FSC-high population of RS. These intermediate cell clusters displayed mean UMI/cell (4381 in CLL vs. 8380 in transitional, 13953 in RS, p<2.2×10⁻¹⁶) and mean genes/cell (1267 in CLL vs 1723 transitional vs 2655 RS, p<2.2×10⁻¹⁶) intermediate to CLL and RS, highlighting the increasing cell size and transcript abundance in these populations. Indeed, upon examination with CNVsingle, these clusters showed acquisition of early RS-specific events, thus highlighting a genetic transitional state. For Patient 18, flow cytometric sorting again clearly distinguished the CLL and RS populations and transcriptional clustering identified distinct early RS (clusters 3 and 4) and RS subclones (cluster 0 containing del(6)) that had been observed in the WES analysis.

### Example 11: Early RS Clones are Detectable in Cell-Free DNA

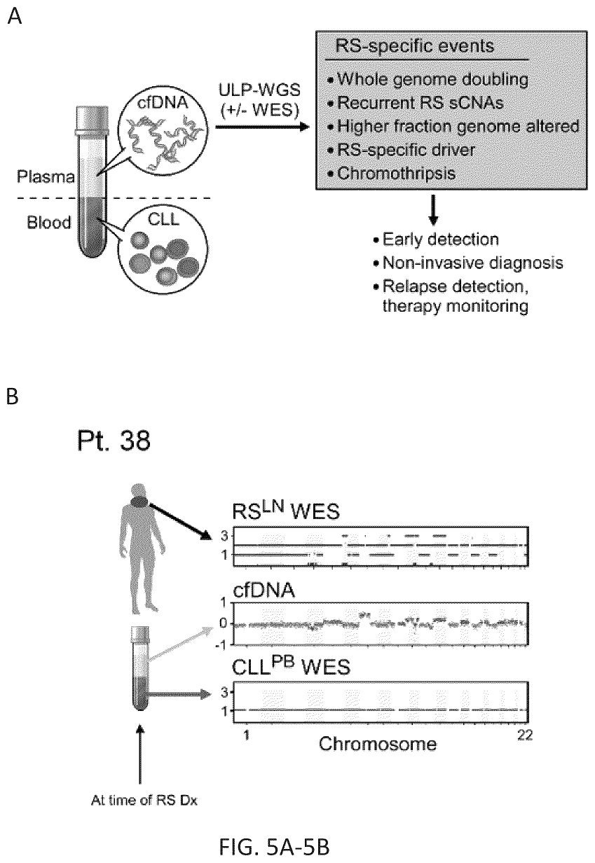

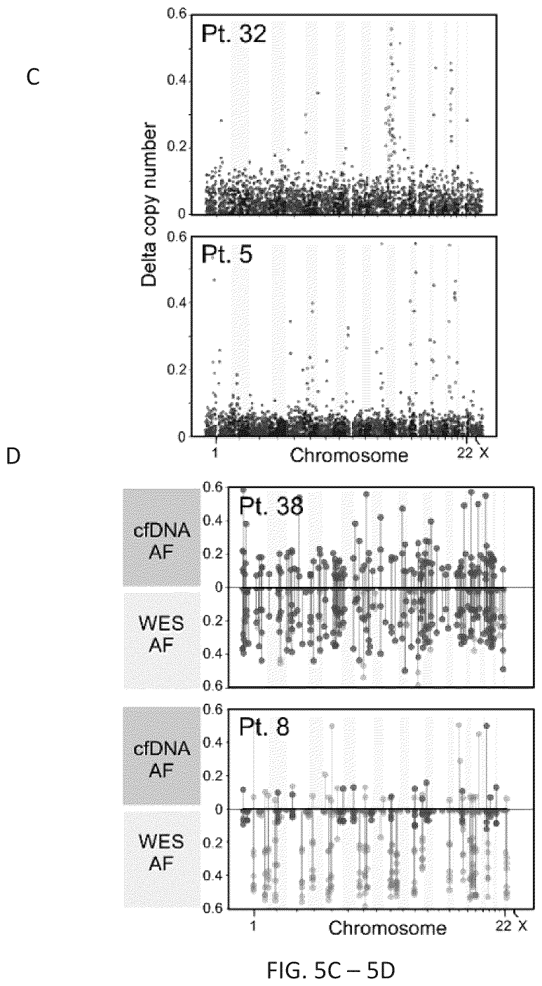

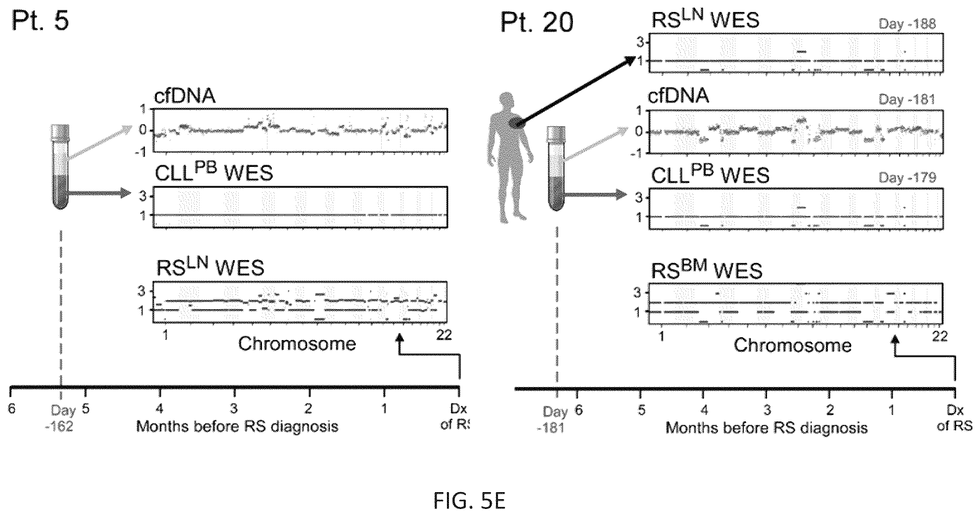

Given the numerous RS-associated genomic features identified by the instant study (recurrent sCNAs, RS-specific sSNVs, WGD, chromothripsis), the inventors assessed the feasibility of non-invasive detection of such RS events through examining cell-free DNA extracted from RS patient plasma at different times relative to the clinical presentation of RS (FIG. 5A). To this end, the inventors evaluated 46 plasma samples for such RS features by ultra-low pass WGS43 that were collected from 24 patients within the three years leading to the time of RS diagnosis and through relapse. Samples from 17 patients were collected at the time of RS disease, eight of which were surrounding initial diagnosis. Ten patients were from our WES-based discovery cohort and their RS characterization served as a positive control for detection of any present RS-specific alterations. Eight of these also had simultaneous (same blood draw), or contemporaneous circulating CLL cells analyzed by WES, thus offering an internally controlled standardized way to evaluate the differing contributions of concurrent nodal vs circulating disease, since the cfDNA includes DNA shedding from both the lymph nodes and the circulating CLL cells.

Evaluation of these samples revealed that each of these RS-associated genomic features were indeed detectable across the cohort, although not all were necessarily found in the same sample. WGD was observed in the cfDNA of Patient 38 at time of RS diagnosis, matching the RS WES profile of the diagnostic lymph node biopsy, while circulating CLL sampled 11 days earlier remained copy number quiet (FIG. 5B). The cfDNA of Patient 44 revealed RS-associated sCNA changes (del(9p), amp(13)), within 17 days prior to histopathologic diagnosis of RS, that were not yet apparent in the CLL blood. cfDNA analysis also highlighted RS emergence in a high-risk CLL patient with del(17p) who had achieved excellent disease response with normalization of circulating WBC count but had expanding lymph nodes. While the cfDNA profile at the start of CLL-directed therapy showed minimal sCNAs, that at time of RS diagnosis (and CLL response) showed abundant new sCNAs, including amplification of chromosomes 8q24 [MYC] and 19p13.2 and del(19p13.3), del(1q42.13) and del(12p13.2). In other patients, chromothripsis was evident in plasma cfDNA at the time of, or preceding RS tissue-based diagnosis (FIG. 5C). Furthermore, for 4 of 4 cases from our discovery cohort in which WES was performed on plasma cfDNA (in addition to low-pass WGS), RS clonal mutations that were absent in circulating CLL cells could be detected (FIG. 5D).

The inventors then asked whether such changes could be detected in advance of biopsy-defined RS diagnosis. For 2 of 7 patients for which plasma was collected 1-10 months prior to RS diagnosis, the inventors could clearly detect RS-associated alterations in the cfDNA, at which time these two patients were undergoing lymphoma-directed therapies for presumed aggressive refractory CLL. In Pt 5, WGD and chromothripsis (chr 6 and 16) were observed in plasma 162 days prior to diagnosis and were absent from the corresponding CLL (FIG. 5E-left). WES of cfDNA further showed presence of RS-specific mutations. In Pt 20, cfDNA 181 days prior to RS diagnosis showed WGD and sCNAs not present in the corresponding CLL blood sample (day-179) or nodal biopsy from the prior week (FIG. 5E-right).

Finally, the inventors queried the potential for cfDNA analysis to detect early disease relapse. The inventors considered two RS patients who had achieved a state of minimal CLL involvement following allogeneic hematopoietic stem cell transplantation (allo-HSCT), and subsequently relapsed with nodal RS. For Patient 112, cfDNA obtained immediately following HSCT initially lacked evidence of genomic instability or RS copy-number events, corresponding to observed disease response. By 83 and 162 days post-HSCT however, cfDNA analysis revealed new sCNAs, and thus increased fraction genome altered (FGA), consistent with nodal disease evolution (FIG. 5F). Ultimately, biopsy-confirmed RS relapse was diagnosed on post-HSCT day 187. With subsequent therapy, the patient achieved a CR and the RS-associated cfDNA changes were resolved. Pt 111 achieved durable remission following allo-HCST for CLL and before developing RS two years later. Post transplant, prior to RS diagnosis, the patient intermittently had elevated FGA in plasma, which subsequently resolved following RS therapy, suggesting ongoing nodal disease that eventually became RS.

Across all samples, we evaluated the relationship between RS-associated changes in cfDNA relative to the time of RS diagnosis (FIG. 5G). By the measure FGA, the highest levels were observed in samples collected at the time of RS diagnosis (median value=0.2, n=8), with decreasing ratio observed in the 1-10 months before diagnosis (median value=0.041, n=7), and even lower ratio in samples collected greater than 10 months prior to RS (median value=0.006, n=4) (FIG. 5G). In 7 cases (time of RS and leading up to RS), the measured FGA exceeded all values seen in high-risk relapsed/refractory CLL cases (n=14 samples from 5 patients). Of the 8 patients with cfDNA available at the time of biopsy-proven diagnosis, the inventors confidently discerned RS-specific lesions in 6 (75%) using strict criteria (Methods). Altogether, these data not only demonstrate consistent shedding of cfDNA from RS disease44 and the ability to detect nodal clonal evolution, but support the feasibility for employing a simple and inexpensive genomic test for non-invasive early detection of RS, including a “single vial” assay when CLL cells serve as control for RS cfDNA obtained in the same blood draw.

### Example 12

We now squarely reside in the era of expanded therapeutic capabilities to address cancer, ranging from targeted pathway inhibitors to immunotherapy. Each of these treatments apply strong selective pressure on the heterogeneous cancer cell populations; thus, gaining understanding of the molecular basis of clonal escape has risen to a high level of priority. Genomic characterizations provide the foundation for more sensitive early detection of disease progression, more precise diagnosis and broader options for therapy, yet heterogeneity and admixed populations provide analytical challenges, especially in the setting of a histologic transformation, which results in two separate co-existing malignancies. Perhaps one of the most intriguing examples of rapid disease evolution with histologic switch is Richter Syndrome. For decades, the diagnosis of RS has relied on morphologic characterization of aggressive lymphoma within the context of concurrent or known history of CLL. In the instant disclosure, through the implementation of advanced genomic analytic approaches that are able to distinguish between the RS and CLL clones, and through integration of exome, genome and transcriptome data to the largest series of paired CLL and RS specimens to date, the inventors have defined the distinct molecular events that precede and define the RS transition.

Of the new insights gained from this study, the first is the identification of novel driving events in RS, distinct from the antecedent CLL. These include mutations affecting splicing, DNA damage, immune evasion, and interferon signaling and CNAs impacting MYC signaling, cell cycle regulation and epigenetic drivers. The instant study highlights major differences between RS and de novo DLBCL despite several shared driver events. Noticeably the N1 subtype of DLBCL does carry more similarity to RS pattern 4, highlighting potential shared biology.

Second, for clonally related RS, the inventors identified heterogeneous patterns of co-occurrence of genetic lesions, delineating 4 distinct preferred paths of evolution taken by each subgroup, that yet convergently impact the trio of pathways of DNA damage, MYC and NOTCH signaling. Notably, the identified patterns appeared to have prognostic significance. In particular, the inventors uncovered a previously-unrecognized subset of RS marked by WGD during transformation that is particularly associated with massive DNA instability (chromothripsis and kataegis). CLL was among the first cancers described to demonstrate chromothripsis (cite Campbell), which has been reported to preferentially occur in U-CLL rather than M-CLL. In other recent work, near tetraploidy was identified as an RS risk factor, and the instant single-cell data (Pt 10) demonstrates clearly how rapid evolution to RS can occur from CLL displaying this unstable state. This tetraploid state could result from mitosis defects, as suggested by the instant RNA expression data, and WGD may confer potential vulnerabilities to chemotherapeutics. In line with this, the inventors observed better overall survival for patients with these aberrations. Interestingly, the majority of M-CLL that transformed to clonal related RS occurred in this category and the instant signature and gene expression analysis supports dysregulated AID as a driver of RS.

Third, given the recurrent copy number events, chromothripsis regions and whole genome doubling strongly associated with RS, the inventors demonstrate that cell-free DNA through ultra-low pass WGS might provide an earlier and non-invasive detection opportunity, which is highly clinically relevant in this rapidly progressing cancer. Ultra-low pass WGS is a cost effective assay, and could be impactful in identifying CLL patients with RS and/or high-risk nodal lesions, with implications for therapy selection and clinical monitoring.

Fourth, the instant co-occurrence analysis and single cell analysis gave the inventors an opportunity to consider the order of acquisition of events leading to transformation. In general, it was observed that genetic changes often precede the final transcriptional shift to RS. The inventors identified examples of RS occurring from clonal evolution of aggressive CLL clones and acquisition of successive CN changes followed by expression shift, as in pt 43. Alternatively, it may arise very rapidly in the setting of new 17p deletion and rapidly ensuing WGD and genome instability/chromothripsis, even in an otherwise low risk M-CLL patient. The inventors find transitional states in several patients where intermediate copy number changes were detected, marked by gene expression and cell proliferation, which represented states in between WES clones, thus allowing us to trace and gain insight into RS development.

Finally, the inventors confirmed that a substantial portion of RS disease is unrelated to the co-occurring CLL. The instant disclosure demonstrates that by exome, and even genome level analysis, clonal unrelated RS appears to arise de novo, lacking any shared distant genetic history with the co-existing CLL. Furthermore, it was observed these cases tend to lack TP53 and NOTCH1 alterations, and are enriched in M-CLL, providing novel clinical and molecular insights that may help identify these patients with a more favorable prognosis for further testing.

In this cohort, prior treatment history, including targeted inhibitor agent exposure, did not seem to be associated with any particular RS pattern or trajectory. This suggests that while the inventors did see prior therapy mutational signatures in some patients, that RS may evolve similarly both in the absence of therapeutic selective pressure and/or novel agent exposure.

The impact of the present work is to provide an advanced novel molecular framework for understanding the biology of transformed CLL, linking these distinct categories to clinical outcomes, that is extendable for dissecting this process in lymphoma. This comprehensive evolutionary tracing enables a molecular definition of RS that will provide an opportunity to guide identification of RS in supplement to expert morphological and clinical diagnosis. RNA or cfDNA may be an emerging opportunity for both early diagnosis and identification of RS-specific changes.

### Example 13: Methods

Patient tumor and normal sample collection and processing. CLL, RS, and normal germline (i.e. non-tumor) samples were collected through sample collection protocols from Dana-Farber Cancer Institute (DFCI), University of Ulm in Germany, the CLL Research Consortium (including UCSD, Mayo Clinic, MD Anderson Cancer Center) and the French Innovative Leukemia Organization (FILO) group. All biospecimen collection protocols were conducted in accordance with the principles of the Declaration of Helsinki and with the approval of the Institutional Review Boards (IRBs) of the respective institutions. The CLL DNA studied originated predominantly from peripheral blood mononuclear cell (PBMC) samples (n=100, 89%), while RS DNA was extracted primarily from lymph nodes samples (n=79, 79%), preserved in formalin and embedded in paraffin (FFPE) (n=29, 29%) or fresh frozen tissue (n=70, 70%).

RS samples. RS samples were collected from bone marrow, lymph node, lymphoid tissue or peripheral blood at the time of RS diagnosis and/or relapse and included both fresh frozen and FFPE samples. Freshly collected tissue samples were disaggregated and processed by GentleMACs digestion (Miltenyi Biotec) prior to cryopreservation with FBS/10% DMSO and storage in liquid nitrogen or directly stored as whole tissue blocks in −80° C. and then in liquid nitrogen. Blood and bone marrow specimens were isolated by Ficoll/Hypaque density gradient centrifugation prior to cryopreservation with FBS/10% DMSO and stored in liquid nitrogen. For viably frozen samples of low purity (<30% tumor), RS cells were isolated by fluorescence activated cell sorting (FACS) using an Aria II instrument (Becton Dickinson) based on dual expression of CD5+ and CD19+ on cells with increased forward scatter (FSC) (Biolegend, CD5-FITC cat #364022, CD19-PE-Cy7 cat #302216). For FFPE specimens, samples from each submitting collaborating center were reviewed for >50% purity prior to submission for sequencing.

CLL samples. CLL tumor samples were obtained from peripheral blood both prior to RS diagnosis and at the time of or after RS diagnosis. Samples with higher CLL purity (WBC >25×10³/microliter or ALC >20×10³/microliter) were processed without CD19 selection, and CLL cells were isolated by Ficoll/Hypaque density gradient centrifugation and then cryopreserved with FBS/10% DMSO and stored in vapor phase liquid nitrogen until the time of analysis. Samples with WBC <25,000/μL or ALC <20,000/μL underwent CD19 selection (RosetteSep Human B-cell enrichment, Stem Cell Technologies) or as previously described (PMID: 26466571), or FACS sorting to enrich for CD5+CD19+ populations.

Germline samples. Sources of non-tumor germline DNA included saliva (Oragene Discover [ORG500 or ORG600] kit, DNA Genotek), bone marrow at the time of complete remission in vitro expanded T cells. For the latter, mononuclear cells were stained with anti-CD19 (PE-Cy7, Biolegend cat #302216,), anti-CD5 (BV421, Biolegend cat #300626) and anti-CD4 (FITC, Biolegend cat #300506) or anti-CD3 (Pacific Blue, Biolegend, cat #300330) antibodies. CD19-CD4+ or CD19-CD3+ cells were collected by FACS (Aria II, BD). The cells were plated and expanded in vitro with Dulbecco's Modified Eagle Medium (Gibco) or RPMI (Gibco) containing phytohemagglutinin (PHA) (1.5:100), IL-7 (20 ng/mL), IL-2 (100 U/mL), 10% human serum and beta-2-mercaptoethanol (1/1000). After one week, a new PHA stimulation was performed if the target numbers of cells (>200,000) were not met. Assessment of CD4+ cell purity was checked by flow cytometry at the end of culture.

### Genomic DNA Sequencing

Whole-exome sequencing (WES). Samples were processed and sequenced at the Broad Institute (Cambridge, MA). For these fresh blood and bone marrow samples and cryopreserved suspension cells, genomic DNA and RNA was extracted per manufacturer's recommendations (Qiagen). DNA was quantified in triplicate using a standardized PicoGreen® dsDNA Quantitation Reagent (Invitrogen) assay. The quality control identification check was performed using fingerprint genotyping of 95 common SNPs by Fluidigm Genotyping (Fluidigm, San Francisco, CA). Library construction from double-stranded DNA was performed using the KAPA Library Prep kit, with palindromic forked adapters from Integrated DNA Technologies. Libraries were pooled prior to hybridization. Hybridization and capture were performed using the relevant components of Illumina's Rapid Capture Enrichment Kit, with a 37 Mb target. All library construction, hybridization and capture steps were automated on the Agilent Bravo liquid handling system. After post-capture enrichment, library pools were denatured using 0.1N NaOH on the Hamilton Starlet. Cluster amplification of DNA libraries was performed according to the manufacturer's protocol (Illumina) using HiSeq 4000 exclusion amplification chemistry and HiSeq 4000 flowcells. Flowcells were sequenced utilizing Sequencing-by-Synthesis chemistry for HiSeq 4000 flowcells. The flowcells were then analyzed using RTA v.2.7.3 or later. Each pool of whole-exome libraries was sequenced on paired 76 cycle runs with two 8 cycle index reads across the number of lanes needed to meet coverage for all libraries in the pool. Output from Illumina software was processed by the Picard data-processing pipeline to yield BAM files containing demultiplexed, aggregated aligned reads. Standard quality control metrics, including error rates, percentage-passing filter reads, and total Gb produced, were used to characterize process performance before downstream analysis.

Twenty seven samples were processed and sequenced at University of Ulm Germany using Agilent baits. Eleven samples were processed (SureSelect QXT Agilent kit) and sequenced on a HiSeq 1000 instrument at the University of Nancy, France.

A subset of the WES data had reduced coverage in the GC-rich region of NOTCH1. For these, targeted deep sequencing of the NOTCH1 3′ UTR was performed to cover the NOTCH1 3′ UTR hotspot mutation at position chr9:139390152T>C and surrounding sequence.

### Whole-Genome Sequencing (WGS)

Preparation of libraries for cluster amplification and sequencing (PCR-Free). 350 ng of genomic DNA in 50 μL of solution was processed by fragmentation through acoustic shearing (Covaris focused ultrasonicator), targeting 385 bp fragments, and additional size selection was performed using a SPRI 80 cleanup. Library preparation (Hyper Prep without amplification module, KAPA Biosystems, #KK8505) was performed as above for WES. Libraries were then quantified using quantitative PCR (KAPA Biosystems) with probes specific to the ends of the adapters, normalized to 1.7 nM, and then pooled into 24-plexes.

Preparation of libraries for cluster amplification and sequencing (PCR-Plus). An aliquot of genomic DNA (100 ng in 50 μL) was used as the input into DNA fragmentation. Shearing was performed as described above in the PCR-free procedure. Library preparation was performed using a commercially available kit provided by KAPA Biosystems (KAPA Hyper Prep with Library Amplification Primer Mix, product KK8504), and with palindromic forked adapters using unique 8-base index sequences embedded within the adapter (Roche). The libraries were then amplified by 10 cycles of PCR. Following sample preparation, libraries were quantified using quantitative PCR (KAPA Biosystems) with probes specific to the ends of the adapters. This assay was automated using Agilent's Bravo liquid handling platform. Based on qPCR quantification, libraries were normalized to 2.2 nM and pooled into 24-plexes.

Cluster amplification and sequencing (NovaSeq 6000). Sample pools were combined with NovaSeq Cluster Amp Reagents DPX1, DPX2 and DPX3 and loaded into single lanes of a NovaSeq 6000 S4 flowcell cell using the Hamilton Starlet Liquid Handling system. Cluster amplification and sequencing occurred on NovaSeq 6000 Instruments utilizing sequencing-by-synthesis kits to produce 151 bp paired-end reads. Output from Illumina software was processed by the Picard data-processing pipeline to yield CRAM or BAM files containing demultiplexed, aggregated aligned reads. All sample information tracking was performed by automated LIMS messaging.

Circulating DNA sequencing. Whole blood was collected by routine phlebotomy. Plasma was separated within 1-4 days of collection through two density centrifugation steps and stored at −80° C. until cell-free DNA (cfDNA) extraction (QIAsymphony DSP Circulating DNA Kit, QIAGEN), which was performed according to the manufacturer's instructions. Library preparation was performed (KAPA HyperPrep Kit with Library Amplification, KAPA Biosystems) using duplex UMI adapters (IDT), starting with 2-5 cc of plasma. Samples were normalized and pooled using equivolume pooling, with up to 95 samples per pool. Cluster amplification was performed according to the manufacturer's protocol (Illumina) using Exclusion Amplification cluster chemistry and HiSeqX flowcells. Flowcells were sequenced on v2 Sequencing-by-Synthesis chemistry for HiSeqX flowcells. The flowcells were then analyzed using RTA v.2.7.3 or later. Each pool of ultra-low pass whole genome libraries was run on one lane using paired 151 bp runs.

### Sequence Data Processing and Analyses

WES/WGS alignment and quality control. All DNA sequence data was processed through Broad Institute pipelines, such that data from multiple libraries and flow cell runs were combined into a single BAM file. This file contains reads aligned to the human genome hg19 genome assembly (version b37, using BWA-MEM [version 0.7.15-r1140]) provided by the Picard and Genome Analysis Toolkit (GATK) developed at the Broad Institute, a process that involves marking duplicate reads, recalibrating base qualities and realigning around indels.

WES analysis. Sequences were analyzed by the WES Characterization Pipeline, in which aligned.bam files were inputted into a standard WES somatic variant-calling pipeline that included MuTect (PMID: 23396013) for calling somatic single nucleotide variants (SSNVs), Strelka2 (PMID: 30013048) for calling small insertions and deletions (indels), deTIN (PMID: ERV481829941871) for estimating tumor-in-normal (TiN) contamination, ContEst (PMID: 21803805) for estimating cross-patient contamination, AllelicCapSeg (PMID: 26192918) for calling allelic copy number variants, and ABSOLUTE (PMID: 22544022) for estimating tumor purity, ploidy, cancer cell fractions, and absolute allelic copy number. Artifactual variants were filtered out using a token panel-of-normals (PoN) filter (PMID: 29596782), a blat filter, and an oxoG (PMID: 23303777) filter. For tumor samples without a matching normal control, a robust “no-normal” pipeline was used, as previously described (PMID: 29713087). Several FFPE samples exhibited lower DNA quality, resulting in noisier profiles with standard methods. For these samples the inventors applied an additional filtering technique of identifying the most correlated targets across a set of FFPE samples and performing tangent normalization on samples that showed consistent behavior, thus excluding artifactual copy-number targets.

WGS Analysis. Due to the large amount of computational resources required to efficiently process cancer whole genomes, the inventors ran their analysis pipelines on an elastic high performance computing (HPC) cluster on Google Cloud VMs, comprising thousands of CPU cores. For the WGS validation cohort, BAM files were directly obtained from the UK group. Since the BAM files were not aligned to the exact genome reference expected by the pipeline, the inventors realigned them to the Broad Institute's build of hg19 (known as b37: at gatk.broadinstitute.org/hc/en-us/articles/360035890711-GRCh37-hg19-b37-humanG1Kv37-Human-Reference-Discrepancies). Out of the 17 sample trios obtained from the UK group, 14 samples completed WGS (3 failed due to data quality and realignment issues). The standard pipeline as previously described was applied to these FFPE samples except for detection of sCNAs. Formalin damage results in extremely noisy read coverage profiles, confounding traditional copy number segmentation pipelines. To mitigate this, the inventors applied a modified sCNA calling method that relies on segmentation of allelic imbalance at germline het sites (as opposed to segmentation of total coverage) as its primary signal. Although total coverage is extremely noisy, the fraction of reads supporting alternate versus reference alleles at heterozygous sites is undistorted, allowing for clean allelic imbalance segmentation. Within each segment of allelic imbalance, we binned total coverage on a megabase scale, which is coarse enough to average over formalin-induced coverage fluctuations, which typically manifest as sharp coverage spikes at the 10-100 kilobase scale. SV and phylogenetic analysis were completed for 12/14 samples.

Structural variant calling. For structural variation (SV) detection, the instant pipeline integrates evidence from three structural variation detection algorithms (Manta, SvABA and dRanger) to generate a list of structural variation events with high confidence from whole genome sequencing data. The inventors followed the three SV detection tools with BreakPointer to pinpoint the exact breakpoint at base-level resolution. Breakpoint information was aggregated per sample to identify: (i) balanced translocations, which were defined as those with breakpoints on reverse strands within 1-kb of each other; (ii) inversions supported on both ends; (iii) complex events, based on the number of clustered events within 50-kb of each other. Breakpoints were annotated by intersection with the lists of CLL driver genes and significant sCNA regions, as well as with genes in the COSMIC Cancer Gene Census (v90).

Identification of regions of kataegis and chromothripsis. In the whole genome samples, kataegis regions were defined by genomic regions with at least 6 mutations within 2 standard deviations of the median chromosomal intermutational distance, as previously described (PMID: 32025018). For FFPE samples, to account for increased background sequencing artifacts, the inventors considered only mutations with VAF>0.15. Regions of chromothripsis were identified based on integrated evaluation of rainfall plots, allelic CN plots and SV calls. To identify samples with regions of potential kataegis/chromothripsis in WES data, a model was applied based on a definition previously established by Alexandrov (PMID: 32025018), namely loci with at least 3 consecutive mutations, each with intermutational distance <1,000 bp. In this way, 3 of 5 whole-genomes with kataegis/chromothripsis were also detected in the WES data; furthermore, 7 potential cases were detected in the WES-only cohort.

Determining evolutionary relationships between RS and CLL and identifying RS specific genetic alterations. To identify RS clones separately from CLL clones and to infer phylogenetic and evolutionary relationships, the inventors applied PhylogicNDT (cite phylogic paper and PMID: 31142838). The PhylogicNDT suite of tools was used to generate posterior distributions on cluster positions and mutation membership to calculate the ensemble of possible trees that support the phylogenetic relationship of detected cell populations. Through applying this component tool across a set of CLL and RS samples per patient, the most likely tree was identified using probabilistic modelling and thus parent-child relationships among clones. Furthermore, all mutations are assigned to clones based on the match of mutational and clone CCF distribution. The RS clone was then genetically defined as a novel emerging clone first detected in the RS sample and absent in a preceding CLL sample. In the rare cases without close antecedent CLL samples, RS clones were conservatively identified from distal tree branches as well as through integrating available information on RS purity from pathology/clinical assessment. If a shared CLL historical clone was identified between samples, the RS was determined to be clonally related. If a shared clone was not identified across samples, the RS was determined to be clonally unrelated.

In WGS Phylogic results only, clusters with fewer than 20 mutations were removed along with clusters with low CCF and evidence of clustering in certain genomic regions.

Mapping CN alterations to RS and CLL clones. Once clones were identified in serial samples, the inventors mapped subclonal sCNVs using PhylogicNDT CopyNumber2Tree to identified RS and CLL clones on a per patient basis (across all patient samples). Posterior probability was calculated based on CN profiles and allele-fraction distributions of heterozygous SNP sites across samples to assign likelihood of each event to belong to a cancer cell fraction clone. In this manner, the RS and CLL specific clonal events (both sSNV and sCNV) could be identified

Identification of significantly mutated genes in RS and CLL clones. To identify candidate cancer genes using the mutation calls from WES, the inventors ran MutSig2CV to identify driver genes from the filtered WES Mutation Annotation Format (MAF) file of both the RS history (related CLL clones and RS clones), thus excluding divergent CLL clones, and identifying the complete mutational spectrum contained within RS cells and clones, to identify RS specific mutations, including newly arising in RS. A stringent manual review was conducted using the IGV to review the mutations in the driver genes and further exclude low evidence calls. To further improve the power to detect known variants, the inventors ran MutSig2CV using restricted hypothesis testing through utilizing a comprehensive well-annotated list of CLL drivers and de novo DLBCL drivers (PMID: 29713087).

Identification of recurrent focal and arm level copy number events in RS. For detecting somatic copy number alterations (sCNAs) the inventors used the GATK4 CNV pipeline (the GitHub website), which involves the CalculateTargetCoverage, NormalizeSomaticReadCounts, and Circular Binary Segmentation (CBS) algorithms for genome segmentation. For a subset of the cohort, which was typically FFPE-derived, CN profiles were improved, as described above. In order to identify candidate sCNA drivers (genomic regions that are significantly amplified or deleted), the inventors then apply GISTIC 2.0 on the RS samples, both before and after subtracting the CLL sample segment changes, to produce a list of candidate RS sCNA driver regions. In parallel, the inventors then examined the antecedent CLL sCNA drivers through GISTIC. Significant events were reported with a q value threshold of 0.1. A force-calling process was applied to identify the presence/absence of each sCNA driver event across tumor samples (the GitHub website). Finally, all filtered sCNA drivers were manually reviewed using IGV to exclude drivers that are based on sCNA events with low supporting evidence.

To compare RS drivers identified with previously reported CLL and DLBCL datasets, a 2 sided exact binomial test was performed with Benjamini-Hochberg multiple test correction to compare both CLLanc mutational and sCNA frequencies with CLL reference data, as reported by Knisbacher et al, 2021 (n=1063), and RS event frequencies with DLBCL (PMID: 29713087) (n=304). Event frequencies were compared when an event was detected in both sample sets.

Immunogenetic analysis. To determine the clonal relationships between CLL and RS, the inventors inferred the DNA sequences of immunoglobulin genes (V (D) J gene usage and the CDR3 sequences) from WES/WGS data from each RS sample and the most proximal paired CLL sample. The tool IgCaller v1.1 (the GitHub website github.com/ferrannadeu/IgCaller) was applied for identifying IGH gene rearrangement and sequence reconstruction. The inventors reported the productive and best scoring IGH rearrangements based on IgCaller judgment and cross-validated by analyzing Fasta sequences of IGH region with IMGT-vquest. The inventors used the tool MiXCR v3.0.10 on WES, WGS and RNA-seq data to quantitate BCR clonotypes and reconstruct the CDR3 sequences. In cases where no IGH rearrangement was revealed with IgCaller the inventors looked at the IGL and IGK locus based on MiXCR output. The inventors assessed the IGHV mutational status based on the percentage of identity of the IGHV region to the germline sequence (using 98% as the cutoff).

Signature analysis. Mutational signatures were determined using SignatureAnalyzer (the GitHub website github.com/getzlab/getzlab-SignatureAnalyzer). Using a Bayesian version of NMF without requiring the number of signatures K, SignatureAnalyzer probabilistically infers K through the automatic relevance determination technique and returns highly interpretable and sparse representations for both underlying mutational signature profiles and patient attributions that strike a balance between data fitting and model complexity. Finally, to further interpret the results, the inventors compared the identified signatures with those in COSMIC (v3.2) based on cosine similarity via manual review.

Bulk RNA-sequencing and data analyses. High-quality RNA from CLL/RS pairs was extracted, as previously described (PMID: 26466571). Total RNA was quantified using the Quant-iT™ RiboGreen® RNA Assay Kit and normalized to 5 ng/μl. Following plating, 2 μL of ERCC controls (using a 1:1000 dilution) were spiked into each sample. An aliquot of 200 ng for each sample was transferred into library preparation which uses an automated variant of the Illumina TruSeq™ Stranded mRNA Sample Preparation Kit. This method preserves strand orientation of the RNA transcript. It uses oligo dT beads to select mRNA from the total RNA sample, followed by heat fragmentation and cDNA synthesis from the RNA template. The resultant 400 bp cDNA then goes through dual-indexed library preparation: ‘A’ base addition, adapter ligation using P7 adapters, and PCR enrichment using P5 adapters. After enrichment, the libraries were quantified using Quant-iT PicoGreen (1:200 dilution). After normalizing samples to 5 ng/μL, the set was pooled and quantified using the KAPA Library Quantification Kit for Illumina Sequencing Platforms. The entire process was in a 96-well format and all pipetting is done by either Agilent Bravo or Hamilton Starlet. Pooled libraries were normalized to 2 nM and denatured using 0.1 N NaOH prior to sequencing. Flowcell cluster amplification and sequencing were performed according to the manufacturer's protocols using either the HiSeq 2000 or HiSeq 2500 instrument. Each run generated a 101 bp paired-end with an eight-base index barcode read. Data was analyzed using the Broad Picard Pipeline, which includes de-multiplexing and data aggregation.

Data analyses. RNA-seq reads were aligned to the human reference genome hg19 using STAR (v2.4.0.1) and TPM (Transcripts Per Kilobase Million) value was used to measure gene expression. Differentially expressed (DE) genes were assessed using DESeq2 with FC>2 and FDR<0.05 as a cut-off. To ensure robustness of the analysis, for the 5 pairs of RS and CLL samples analyzed, DE genes were recalculated iteratively, each time leaving out one sample pair; the highest FDR value amongst all comparisons was selected for each gene. Pathway analysis was performed using GSEA.

### Single-Cell RNA-Sequencing and Analysis.

Sample preparation. For suspension samples with admixture of both CLL and RS cells, cells were thawed by drop-wise addition of warmed media (RPMI 10% FCS) and stained with antibodies (Biolegend CD5 FITC cat #364022, CD19 PE-Cy7 cat #302216, CD3 PB cat #300330) and a viability marker (Biolegend 7-AAD cat #420404 or Zombie Violet cat #423114) before resuspension in PBS-0.04% BSA (Ultrapure NEB/Invitrogen). For Patients 19 and 41, viable CD5+CD19+ cells were sorted into RS and CLL fractions by size based on the increased forward scatter (FSC) of RS cells (BD FACS Aria II). For Patients 43, 4 and 10, viable cells within the lymphocyte gate were sorted for analysis.

Sequencing. Five to ten thousand single-cells per specimen underwent transcriptome sequencing (Chromium Controller, 10× Genomics) according to the manufacturer's instructions, using either 3′ v2 kit (Patients 19 and 41) or 5′ v2 kit with BCR and TCR sequencing (Patients 43, 4 and 10). Each flow sorted fraction was run as a separate lane on the same chip with duplicates included for some tumor samples. Libraries were pooled and sequenced on HiSeqX or NovoSeqS4 (Illumina).

Data processing of scRNA-seq libraries. For Patients 4, 10 and 43, scRNA-seq and scV(D)J) reads were processed and aligned to the Hg19 reference genome. All data were filtered using the Cell Ranger pipeline (v2.1.1 for Patient 41; v2.0.0 for Patient 19, and v3.0.2 for Patients 4, 10 and 43). Filtered feature-barcode matrices containing detected cellular barcodes were utilized in further analyses for Patients 19 and 41. Background, or ambient, RNA was removed using CellBender with the exception of Patient 41.

Data from each patient was analyzed, processed and clustered separately using Seurat (v3.1.4) with a standard workflow. QC filtering was applied to remove cells with fewer than 500 UMIs, >50,000 UMIs or more than 10% mitochondrial mapped reads. Raw counts were log-normalized to counts per million (logCPMs), variable genes were detected and normalized data was centered and scaled. Principal component analysis (PCA) was performed, followed by an initial clustering with resolution set at 0.8. Clusters were visualized by UMAP. Potential doublets were detected using DoubletFinder (v2.0.2). DoubletFinder was run at default value for the number of artificial doublets generated (pN=0.25), and optimal pK was identified for each patient sample. Identified doublets were removed and data was reprocessed and reclustered.

For Patient 41, initial clusterings were partially driven by expression cell cycle genes, and for these patients the inventors performed regression on these genes first using Seurat's CellCycleScoring function to generate module scores for each of the three phases and then using these as features to regress out in the ScaleData function. For Patient 41, the inventors further corrected for potential batch effects between samples from different tissues (lymph node and peripheral blood) using Seurat's standard workflow for data integration.

From the cleaned data, the inventors performed clustering and identified B cell clusters; these were further subclustered for additional analysis of malignant B cells. For Patients 4, 10 and 43, additional TCR and BCR clonotype information was utilized to confirm B cell clusters in addition to the use of standard B cell markers (CD19, CD20, IGLL5, CD79A, CD79B) and the absence of T/NK/Myeloid markers (CD3, CD4, CD8, CD56, CD14, CD16, CD33). The inventors reanalyzed these subsets, and tested resolutions ranging from 0.4 to 1 (interval=0.1) followed by Clustree (v0.4.2) in order to identify stable clusters prior to downstream analysis. UMI/cell and genes/cell for each cluster were calculated with Seurat and the mean values across CLL and RS clusters were compared using a Wilcoxon test.

Inferred copy number across single cells (CNVsingle). The inventors applied CNVsingle to the above processed Seurat objects. In brief, CNVsingle utilized normalization from matched PBMC derived B-cell profiles followed by Savitzky-Golay noise reduction. These profiles alongside the per cell allele counts across common heterozygous SNP sites identified in the samples were utilized by a Hidden Markov Model running in allele specific mode on subsets of cells. Thus CNVsingle provides allele-specific copy number profiles for all malignant cell clusters. It was validated that different types of normal cells provided copy-neutral profiles. Single cell derived allelic copy number across clusters was compared to WES CN profiles and found to be highly concordant. These profiles were then used to identify clusters as CLL, RS or transitional and cluster identities were used for subsequent differential expression testing.

Differential expression testing. To evaluate differential gene expression between defined CLL and RS clusters, a standard normalization per cell was first performed. To avoid issues with zeros and negative values, the distribution was shifted to set minimum value to numpy (v 1.20.3) epsilon. The ranking for the highly differential genes in each cluster was computed via t-test using the rank genes groups scanpy (v 1.8.2) function. The ranked gene list from the DE analysis for RS clusters in each scRNAseq sample was submitted to pre-ranked GSEA to analyze the HALLMARK and GO Biological Process (GOBP) pathways (1,000 permutations, weighted enrichment statistics, MSigDB v7.4).

Velocity Analysis. RNA inference of directional trajectories was performed with scvelo (v 0.2.4—with fit_connected_states=False) with dynamical model on the normalized data. Spliced and unspliced reads were computed via velocity (v 0.17.17) (PMID 32747759). The result of the model was then used to estimate gene latency, which represents the cell's internal clock and is based only on its transcriptional dynamics. The root key parameter has been computed via the cellrank (v 1.5.0) library.

ML/AI Analysis. The inventors used a machine learning (ML)/artificial intelligence (AI) approach to differentiate between CLL and RS cells in normalized LN samples. To perform this prediction task, a Random Forest (in sklearn) was trained using the gene expressions for some group of patients, and predicting the cells of other patients. This task was performed 20 times, obtaining an average F1 of 0.79.

The inventors used a Random Forest (RF) approach to differentiate between CLL and RS cells in the single cell data. Data was preprocessed using the same cell/gene filtering as in the DE analysis. To reduce the impact of cell size differences between CLL and RS, we performed a z-score normalization per cell. We trained an RF (n_estimators=1000, sklearn v1.0.1) on samples (lymph nodes; LNs) from Pts 10 and 43 and predicted on cells from Pts 41 (LN or peripheral blood [PB] samples) and 18 (bone marrow) whose cell labels were determined by FACS sorting described earlier. We ran the RF 20 times and obtained a mean±σ of 0.92±0.01 when looking at only the LN sample in the test set to avoid any potential microenvironment differences. When we included the PB sample in the test set, the F1 only slightly decreased to 0.86±0.11; while also adding in Pt 18 yielded an F1 of 0.66±0.01. The decrease in F1 score is possibly due to differences in tissues of origin and sequencing platforms. The top discriminative features are defined as genes whose gini impurity scores were at least 30 above the mean.

The ML/AI models discussed herein can include any type of ML/AI model or classifier, or combinations thereof, and may be executed on, or applied to, data based on genetic sequencing data of patients to generate values indicative of or corresponding to a health state of the patients (e.g., whether the subject is suffering from CLL or RS, or to inform a decision on treatment). The input to an ML/AI model may be, for example, genetic sequencing data, data on the differences between one or more genes of interest as compared with reference genes corresponding to the genes of interest, or various metrics generated based on such sequencing data or such differences. The output of the ML/AI model may be, for example, a score indicative of a likelihood of the health state. The computing system may report that score itself, an interpretation or derivation of that score, or a recommendation or consequence of the score (e.g., one or more potential treatments, a prognosis, etc.). For example, if the score from the ML/AI model exceeds a threshold value, the computing system may report a positive result (e.g., that a patient is suffering from RS). In addition to other models discussed herein, such as models based on random forest learning techniques, examples of ML/AI models can include neural networks (e.g., a deep neural network (DNN), a convolutional neural network (CNN), a recurrent neural network (RNN) such as a long short-term memory (LSTM) model, combinations thereof, etc.), trained regression models (e.g., linear regression, sparse vector machine (SVM) models, logistic regression, polynomial regression, ridge regression, Lasso regression, Bayesian linear regression, etc.), or other types of classifiers (e.g., naïve Bayes, decision trees, k-nearest neighbors (kNN), extreme gradient boost (XGBoost) models, etc.). The aforementioned machine-learning models may also be utilized for any type of machine-learning or artificial intelligence performed task described herein.

The ML/AI models can be trained using any suitable machine learning technique. For example, ML/AI models can be trained using supervised learning techniques, unsupervised learning techniques, semi-supervised learning techniques, self-supervised learning techniques, or combinations thereof. In example embodiments, the computing system can train the ML/AI models using sets of training data, which may be generated using the techniques described herein. Further details of a process for training the artificial intelligence agent 130 is described in connection with FIG. 3.

Clinical endpoint analysis and statistical analysis. Overall survival (OS) was defined as the interval between date of transformation and death or censored at last follow-up. Survival data were calculated using the method of Kaplan-Meier and curves were compared by log-rank testing using GraphPad.

Data analyses were carried out using GraphPad Prism version 9 and R software version 4. The data are summarized as median or presented as individual values, scatter plots, box and whiskers plot (top of the box is the 75% quantile, and the bottom is the 25%), violin plots, heatmap or with column bar graphs. To compare RS drivers identified with previously reported CLL (n=1063) and DLBCL (n=304) and (n=574) datasets, a 2-sided exact binomial test was performed with Benjamini-Hochberg multiple hypothesis testing correction. To obtain the frequency of RS events in DLBCL cohorts prior to this comparison, we called RS sCNAs in 304 DLBCLs and the 443 primary DLBCLs for which purity was >20%. Event frequencies were compared when an event was detected in both sample sets. RS and CLL drivers co-occurrences were represented by using a Sankey diagram. Significance was evaluated by calculating the probability for acquiring each of the RS drivers considering the acquisition of a given driver in CLL using Fisher's exact test. To evaluate how often a given driver initially occurs during the RS stage in the subset of related RS, the inventors performed the McNemar test. Differences were considered significant when a P value adjusted for multiplicity of testing was <0.05. Overall survival (OS) was defined as the interval between date of transformation and death or censored at last follow-up. Survival data were calculated using the method of Kaplan-Meier and curves were compared by log-rank testing.

Cell Free DNA (cfDNA) Analysis

After sequencing, plasma cfDNA samples were processed and analyzed as reported. To detect RS-specific changes, the inventors undertook the following steps. First, delta copy number changes were analyzed between segments, assigning a positive chromothripsis score when 3 consecutive 1 Mb segments had CN delta ≥0.1, suggested locally fractured genome. Second to assess Richter-specific aneuploidy, the fraction of genome in non-copy-neutral state was evaluated by fraction genome altered (FGA), defining a region as altered if the segment had an event as detected by iCHOR analysis and a CN change ≥0.1 (to filter out low confidence CN changes) and also comparing to a matched CLL sample when available. Third, WGD was assigned to samples where copy-number events had allelic ratios (corrected for iCHOR estimated purites) corresponding to two levels of allele deletions (i.e., 2/0, 1/1 and 2/1 copy-number states) as measured from the main balanced copy-number level (2/2). Lastly, WES was performed on cfDNA (as described herein), which was then examined for RS clonal alterations detected in bulk through phylogenetic reconstruction.

### Consensus Clustering of Genetic Alterations

### Generation of Gene Sample Matrix

All significantly mutated genes (MutSig2CV, q-value≤0.1 and frequency ≥4 cases), significant regions of sCNAs (GISTIC2.0, q-value≤0.1 and frequency >4 cases) were assembled into a gene-by-sample matrix. The entries in the gene-by-sample matrix represent mutations and copy-number (CN) events as follows: non-synonymous mutations, 2; synonymous mutations, 1; no-mutation, 0; high-grade CN gain [CN≥3.4 copies], 2; low-grade CN gain [3.4 copies≥CN ≥2.1 copies], 1; CN neutral, 0; low-grade CN loss [1.1≤CN≤1.9 copies], 1; high-grade CN loss [CN≤1.1 copies], 2; WGD, 5.

### Non-Negative Matrix Factorization Consensus Clustering

To robustly identify clusters of tumors with shared genetic features, the inventors applied a non-negative matrix consensus clustering algorithm with slight modifications. Briefly, the inventors passed the gene-by-sample matrix to the NMF consensus clustering algorithm (testing number of clusters k=2 to 10) and skipped the matrix normalization step so that the distance is calculated directly based on the values in the gene-by-sample matrix. The consensus NMF method runs 20 iterations of NMF starting with different random seeds. The NMF consensus clustering algorithm provided the cluster membership of each sample, the cophenetic coefficient for k=2 to k=10 clusters and silhouette values for the optimal number of clusters, which was k=5. The 7 samples without genetic drivers in the gene-by-sample matrix were assigned to cluster CO. In addition, the inventors identified marker genes differentially expressed across clusters by applying a Fisher's exact test (2×5 table with variant present or absent as one dimension and cluster as the second dimension) and corrected the p-values for multiple hypothesis testing using the BH-FDR procedure. Features with a q-value≤0.1 were selected as cluster features and visualized as a heatmap. Features were annotated with their maximally positive associated cluster, determined by computing the 2×2 Fisher Exact test for all 5 clusters (2×2 table with variant present or absent as one dimension and within-cluster or outside-cluster the second dimension). To ensure robustness given the sample size of 97, 100 subsampling iterations were performed by randomly removing 8 patients in each iteration and calculated a sample-by-sample similarity matrix that reflects the frequency that each of two samples were clustered together in the 100 runs. Finally, UPGMA hierarchical clustering was performed using 1-similarity as a distance metric. To define the final cluster membership, the resulting dendrogram was cut based on the modal number of clusters across the 100 subsampled consensus NMF clustering runs.

### Mutual Exclusivity/Co-Occurrence Estimations.

For each gene of interest, the significance of the co-occurrence or mutual exclusivity for each pair of different events (mutations, amplification, deletion) that affects that gene was calculated using Fisher's exact test, and then false discovery rate was calculated using the Benjamini-Hochberg method.

### Bulk RNA-Sequencing and Data Analyses

High-quality RNA from CLL/RS pairs was extracted, as previously described. Total RNA was quantified using the Quant-iT™ RiboGreen® RNA Assay Kit and normalized to 5 ng/μl. Following plating, 2 μL of ERCC controls (using a 1:1000 dilution) were spiked into each sample. An aliquot of 200 ng for each sample was transferred into library preparation which uses an automated variant of the Illumina TruSeq™ Stranded mRNA Sample Preparation Kit. This method preserves strand orientation of the RNA transcript. It uses oligo dT beads to select mRNA from the total RNA sample, followed by heat fragmentation and cDNA synthesis from the RNA template. The resultant 400 bp cDNA then goes through dual-indexed library preparation: ‘A’ base addition, adapter ligation using P7 adapters, and PCR enrichment using P5 adapters. After enrichment, the libraries were quantified using Quant-iT PicoGreen (1:200 dilution). After normalizing samples to 5 ng/μL, the set was pooled and quantified using the KAPA Library Quantification Kit for Illumina Sequencing Platforms. The entire process was in a 96-well format and all pipetting is done by either Agilent Bravo or Hamilton Starlet. Pooled libraries were normalized to 2 nM and denatured using 0.1 N NaOH prior to sequencing. Flowcell cluster amplification and sequencing were performed according to the manufacturer's protocols using either the HiSeq 2000 or HiSeq 2500 instrument. Each run generated a 101 bp paired-end with an eight-base index barcode read. Data was analyzed using the Broad Picard Pipeline, which includes de-multiplexing and data aggregation.

Data analyses. RNA-seq reads were aligned to the human reference genome hg19 using STAR (v2.4.0.1) 72. Lowly expressed genes with CPM <1 in all samples were filtered out. Differentially expressed (DE) genes were assessed using limma-voom⁷³ in paired mode using sample read counts, with |log₂FC|>1 and adjusted p-value<0.25 as a cutoff. To ensure robustness of the analysis, for the 5 pairs of RS and CLL samples analyzed, DE genes were recalculated iteratively, each time leaving out one sample pair. Genes were rank ordered by their t statistic multiplied by the frequency they were found significant (|log₂FC|>1 and adjusted p-value <0.1) in the leave-one-out analysis. This was used as input for pre-ranked GSEA on HALLMARK pathways (1,000 permutations, weighted enrichment statistics, MSigDB v7.4)⁷⁴.

Rna Clustering of RS Samples and Integration with Genetic Subtypes

RNA-seq data was generated for 39 RS samples. RNA was extracted with Macherey Nagel RNA extraction kit (Macherey-Nagel, Düren, Germany). Total RNA-Seq libraries were generated from 500 ng of total RNA using TruSeq Stranded Total RNA LT Sample Prep Kit with Ribo-Zero Gold (Illumina, San Diego, CA), according to manufacturer's instructions. The final cDNA libraries were checked for quality and quantified using capillary electrophoresis prior to sequencing with HiSeq 4000 sequencing using 1×50 bases protocol.

Two samples were excluded for low tumor purity. Gene counts were pre-processed with ComBat-seq to eliminate possible batch effects and one sample was removed as an outlier. TPMs were computed and genes were filtered out if TPM=0 in at least one sample, median TPM over samples <=0.5, or median TPM over samples >1000. TPMs were then log 2 transformed and top genes by variance (z-score of variance >1) were z-score transformed for downstream analysis. Consensus Clustering using the hierarchical clustering (complete linkage) with spearman distance was used to identify the optimal number of clusters (observed as 5 RNA subtypes), and the resulting consensus matrix was transformed into a distance matrix for hierarchical clustering (complete linkage). The agreement between RNA subtypes and genomically-identified clusters was determined by a Fisher's exact test. Supervised analysis for differentially expressed genes for each genomically-identified cluster was performed using limma-voom as a one-vs-other comparison. Pathway analysis of each genomically-identified cluster was performed using Preranked-GSEA with the MSigDB Hallmark (v7.4) genesets using the LIMMA t-statistic to rank order genes.

Data Deposition. WES, RNA-seq, WGS, and scRNAseq data is deposited in dbgap (Accession number phs002458.v1.p1).

All publications, patents, and patent applications cited in this specification are incorporated herein by reference in their entireties as if each individual publication, patent or patent application were specifically and individually indicated to be incorporated by reference.

What is claimed is:

1. A method of detecting a mutation or genetic alteration in a biological sample, comprising:
obtaining the biological sample from a subject, wherein the subject has been diagnosed with or is suspected of having chronic lymphocytic leukemia (CLL) or wherein the subject has been diagnosed with or is suspected of having Richter's Syndrome (RS);
isolating a nucleic acid sample comprising DNA or RNA from the biological sample; and
detecting
(i) a mutation in at least one of TP53, NOTCH1, IRF2BP2, DNMT3A, SRSF1, EZH2, CCND3, TET2, IRF8, MYC, PIM1, B2M, and PRDM1, or
(ii) at least one genomic alteration selected from the group consisting of: del(1p), del 9(p), amp(8q24.21), HIST1H1E mutation, del(19p13.3), whole genome duplication (WGD), tri(12), SPEN mutation, KRAS mutation, del(17p), del(14q32.11), del(9q), NOTCH1 mutation, IRF2BP2 mutation, TP53 mutation, del(15q15.1), amp(16q23.2), del(2q37.1), SF3B1 mutation, EGR2 mutation, del(13q14.2), IRF8 mutation, PIM1 mutation, amp(7p), del(16q12.1), del(1p35.3), and del(18q22.2).

2. The method of claim 1, wherein the DNA comprises cell-free DNA (cfDNA).

3. The method of claim 2, further comprising isolating the DNA from circulating peripheral blood mononuclear cells (PBMCs) in the biological sample.

4. The method of claim 3, further comprising comparing the mutations in the cfDNA and the DNA from the circulating PBMCs.

5. The method of claim 1, wherein the biological sample is blood, serum, plasma, urine, or saliva.

6. The method of claim 1, wherein detecting the mutation or the at least one gene alteration is performed by nucleic acid sequencing, RT-qPCR, RT-PCR, RNA-seq, Northern blotting, Serial Analysis of Gene Expression (SAGE), or DNA or RNA microarray.

7. The method of claim 1, wherein the mutation is detected in at least one of IRF2BP2, DNMT3A, SRSF1, and EZH2.

8. The method of claim 1, further comprising: detecting a mutation in at least one of SF3B1, GNB1, XPO1, HIST1H1E, HIST1H2AC, EGR2, MGA, CARD11, KRAS, ATM, and BRAF.

9. The method of claim 1, wherein detection of at least one of del(1p), del 9(p), amp (8q24.21), HIST1H1E mutation, del(19p13.3), or whole genome duplication (WGD) indicates RS subtype 1;
wherein detection of at least one of tri(12), SPEN mutation, or KRAS mutation indicates RS subtype 2;
wherein detection of at least one of del(17p), del(14q32.11), del(9q), NOTCH1 mutation, IRF2BP2 mutation, TP53 mutation, del(15q15.1), amp(16q23.2), or del(2q37.1) indicates RS subtype 3;
wherein detection of at least one of SF3B1 mutation, EGR2 mutation, del(13q14.2), or IRF8 mutation indicates RS subtype 4; and
wherein detection of at least one of PIM1 mutation, amp(7p), del(16q12.1), del(1p35.3), or del(18q22.2) indicates RS subtype 5.

10. A method of distinguishing Richter's Syndrome (RS) from chronic lymphocytic leukemia (CLL), comprising:
obtaining a biological sample from a subject diagnosed with CLL;
detecting the presence or absence of a mutation in at least one driver of RS,
wherein the at least one driver of RS is selected from (i) the group consisting of: IRF2BP2, DNMT3A, SRSF1, EZH2, B2M, IRF8, PIM1, HIST1H2AC, PRDM1, CCND3, and TET2, or (ii) the group consisting of amp(9p24), del(16q12), del(18q22), amp(7q21.2), del(1p), amp(11q), amp(1q23), whole genome doubling (WGD), amp(18q21.33), amp(16.23.2), amp(6p22.1), del(9p), del(9q), and amp(7p);
wherein the subject has or will develop RS if a mutation is detected in at least one driver of RS or if at least one genomic alteration is detected, and the subject will not develop RS if a mutation is not detected in at least one driver of RS or if at least one genomic alteration is not detected.

11. The method of claim 10, wherein the biological sample comprises cell-free DNA (cfDNA).

12. The method of claim 10, further comprising isolating the DNA from circulating peripheral blood mononuclear cells (PBMCs) found in the biological sample.

13. The method of claim 12, further comprising comparing the mutations in the cfDNA and the DNA from the circulating PBMCs.

14. The method of claim 10, further comprising:
detecting the presence or absence of at least one genomic alteration selected from the group consisting of del(1p), del 9(p), amp(8q24.21), HIST1H1E mutation, del(19p13.3), whole genome duplication (WGD), tri(12), SPEN mutation, KRAS mutation, del(17p), del(14q32.11), del(9q), NOTCH1 mutation, IRF2BP2 mutation, TP53 mutation, del(15q15.1), amp(16q23.2), del(2q37.1), SF3B1 mutation, EGR2 mutation, del(13q14.2), IRF8 mutation, PIM1 mutation, amp(7p), del(16q12.1), del(1p35.3), and del(18q22.2); and
determining the RS subtype based on the detection of the at least one genomic alteration,
wherein detection of at least one of del(1p), del 9(p), amp(8q24.21), HIST1H1E mutation, del(19p13.3), or whole genome duplication (WGD) indicates RS subtype 1;
wherein detection of at least one of tri(12), SPEN mutation, or KRAS mutation indicates RS subtype 2;
wherein detection of at least one of del(17p), del(14q32.11), del(9q), NOTCH1 mutation, IRF2BP2 mutation, TP53 mutation, del(15q15.1), amp(16q23.2), or del(2q37.1) indicates RS subtype 3;
wherein detection of at least one of SF3B1 mutation, EGR2 mutation, del(13q14.2), or IRF8 mutation indicates RS subtype 4; and
wherein detection of at least one of PIM1 mutation, amp(7p), del(16q12.1), del(1p35.3), or del(18q22.2) indicates RS subtype 5.

15. The method of claim 14, further comprising isolating the DNA from the circulating peripheral blood mononuclear cells (PBMCs) found in the biological sample.

16. The method of claim 15, further comprising comparing the mutations in the cfDNA and the DNA from circulating PBMCs.

17. A computing system comprising:
a processor programmed to perform, or a computer-readable storage device comprising instructions to perform, the following steps:
i) detecting at least one mutation in at least one gene selected from the group consisting of IRF2BP2, DNMT3A, SRSF1, EZH2, B2M, IRF8, PIM1, HIST1H2AC, PRDM1, CCND3, and TET2 and/or at least one genomic alteration selected from the group consisting of amp(9p24), del(16q12), del(18q22), amp(7q21.2), del(1p), amp(11q), amp(1q23), whole genome doubling (WGD), amp(18q21.33), amp(16.23.2), amp(6p22.1), del(9p), del(9q), and amp(7p); in a sequence data from a subject; and
ii) generating a report for medical professional comprising the indication of whether the subject is suffering from chronic lymphocytic leukemia (CLL) or Richter Syndrome (RS) to inform a decision on treatment; or
i) detecting the presence or absence of at least one genomic alteration selected from the group consisting of del(1p), del 9(p), amp(8q24.21), HIST1H1E mutation, del(19p13.3), or whole genome duplication (WGD), tri(12), SPEN mutation, KRAS mutation, del(17p), del(14q32.11), del(9q), NOTCH1 mutation, IRF2BP2 mutation, TP53 mutation, del(15q15.1), amp(16q23.2), del(2q37.1), SF3B1 mutation, EGR2 mutation, del(13q14.2), IRF8 mutation, PIM1 mutation, amp(7p), del(16q12.1), del(1p35.3), and del(18q22.2);
ii) determining the RS subtype based on the at least one genomic alteration,
wherein detection of at least one of del(1p), del 9(p), amp(8q24.21), HIST1H1E mutation, del(19p13.3), or whole genome duplication (WGD) indicates RS subtype 1;
wherein detection of at least one of tri(12), SPEN mutation, or KRAS mutation indicates RS subtype 2;
wherein detection of at least one of del(17p), del(14q32.11), del(9q), NOTCH1 mutation, IRF2BP2 mutation, TP53 mutation, del(15q15.1), amp(16q23.2), or del(2q37.1) indicates RS subtype 3;
wherein detection of at least one of SF3B1 mutation, EGR2 mutation, del(13q14.2), or IRF8 mutation indicates RS subtype 4; and
wherein detection of at least one of PIM1 mutation, amp(7p), del(16q12.1), del(1p35.3), or del(18q22.2) indicates RS subtype 5; and
iii) generating a report for medical professional comprising a prognosis of the subject to inform a treatment decision.

18. The computing system according to claim 17, wherein the computing system is configured to:
receive one or more files comprising sequencing data corresponding to a subject diagnosed with or suspected of having chronic lymphocytic leukemia (CLL);
apply a machine-learning classifier to data based on the sequencing data to obtain an indication of whether the subject is suffering from CLL or RS; and
generate a report for a medical professional based on the indication that informs a decision on treatment.

19. The computing system according to claim 18, wherein the detecting at least one mutation or detecting the presence or absence of at least one genomic alteration comprises:
comparing the sequencing data of the subject with a reference genome sequence.

20. The computing system of claim 17, further comprising:
providing the report, including the indication of whether the subject is suffering from CLL or RS, via a graphical user interface.
